(19) United States
(12) Patent Application Publication
(10) Pub. No.: US 2026/0117070 A1
(43) Pub. Date: Apr. 30, 2026

(54) NEAR-INFRARED FLUORESCENT HEPTAMETHINE DYE CONJUGATES WITH FAVORABLE BLEACHING PROPERTIES

(71) Applicants: HELMHOLTZ ZENTRUM MÜNCHEN DEUTSCHES FORSCHUNGSZENTRUM FÜR GESUNDHEIT UND UMWELT (GMBH), Neuherberg (DE); UNIVERSITÄT ZU KÖLN, Köln (DE)

(72) Inventors: Oliver PLETTENBURG, Hannover (DE); Merle Marie WEITZENBERG, Hannover (DE); Oliver BRUNS, Neuherberg (DE); Thomas BISCHOF, Neuherberg (DE); Bernardo ARUS, Neuherberg (DE); Gina FÜRTJES, Neuherberg (DE); Volker NEUSCHMELTING, Köln (DE)

(21) Appl. No.: 18/868,479

(22) PCT Filed: May 23, 2023
(86) PCT No.: PCT/EP2023/063787

§ 371 Date: Nov. 22, 2024

(30) Foreign Application Priority Data
May 23, 2022 (EP) 22174958.3

(51) Int. Cl. C09B 23/10; A61K 49/00

(52) U.S. Cl. CPC C09B 23/107; A61K 49/0032; A61K 49/0056

(57) ABSTRACT

The present invention relates to near-infrared fluorescent heptamethine dyes and conjugates of heptamethine dyes with favourable biodistribution and bleaching properties.

### TECHNICAL FIELD OF THE INVENTION

The present invention relates to near-infrared fluorescent heptamethine dyes and conjugates of heptamethine dyes with favourable biodistribution and bleaching properties.

### BACKGROUND ART

Target-specific contrast agents enable the non-invasive visualization of molecular structures and processes in cells, tissues or living organisms and therefore are valuable tools for biomolecular imaging. They compose of a targeted ligand that is bound to the label via a linker. In the case of optical imaging, the label is a fluorescent dye with a) a conjugation handle and b) peak emission >780 nm, which allows imaging in the Near and Shortwave Infrared. In particular, the latter allows comparatively deep penetration into tissue.

Applications for fluorescent target-specific contrast agents range from medical diagnostics and surgical navigation to basic biomedical research and all require a favourable target to background ratio. In vivo, this results from the combination of specific binding of the probe to its target structure and efficient clearance of free or unspecifically bound probe. Many applications furthermore require imaging over a prolonged period. Continuous irradiation is accompanied by photobleaching, the gradual decrease in emitted fluorescence due to photochemical alteration of the fluorophore.

Indocyanine green (ICG, NIR dye, gold standard) and 5-aminolevulinic acid (5-ALA, converted to protoporphyrin IX, VIS dye) are routinely used in the clinic for optical imaging. Neither of them has a conjugation handle and they thus cannot be used to label specific target tissues. Besides this, photostability poses a significant problem for in vivo experiments. For ICG this is the case when it is not used a's a contrast agent for purely intravascular FI angiography, but plasma protein-bound a's a contrast agent via EPR for tumour imaging (1). The lack of photostability of 5-ALA (PPIX) is a well-known problem in clinical research (2).

The majority of targeted fluorescent contrast agents currently propelling clinical trials consist of IRDye®8000W.

S̆tacková et al. disclosed an approach to a substituted heptamethine cyanine chain by the ring opening of Zincke Salts (3). However, in contrast to the present invention, the compounds of S̆tackova et al. do not disclose compounds which exhibit additional sulfonate groups on both aromatic rings and an electron withdrawing amide group attached to a polyene scaffold in combination which relate to different improved properties of the inventive compounds, a's discussed below.

IRDye®8000W is a commercially available heptamethine cyanine dye that comes with a conjugation handle. Its conjugates are currently propelling more than 20 Phase I and Phase II clinical trials (4), (5). The compounds of the present invention and IRDye®8000W are of similar molecular weight and have the same degree of sulfonation/net charge which results in similar in vivo behaviour. In difference to the compounds of the present invention, IRDye®8000W has an electron donating phenol attached to the polyene scaffold that increases the nucleophilicity of the conjugated pi-system and makes it more prone to singlet oxygen driven photobleaching. IRDye®8000W is also characterized by long unspecific tissue half-live, limiting imaging applications shortly after injection.

There is a variety of conjugateable NIR-fluorescent dyes known in the prior art of which publications (6)-(14) represent a selection. However, there is no conjugateable NIR-fluorescent dye with an electron withdrawing amide group attached to a polyene scaffold in combination with a high degree of sulfonic acid substituents at the indocyanine backbone a's described in the present invention.

Thus, there is a need for further fluorescent dyes.

### SUMMARY OF THE INVENTION

A compound according to formula (I),

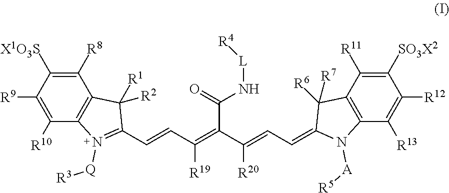

wherein
R¹, R², R⁶, R⁷, R¹⁴, R¹⁵, R¹⁶, R¹⁷, and R¹⁸ are independently selected from the group consisting of H and (C₁-C₆)alkyl, preferably methyl;
R³ is —SO₃X³, or COOH;
R⁴ is selected from the group consisting of -ethinyl, -azide,

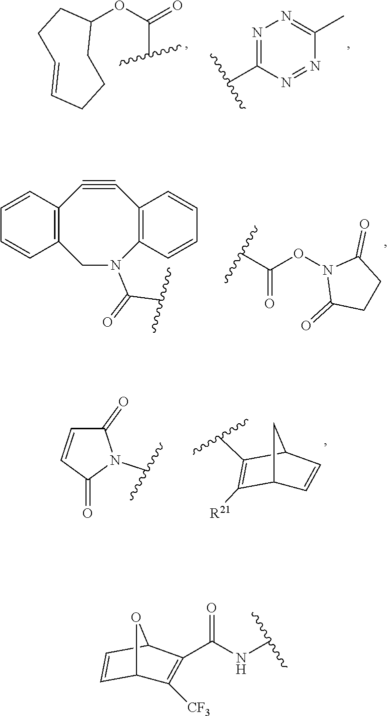

—COOH, NH₂, Cl, Br, I, —OSO₂CF₃, —COSO₂CH₃, —OH, vinyl, —SH;
R⁵ is —SO₃X⁴;
R⁸, R⁹, R¹⁰, R¹¹, R¹² and R¹³ are independently selected from the group consisting of H, CN, —COO(C₁-C₆)alkyl, —CONH(C₁-C₆)alkyl, Cl, Br, I, F, —CF₃, —O(C₁-C₆), —NR¹⁴R¹⁵, —NHC(O)(C₁-C₆)alkyl, —NHC(O)NH(C₁-C₆)alkyl), —SO₃H, —SO₂NH₂, -(C₁-C₆)alkyl, preferably H;
R¹⁹, R²⁰ are independently selected from the group consisting of H, -(C₁-C₆)alkyl, —O(C₁-C₆)alkyl, Cl, F, Br, CN; C(O)NH₂, preferably H;
R²¹ is H, -(C₁-C₁₀)alkyl;
L is a group consisting of one or more and/or one or more of each of the elements selected from the group: —(CH₂)ₙ₁—, -(C₁-C₆)cycloalkylene-, -(C₁-C₆)heterocycloalkylene-, —O—, —NR¹⁶—, —NH—, —NHC(O)—, —C(O)NR¹⁷—, —CH(OH)—, and —CH(OR¹⁸)—;
n¹ is an integer between 1 to 30, preferably 1 to 20, more preferably 1 to 10;
X¹, X², X³, and X⁴ are independently selected from a cation, a proton or absent, preferably the cation is a monovatent cation or a divalent cation, more preferably the cation is a monovalent cation most preferably an alkali metal cation or an ammonium cation, particularly preferred the cation is independently selected from the group consisting of Na⁺, K⁺; and NH₄⁺.
Q is —(CH₂)ₙ₂—,
wherein optionally one or more of each H in —(CH₂)ₙ₂— may be replaced by -(C₁-C₁₀)alkyl;
n² is an integer between 1 to 10, preferably 1 to 6, more preferably 1 to 4;
A is —(CH₂)ₙ₃—,
wherein optionally one or more of each H in —(CH₂)ₙ₃— may be replaced by -(C₁-C₁₀)alkyl;
n³ is an integer between 1 to 10, preferably 1 to 6, more preferably 1 to 4.

The invention is further directed to a compound according to formula (II)

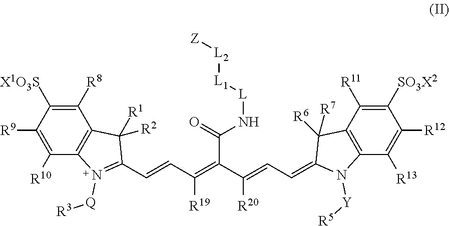

wherein
R¹, R², R⁶, R⁷, R¹⁴, R¹⁵, R¹⁶, R¹⁷, and R¹⁸ are independently selected from the group consisting of
H and (C₁-C₆)alkyl, preferably methyl;
R³ is —SO₃X³, or COOH;
Z is a protein, mono- or polyvalent saccharide, peptide, small molecule, antibody, oligonucleotide, nanobody, polymer;
R⁵ is —SO₃X⁴;
R⁸, R⁹, R¹⁰, R¹¹, R¹² and R¹³ are independently selected from the group consisting of H, CN, —COO(C₁-C₆)alkyl, —CONH(C₁-C₆)alkyl, Cl, Br, I, F, —CF₃, —O(C₁-C₆), —NR¹⁴R¹⁵, —NHC(O)(C₁-C₆)alkyl, —NHC(O)NH(C₁-C₆)alkyl), -(C₁-C₆)alkyl, —SO₃H, —SO₂NH₂, preferably H;
R¹⁹, R²⁰ are independently selected from the group consisting of H, -(C₁-C₆)alkyl, —O(C₁-C₆)alkyl, Cl, F, Br, CN; C(O)NH₂, preferably H;
R²¹ is H, -(C₁-C₁₀)alkyl;
X¹, X², X³, and X⁴ are independently selected from a cation, a proton or absent, preferably the cation is a monovalent cation or a divalent cation, more preferably the cation is a monovalent cation, most preferably an alkali metal cation or an ammonium cation, particularly preferred the cation is independently selected from the group consisting of Na⁺, K⁺; and NH₄⁺;
Q is —(CH₂)ₙ₂—,
wherein optionally one or more of each H in —(CH₂)ₙ₂— may be replaced by -(C₁-C₁₀)alkyl;
n² is an integer between 1 to 10, preferably 1 to 6, more preferably 1 to 4;
Y is —(CH₂)ₙ₃—,
wherein optionally one or more of each H in —(CH₂)ₙ₂— may be replaced by -(C₁-C₁₀)alkyl;
n³ is an integer between 1 to 10, preferably 1 to 6, more preferably 1 to 4;
L is a group consisting of one or more and/or one or more of each of the elements selected from the group: —(CH₂)ₙ₁—, -(C₁-C₆)cycloalkylene-, -(C₁-C₆)heterocycloalkylene-, —O—, —NR¹⁶—, —NH—, —NHC(O)—, —C(O)NR¹⁷—, —CH(OH)—, and —CH(OR¹⁸)—;
n¹ is an integer between 1 to 30, preferably 1 to 20, more preferably 1 to 10;
L₁ is a suitable linking moiety; preferably L₁ is selected from the group consisting of

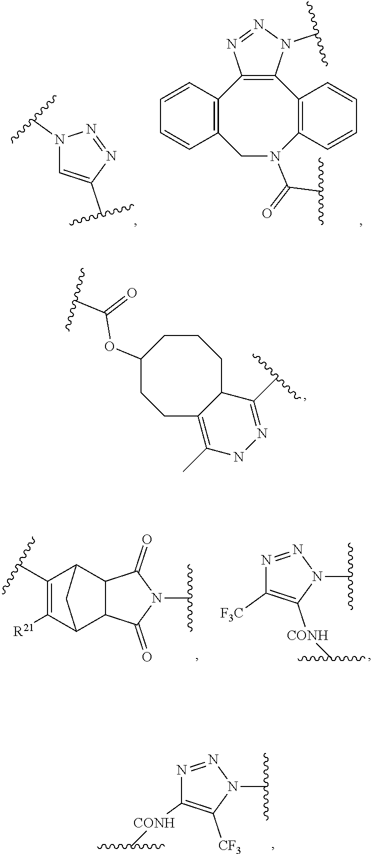

### —CO—;

L₂ is is a group consisting of one or more and/or one or more of each of the elements selected from the group: —(CH₂)ₙ₁—, -(C₁-C₆)cycloalkylene-, -(C₁-C₆)heterocycloalkylene-, —O—, —NR¹⁶—, —NH—, —NHC(O)—, —C(O)NR¹⁷—, —CH(OH)—, and —CH(OR¹⁸)—;
The invention is further directed to the use of the compounds a's fluorescent dye.

The invention is further directed to the use of the compounds in diagnosis, or for intraoperative surgery, in particular for use a's contrast agent.

In a further aspect, the indication to be diagnosed or treated by intraoperative surgery is selected from the group consisting of inflammatory diseases, infectious diseases and cancer.

In a further aspect of the invention, the inflammatory disease is selected from the group consisting of otitis media, cholesteatoma, allergic rhinitis and chronic rhinosinusitis, chronic inflammation of tonsils, adenoids, and laryngitis.

In another aspect of the invention, the infectious disease is selected from the group consisting of implant infection (e.g. hip, dental), secondary infections of the inner ear, e.g. accompanying cholesteatoma. This comprises infections caused by gram positive or gram negative bacteria, responding to antibiotic treatment, resistant or multi-drug resistant, in particular by ESKAPE strains Enterococcus faecium, Staphylococcus aureus, Klebsiella pneumoniae, Acinetobacter baumannii, Pseudomonas aeruginosa, and Enterobacter spp.

In a further aspect, diagnosing of cancer or treating cancers by intraoperative surgery relates to visualizing the primary tumor and/or metastatic lesions in situ during surgery selected from the group meningioma, glioblastoma, NSCLC, pancreatic cancer, neuroendocrine tumors, lung cancer, breast cancer, meningiomas.

Another aspect is the visualization of risk structures during surgery to isolate or protect them. Such risk structures include nerves, blood vessels, lymphatic vessels, ureter, adrenals, pancreatic islets, pituitary gland, thyroid gland, parathyroid gland among others.

The compounds of the present invention, if used a's fluorescent dyes and contrast agents, show a fast clearance and therefore favourable target to background ratios compared to reference conjugates using the known dye IRDye800. Moreover, the compounds of the present invention also exhibit a higher photostability which allow their improved application in fluorescence optical imaging, especially those that require laser exposure, over a prolonged period. The higher photostability is in particular beneficial for application of the inventive compounds in fluorescence-guided surgery. It has particularly been found that surprisingly the combination of a sulfonated aromatic system and an electron withdrawing amide attached to the polyene backbone is required for providing superior bleaching properties (see FIG. 3).

### BRIEF DESCRIPTION OF THE FIGURES

FIG. 1: Tissue and blood clearance of TOC-sNIR (sstr2click1) and TOC-IRDye®8000W (sstr2click2). For the data acquisition, mice were imaged during i.v. injection (5 nmol in 100 uL PBS). To avoid bleaching effects, the laser was triggered and only one image was acquired every 20 sec. power density was 50 mW/cm².

FIG. 2: Bleaching kinetics of TOC-sNIR (sstr2click1), TATE-sNIR (sstr2click4) and TOC-IRDye®8000W (sstr2click2) in vivo. For the data acquisition, mice were i.v. injected with 5 nmol dye conjugate in 100 μL PBS. The bleaching experiment was performed 3 h after injection. Two 785 nm lasers were used, differing in intensity and the size of the illuminated area. For local bleaching of the kidney, the kidney was irradiated with a power density of 200 mW/cm² over a period of 30 min. The whole-body images were taken with a power density of 50 mW/cm² before and after bleaching. After completion of the experiment, we noticed that TOC-sNIR (green curve), had not yet reached its end point (slope=0) like TOC-IRDye (orange curve). For this reason, we performed another bleaching experiment with TATE-sNIR, for which we used a local power density of 450 mW/cm². To compare the data with those from TOC-IRDye, we multiplied the time axis of TATE-sNIR by a factor of (450/200). Since both TOC-IRDye and TATE-sNIR reached their endpoints, the data were normalized to maximum and minimum. The peptides TOC and TATE differ only with respect to the oxidation state of the C-terminus.

FIG. 3: Bleaching studies in vitro have been performed.

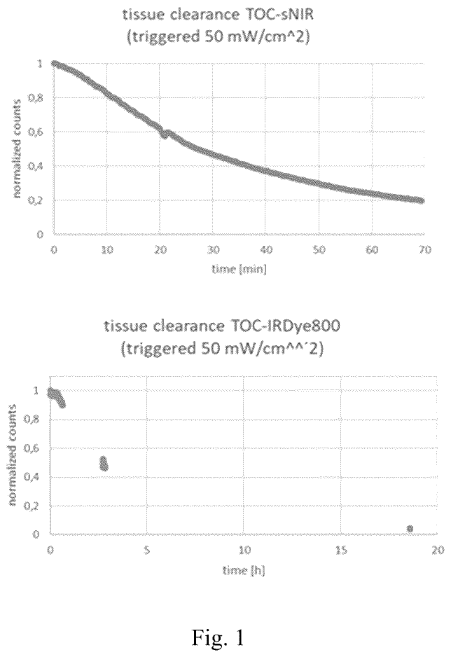

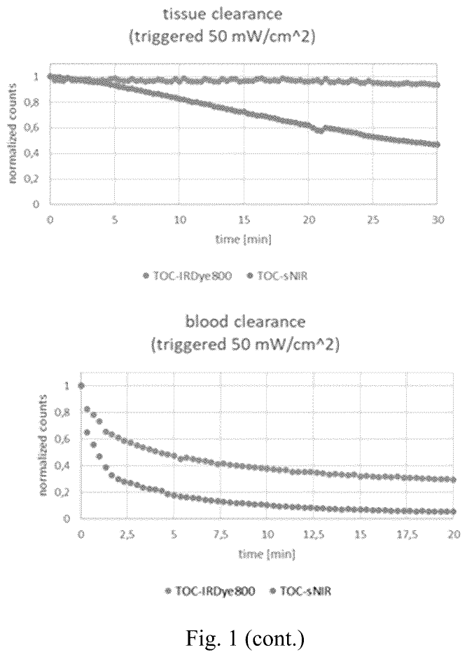

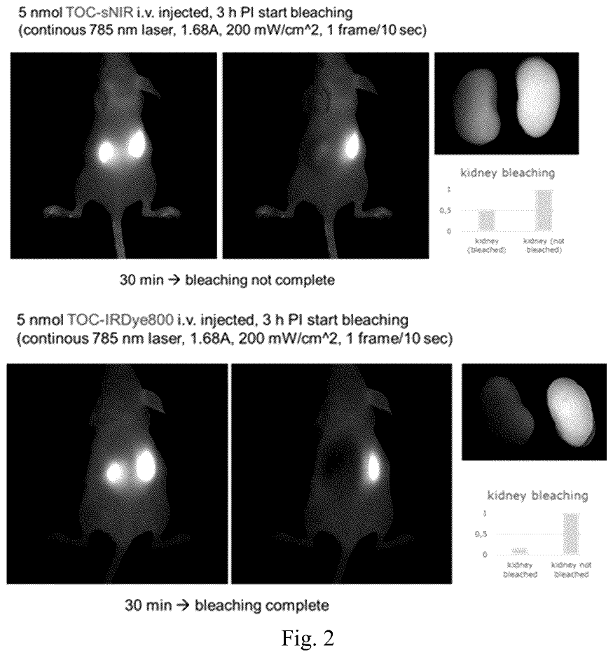

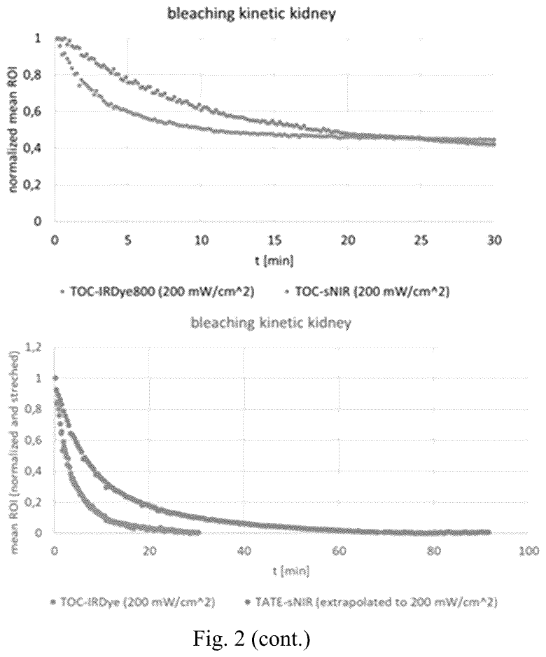

3A: Structural formulas of the alkyne-functionalized versions of NIR (dyealkin2) and sNIR (dyealkin5) a's well a's IRDye®8000W carboxylate a's they were used for the bleaching experiments. Bleaching kinetics of NIR, sNIR and IRDye®8000W are shown in 3B) water and 3C) FBS. Under continuous 785 nm irradiation, sNIR is bleaching 3.5-times slower than NIR, ICG and IRDye®8000W in water. In fetal bovine serum (FBS), sNIR is 2.5-times more photostable than NIR, IRDye®8000W and 1.6-times more stable than ICG. Overall, sNIR has favourable photobleaching properties in comparison to its less sulfonated analogue NIR, Indocyanine Green (ICG, clinically approved dye without conjugation handle) and IRDye®8000W. These results prove that surprisingly the combination of the sulfonated aromatic system and the electron withdrawing amide attached to the polyene backbone is required for providing superior bleaching properties.

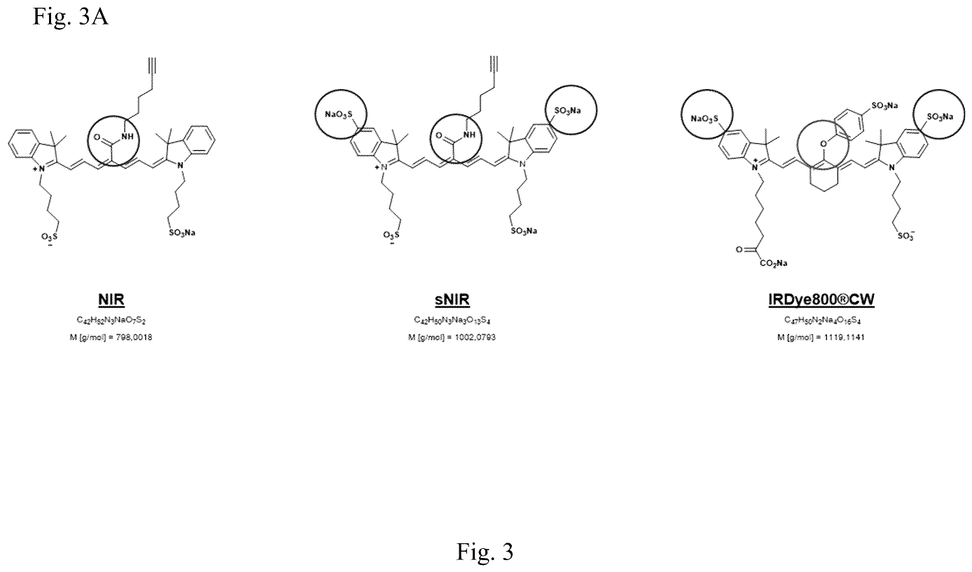

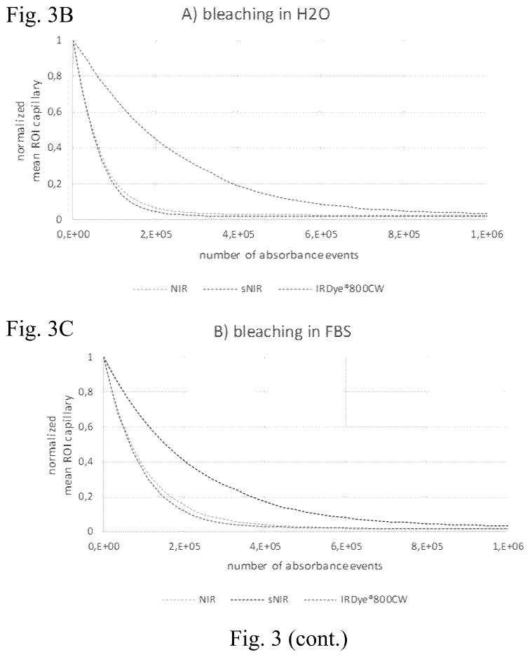

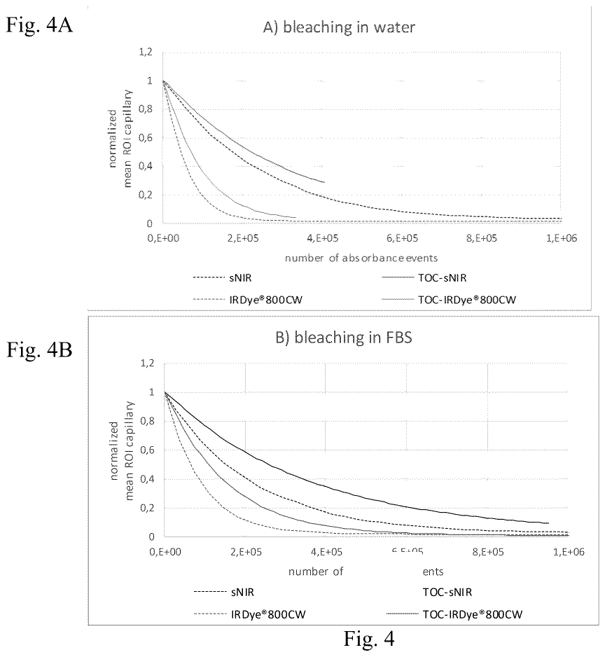

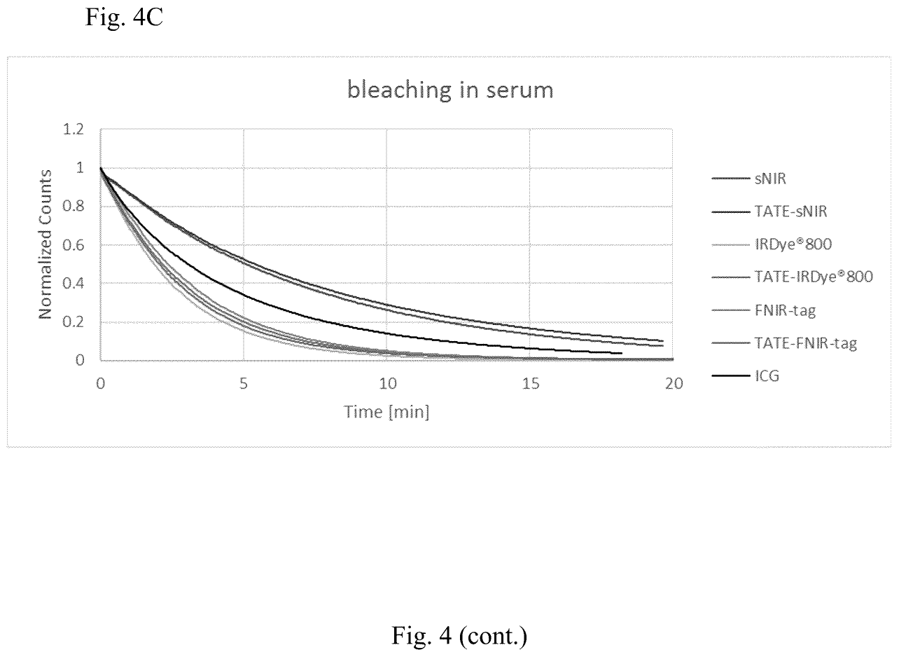

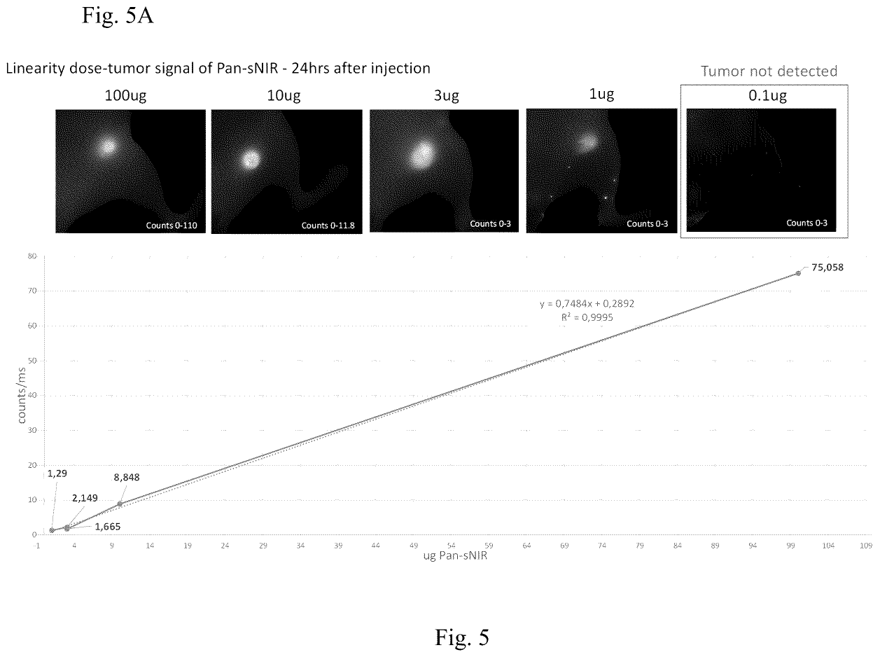

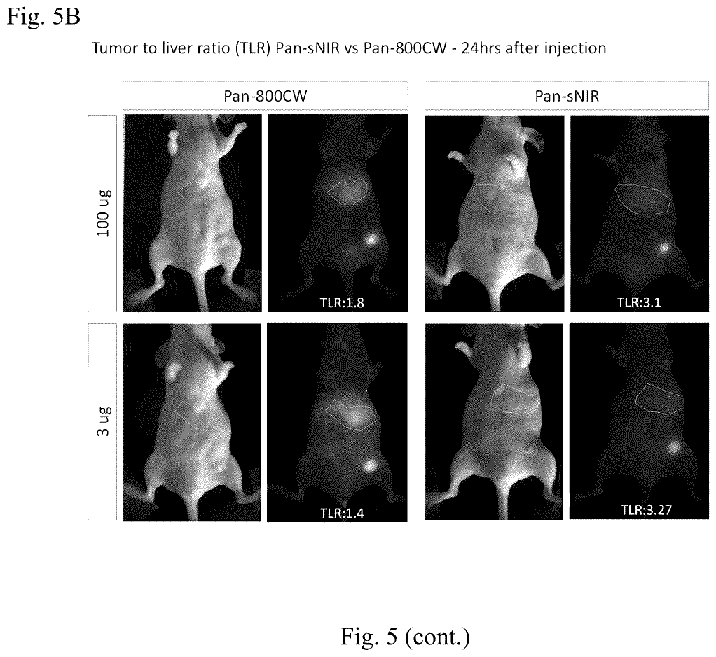

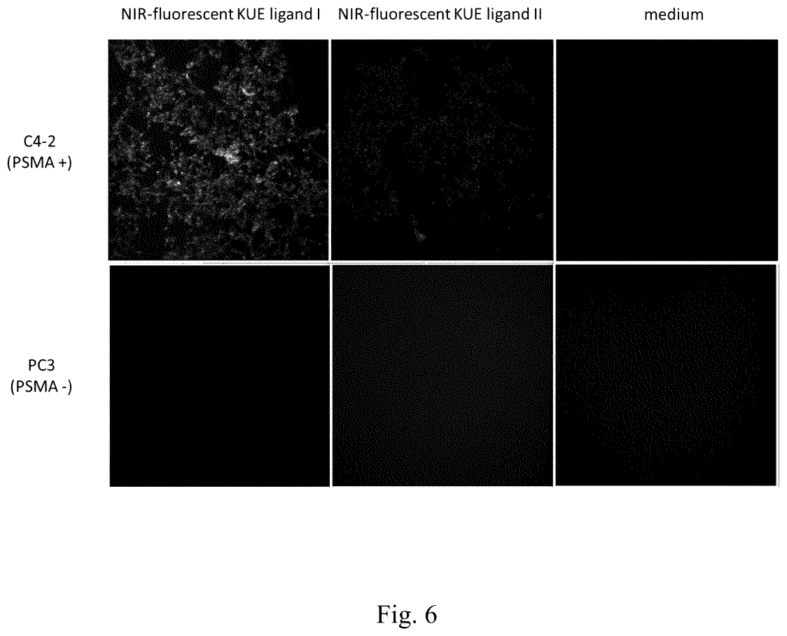

FIG. 4: Bleaching kinetics of sNIR, TOC-sNIR, IRDye®8000W and TOC-IRDye®8000W in A) water and B) FBS. Conjugation of sNIR and IRDye®8000W to the TOC-peptide improved photostability of the probe, but did not change the general trend of photobleaching described above for FIG. 3. 4C): Bleaching in vitro: Capillaries filled with 2.5 μM solutions in serum were continuously irradiated using a 785 nm laser (100 mW/cm\hat{ }2) and frames were acquired for 20 minutes. Bleaching kinetics were extracted by analyzing the mean counts in an ROI overtime

FIG. 5: Comparing Pan-sNIR (NIR-fluorescent Panitumumab) and Pan-IRDye800CW in tumor bearing mice post intraveneous injection. Mice were generated by subcutaneous implantation of MDA-MB-468 cells. A) In vivo distribution of monoclonal antibody Panitumumab conjugated to sNIR dye showed linear correlation between tumor signal and injected dose (100 μg−1 μg). 0.1 μg dose could not be detected at the tumor. B) Mice injected with 3 μg (2 animals/group) or 100 μg of Pan-sNIR (1 animal/group) showed ˜3.1-3.2× tumor signal than the liver 24 hrs after injection. Instead, same doses of Pan-8000W showed ˜1.4-1.8× tumor signal over the liver.

FIG. 6: Cell uptake assay using NIR-fluorescent KUE-ligands I and II on PSMA-positive C4-2 and PSMA-negative PC3 cells.

### DETAILED DESCRIPTION OF THE INVENTION

The solution of the present invention is described in the following, exemplified in the appended examples, illustrated in the Figures and reflected in the claims.

### Definitions

It is noted that a's used herein, the singular forms Z“a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

The term “and/or” wherever used herein includes the meaning of “and”, “or” and “all or any other combination of the elements connected by said term”.

For example, less than 20 means less than the number indicated. Similarly, more than or greater than means more than or greater than the indicated number, f.e. more than 80% means more than or greater than the indicated number of 80%.

Throughout this specification and the claims which follow, unless the context requires otherwise, the word “comprise”, and variations such a's “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

The term “alkyl” refers in context of the present invention to a monoradical of a saturated straight or branched hydrocarbon. Preferably, the alkyl group comprises from 1 to 10 carbon atoms, i.e., 1, 2, 3, 4, 5, 6, 7, 8, 9, or 10, carbon atoms, more preferably 1 to 8 carbon atoms, such a's 1 to 6 or 1 to 4 carbon atoms. Exemplary alkyl groups include methyl, ethyl, propyl, iso-propyl, butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, n-heptyl, iso-heptyl, n-octyl, 2-ethyl-hexyl, n-nonyl, n-decyl, and the like.

The term “cycloalkyl” or “cycloaliphatic” represents cyclic non-aromatic versions of “alkyl” and “alkenyl” with preferably 3 to 14 carbon atoms, such a's 3 to 10 carbon atoms, i.e., 3, 4, 5, 6, 7, 8, 9, or 10 carbon atoms, more preferably 3 to 6 carbon atoms, even more preferably 6 carbon atoms. Exemplary cycloalkyl groups include cyclopropyl, cyclopropenyl, cyclobutyl, cyclobutenyl, cyclopentyl, cyclopentenyl, cyclohexyl, cyclohexenyl, cycloheptyl, cycloheptenyl, cyclooctyl, cyclooctenyl, cyclononyl, cyclononenyl, cylcodecyl, and adamantyl. The term “cycloalkyl” is also meant to include bicyclic and tricyclic versions thereof. If bicyclic rings are formed it is preferred that the respective rings are connected to each other at two adjacent carbon atoms, however, alternatively the two rings are connected via the same carbon atom, i.e., they form a spiro ring system or they form “bridged” ring systems. Preferred examples of cycloalkyl include C₃-C₈-cycloalkyl, in particular cyclopropyl, cyclobutyl, cyclopentyl, cyclohexyl, cycloheptyl, cyclooctyl, spiro[3,3]heptyl, spiro[3,4]octyl, spiro[4,3]octyl, bicyclo[4.1.0]heptyl, bicyclo[3.2.0]heptyl, bicyclo[2.2.1]heptyl, bicyclo[2.2.2]octyl, bicyclo[5.1.0]octyl, and bicyclo[4.2.0]octyl.

The term “cycloalkylene” means a cycloalkyl group a's defined above in which one hydrogen atom has been removed resulting in a diradical. The cycloalkylene may link two atoms or moieties via the same carbon atom (1,1-cycloalkylene, i.e., a geminal diradical) or via two carbon atoms (1,2-cycloalkylene).

The term “heterocyclyl” means a cycloalkyl group a's defined above in which from 1, 2, 3, or 4 carbon atoms in the cycloalkyl group are replaced by heteroatoms of O, S, or N. Preferably, in each ring of the heterocyclyl group the maximum number of O atoms is 1, the maximum number of S atoms is 1, and the maximum total number of O and S atoms is 2. The term “heterocyclyl” is also meant to encompass partially or completely hydrogenated forms (such a's dihydro, tetrahydro or perhydro forms) of the above-mentioned heteroaryl groups. Exemplary heterocyclyl groups include morpholino, isochromanyl, chromanyl, pyrrolidinyl, imidazolidinyl, pyrazolidinyl, piperidinyl, piperazinyl, indolinyl, isoindolinyl, di- and tetrahydrofuranyl, di- and tetrahydrothienyl, di- and tetrahydrooxazolyl, di- and tetrahydroisoxazolyl, di- and tetrahydrooxadiazolyl (1,2,5- and 1,2,3-), dihydropyrrolyl, dihydroimidazolyl, dihydropyrazolyl, di- and tetrahydrotriazolyl (1,2,3- and 1,2,4-), di- and tetrahydrothiazolyl, di- and tetrahydrothiazolyl, di- and tetrahydrothiadiazolyl (1,2,3- and 1,2,5-), di- and tetrahydropyridyl, di- and tetrahydropyrimidinyl, di- and tetrahydropyrazinyl, di- and tetrahydrotriazinyl (1,2,3-, 1,2,4-, and 1,3,5-), di- and tetrahydrobenzofuranyl (1- and 2-), di- and tetrahydroindolyl, di- and tetrahydroisoindolyl, di- and tetrahydrobenzothienyl (1- and 2), di- and tetrahydro-1H-indazolyl, di- and tetrahydrobenzimidazolyl, di- and tetrahydrobenzoxazolyl, di- and tetrahydroindoxazinyl, di- and tetrahydrobenzisoxazolyl, di- and tetrahydrobenzothiazolyl, di- and tetrahydrobenzisothiazolyl, di- and tetrahydrobenzotriazolyl, di- and tetrahydroquinolinyl, di- and tetrahydroisoquinolinyl, di- and tetrahydrobenzodiazinyl, di- and tetrahydroquinoxalinyl, di- and tetrahydroquinazolinyl, di- and tetrahydrobenzotriazinyl (1,2,3- and 1,2,4-), di- and tetrahydropyridazinyl, di- and tetrahydrophenoxazinyl, di- and tetrahydrothiazolopyridinyl (such a's 4,5,6-7-tetrahydro[1,3]thiazolo[5,4-c]pyridinyl or 4,5,6-7-tetrahydro[1,3]thiazolo[4,5-c]pyridinyl, e.g., 4,5,6-7-tetrahydro[1,3]thiazolo[5,4-c]pyridin-2-yl or 4,5,6-7-tetrahydro[1,3]thiazolo[4,5-c]pyridin-2-yl), di- and tetrahydropyrrolothiazolyl (such a's 5,6-dihydro-4H-pyrrolo[3,4-d][1,3]thiazolyl), di- and tetrahydrophenothiazinyl, di- and tetrahydroisobenzofuranyl, di- and tetrahydrochromenyl, di- and tetrahydroxanthenyl, di- and tetrahydrophenoxathiinyl, di- and tetrahydropyrrolizinyl, di- and tetrahydroindolizinyl, di- and tetrahydroindazolyl, di- and tetrahydropurinyl, di- and tetrahydroquinolizinyl, di- and tetrahydrophthalazinyl, di- and tetrahydronaphthyridinyl (1,5-, 1,6-, 1,7-, 1,8-, and 2,6-), di- and tetrahydrocinnolinyl, di- and tetrahydropteridinyl, di- and tetrahydrocarbazolyl, di- and tetrahydrophenanthridinyl, di- and tetrahydroacridinyl, di- and tetrahydroperimidinyl, di- and tetrahydrophenanthrolinyl (1,7-, 1,8-, 1,10-, 3,8-, and 4,7-), di- and tetrahydrophenazinyl, di- and tetrahydrooxazolopyridinyl, di- and tetrahydroisoxazolopyridinyl, di- and tetrahydropyrrolooxazolyl, and di- and tetrahydropyrrolopyrrolyl. Exemplary 5- or 6-memered heterocyclyl groups include morpholino, pyrrolidinyl, imidazolidinyl, pyrazolidinyl, piperidinyl, piperazinyl, di- and tetrahydrofuranyl, di- and tetrahydrothienyl, di- and tetrahydrooxazolyl, di- and tetrahydroisoxazolyl, di- and tetrahydrooxadiazolyl (1,2,5- and 1,2,3-), dihydropyrrolyl, dihydroimidazolyl, dihydropyrazolyl, di- and tetrahydrotriazolyl (1,2,3- and 1,2,4-), di- and tetrahydrothiazolyl, di- and tetrahydroisothiazolyl, di- and tetrahydrothiadiazolyl (1,2,3- and 1,2,5-), di- and tetrahydropyridyl, di- and tetrahydropyrimidinyl, di- and tetrahydropyrazinyl, di- and tetrahydrotriazinyl (1,2,3-, 1,2,4-, and 1,3,5-), and di- and tetrahydropyridazinyl.

The term “heterocycloalkylene” a's used herein means a heterocyclyl group a's defined above and in which one hydrogen atom has been removed from a nitrogen atom, the same carbon atom or different carbon atoms, resulting in a diradical. The heterocycloalkylene may link two atoms or moieties via the same carbon atom or via two carbon atoms.

### Compounds

The invention is directed to a compound according to formula (I),

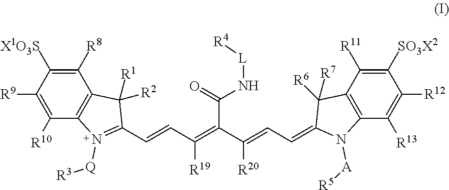

wherein

R¹, R², R⁶, R⁷, R¹⁴, R¹⁵, R¹⁶, R¹⁷, and R¹⁸ are independently selected from the group consisting of H and (C₁-C₆)alkyl, preferably methyl.

R³ is —SO₃X³, or COOH.

R⁴ is selected from the group consisting of -ethinyl, -azide,

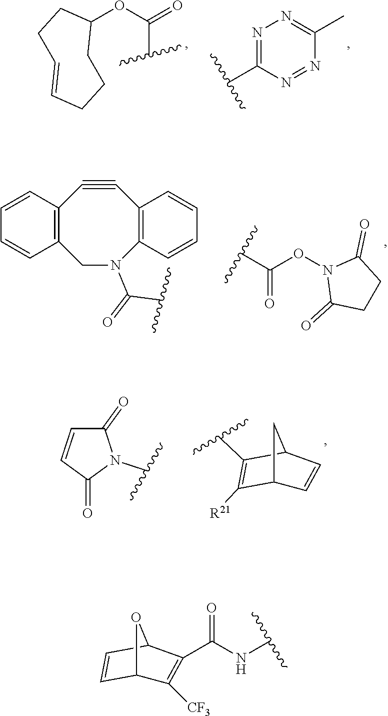

—COOH, NH₂, Cl, Br, I, —OSO₂CF₃, —COSO₂CH₃, —OH, vinyl, —SH.

R⁵ is —SO₃X⁴.

R⁸, R⁹, R¹⁰, R¹¹, R¹² and R¹³ are independently selected from the group consisting of H, CN, —COO(C₁-C₆)alkyl, —CONH(C₁-C₆)alkyl, Cl, Br, I, F, —CF₃, —O(C₁-C₆), —NR¹⁴R¹⁵, —NHC(O)(C₁-C₆)alkyl, —NHC(O)NH(C₁-C₆)alkyl), —SO₃H, —SO₂NH₂, -(C₁-C₆)alkyl, preferably H.

R¹⁹, R²⁰ are independently selected from the group consisting of H, -(C₁-C₆)alkyl, —O(C₁-C₆)alkyl, Cl, F, Br, CN; C(O)NH₂, preferably H.

R²¹ is H, -(C₁-C₁₀)alkyl.

L is a group consisting of one or more and/or one or more of each of the elements selected from the group: —(CH₂)ₙ₁—, -(C₁-C₆)cycloalkylene-, -(C₁-C₆)heterocycloalkylene-, —O—, —NR¹⁶—, —NH—, —NHC(O)—, —C(O)NR¹⁷—, —CH(OH)—, and —CH(OR¹⁸)—.

Preferably L is selected from the group consisting of —CH₂(CH₂OCH₂)ₙ₁CH₂—, —(CH₂)ₙ₂—, —CH₂(CH₂NHCH₂)ₙ₁CH₂—, —CH₂(CH₂NR¹⁶CH₂)ₙ₁CH₂—, —CH₂(CH₂OCH₂)ₙ₁CH₂C(O)(CH₂)ₙ₄—, —CH₂(CH₂NHCH₂)ₙ₁CH₂C(O)(CH₂)ₙ₄—, —CH₂(CH₂NR¹⁶CH₂)ₙ₁CH₂C(O)(CH₂)ₙ₄—.

n¹ is an integer between 1 to 30, preferably 1 to 20, more preferably 1 to 10.

n² is an integer between 1 to 10, preferably 1 to 6, more preferably 1 to 4.

n⁴ is an integer between 1 to 10, preferably 1 to 6, more preferably 1 to 4.

X¹, X², X³, and X⁴ are independently selected from a cation, a proton or absent, preferably the cation is a monovalent cation or a divalent cation, more preferably the cation is a monovalent cation most preferably an alkali metal cation or an ammonium cation, particularly preferred the cation is independently selected from the group consisting of Na⁺, K⁺; and NH₄⁺;

If X¹, X², X³, and/or X⁴ are absent the underlying sulfonyl group is present a's anion.

Q is —(CH₂)ₙ₂—,
wherein optionally one or more of each H in —(CH₂)ₙ₂— may be replaced by -(C₁-C₁₀)alkyl.

A is —(CH₂)ₙ₃—,
wherein optionally one or more of each H in —(CH₂)ₙ₃— may be replaced by -(C₁-C₁₀)alkyl.

n³ is an integer between 1 to 10, preferably 1 to 6, more preferably 1 to 4.

In a further embodiment, in formula (I),

R¹, R², R⁶, R⁷ are Methyl.

R³ is —SO₃X³.

R⁴ is selected from the group consisting of -ethinyl, -azide,

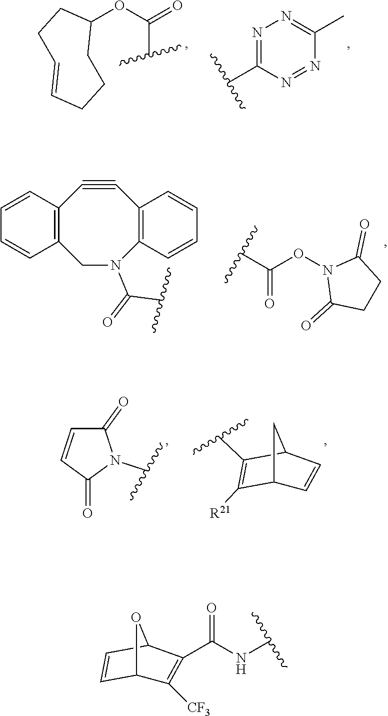

—COOH, and NH₂, preferably ethinyl, -azide,

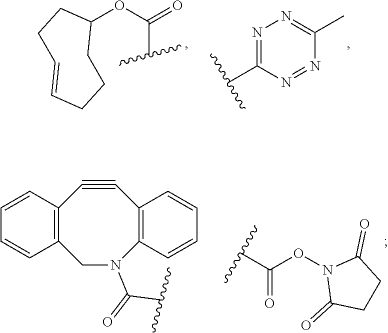

R⁵ is —SO₃X⁴.

R⁸, R⁹, R¹⁰, R¹¹, R¹² and R¹³ are H.

R¹⁶, R¹⁷, and R¹⁸ are independently selected from the group consisting of H and (C₁-C₆)alkyl, preferably methyl.

R¹⁹ and R²⁰ are H.

R²¹ is H, -(C₁-C₁₀)alkyl.

L is a group consisting of one or more and/or one or more of each of the elements selected from the group: —(CH₂)ₙ₁—, —O—, —NR¹⁶—, —NHC(O)—, —C(O)NR¹⁷—, —CH(OH)—, and —CH(OR¹⁸)—; preferably —(CH₂)ₙ₁—, and —O—.

n¹ is an integer between 1 to 30, preferably 1 to 20, more preferably 1 to 10.

X¹, X², X³, and X⁴ are independently selected from a cation, a proton or absent, preferably the cation is a monovatent cation or a divalent cation, more preferably the cation is a monovalent cation most preferably an alkali metal cation or an ammonium cation, particularly preferred the cation is independently selected from the group consisting of Na⁺, K⁺; and NH₄⁺.

Q is —(CH₂)ₙ₂—.
wherein optionally one or more of each H in —(CH₂)ₙ₂— may be replaced by -(C₁-C₁₀)alkyl.

n² is an integer between 1 to 10, preferably 1 to 6, more preferably 1 to 4.

Y is —(CH₂)ₙ₃—,
wherein optionally one or more of each H in —(CH₂)ₙ₃— may be replaced by -(C₁-C₁₀)alkyl;

n³ is an integer between 1 to 10, preferably 1 to 6, more preferably 1 to 4.

In a further embodiment, the compound according to formula (I) is selected from the group consisting of

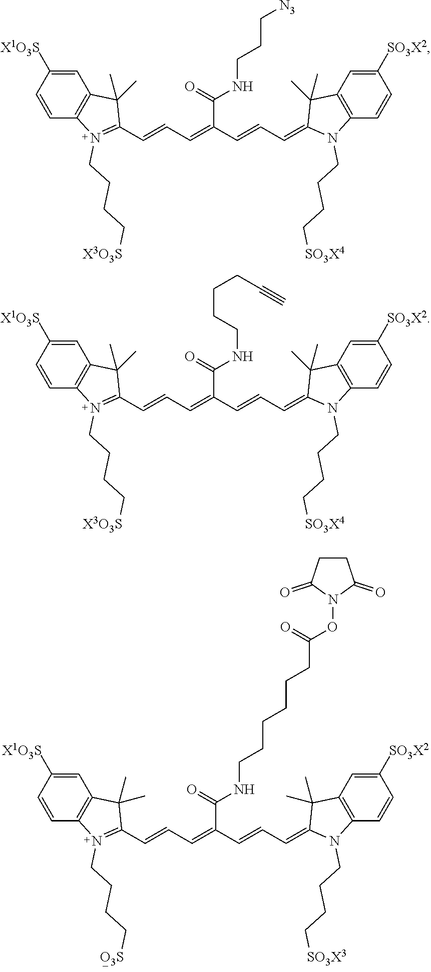

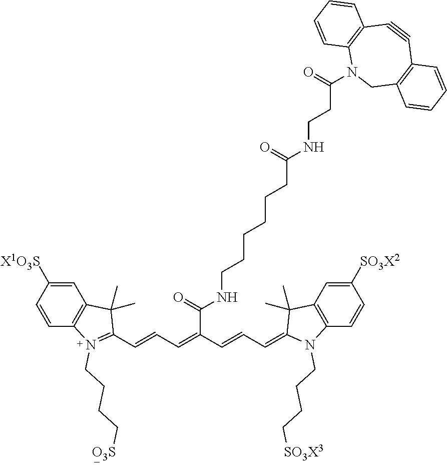

X¹, X², X³, and X⁴ are independently selected from a cation, a proton or absent, preferably the cation is a monovatent cation or a divalent cation, more preferably the cation is a monovalent cation most preferably an alkali metal cation or an ammonium cation, particularly preferred the cation is independently selected from the group consisting of Na⁺, K⁺; and NH₄⁺.

The compounds according to formula (I) may be generally prepared by following the approach of S̆tacková et al. by opening of Zincke Salts (3). An approach for the synthesis of compounds according to formula (I) is also outlined in more detail in the examples.

The compounds and dyes according to formula (I) may be further linked via a suitable linker, known to the person skilled in the art, for example with a biological active molecule such a's protein, peptide, small molecule, antibody, oligonucleotide, nanobody, or polymer, resulting in compounds according to formula (II).

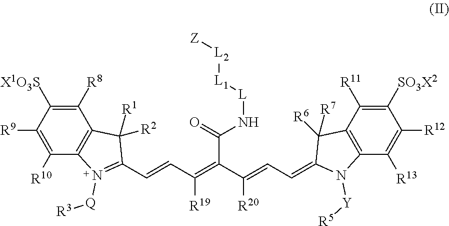

R¹, R², R⁶, R⁷, R¹⁴, R¹⁵, R¹⁶, R¹⁷, and R¹⁸ are independently selected from the group consisting of H and (C₁-C₆)alkyl, preferably methyl.

R³ is —SO₃X³, or COOH.

Z is a protein, peptide, small molecule, antibody, oligonucleotide, nanobody, mono- or polyvalent saccharide or polymer, preferably a protein, a peptide, a small molecule, an antibody or a nanobody, preferably a protein, a peptide, an antibody or a nanobody.

Z may be an antibody such a's Panitumumab (CAS-Nummer: 339177-26-3).

The protein or peptide may comprise proteinogenic amino acids and non-proteinogenic amino acids. Preferably the protein comprises only proteinogenic amino acids, selected from the group consisting of alanine, arginine, asparagine, asparaginic acid, cysteine, glutamine, glutaminic acid, glycine, histidine, isoleucine, leucine, lysine, methionine, phenylalanine, proline, serine, threonine, tryptophane, tryrosine, and valine. The peptide may be cyclic or acyclic.

Preferably, the protein has a molecular weight from 5000 Da to 400000 Da, more preferably 50000 to 300000 Da. Preferably, the protein is selected from the group consisting of leptin, insulin.

Preferably, the peptide comprises 2 to 45 amino acids, more preferably 2 to 20 amino acids a's described above. Preferably, the peptide is selected from the group consisting of Octreotide, Octreotate, nerve binding peptide 41 (NP41), glucagon and derivatives thereof, glucagon like peptide 1 (GLP-1) and derivatives thereof like eGLP-1, Exendin-4, lixisenatide, octreotide analogues TOC ([Tyr³]Octreotide), and TATE ([Tyr³, Thr⁸]Octreotide), methanobactin OB3b.

A small molecule is a molecule with a molecular weight below 900 g/mol, preferably a molecule that exhibits a pharmaceutical activity. Preferably, the small molecule is selected from the group consisting of prostate-specific membrane antigen (PSMA), PSMA binding ligands like Lysine-urea-glutamate (KUE),

An antibody also known a's an immunoglobulin, is a large, Y-shaped protein used by the immune system to identify and neutralize foreign objects such a's pathogenic bacteria and viruses. The antibody recognizes a unique molecule of the pathogen, called an antigen. Preferably, the antibody has a molecular weight of 10000 Da to 400000 Da, more preferably 50000 to 300000 Da. Preferably, the antibody is selected from the group consisting of EGFR targeting antibodies such a's cetuximab, and panitutumab, HER2 targeting antibodies such a's trastuzumab, carcino-embryonic antigen (CEA) targeting antibodies, and vascular endothelial growth factor receptor (VEGFR) targeting antibodies such a's bevatuzumab.

An oligonucleotide is a short single or double stranded DNA or RNA molecule of preferably 10-100 nucleotides, such a's an aptamer, small interfering RNA (siRNA), or short hairpin RNA (shRNA).

Nanobodies are antibodies which comprise just one domain compared to a regular antibody. This domain usually corresponds to the variable region of a conventional antibody. Nanobodies preferably have a size of 10 to 30 kDA, more preferably 12 to 20 kDa.

Saccharides are carbohydrates preferably mannose, glucose, galactose, N-acetyl galactosamine, galactosamine, gluconic acid, glucuronic acid or sialic acid, which can be monovalent or polyvalent, preferably coupled to each other by glycosidic bonds or employing a Tris(hydroxymethyl)aminomethane (TRIS) residue.

The Polymer may be any polymer known to the person skilled in the art which suitable for solving the require task, preferably oligosaccharide based polymers like dextran, chitosan, pullulan, alginate, lentinane, hyaluronic acid and combinations thereof, poly-lactic acid, poly-lysine, polyurethanes and polyhydroxyethylmethacrylates.

R⁵ is —SO₃X⁴.

R⁸, R⁹, R¹⁰, R¹¹, R¹² and R¹³ are independently selected from the group consisting of H, CN, —COO(C₁-C₆)alkyl, —CONH(C₁-C₆)alkyl, Cl, Br, I, F, —CF₃, —O(C₁-C₆), —NR¹⁴R¹⁵, —NHC(O)(C₁-C₆)alkyl, —NHC(O)NH(C₁-C₆)alkyl), —SO₃H, —SO₂NH₂, -(C₁-C₆)alkyl, preferably H.

R¹⁹, R²⁰ are independently selected from the group consisting of H, -(C₁-C₆)alkyl, —O(C₁-C₆)alkyl, Cl, F, Br, CN; C(O)NH₂, preferably H.

R²¹ is H, -(C₁-C₁₀)alkyl.

X¹, X², X³, and X⁴ are independently selected from a cation, a proton or absent, preferably the cation is a monovalent cation or a divalent cation, more preferably the cation is a monovalent cation most preferably an alkali metal cation or an ammonium cation, particularly preferred the cation is independently selected from the group consisting of Na⁺, K⁺; and NH₄⁺.

Q is —(CH₂)ₙ₂—,
wherein optionally one or more of each H in —(CH₂)ₙ₂— may be replaced by -(C₁-C₁₀)alkyl.

n² is an integer between 1 to 10, preferably 1 to 6, more preferably 1 to 4.

Y is —(CH₂)ₙ₃—, wherein optionally one or more of each H in —(CH₂)ₙ₂— may be replaced by -(C₁-C₁₀)alkyl.

n³ is an integer between 1 to 10, preferably 1 to 6, more preferably 1 to 4.

L and L₂ are each independently a group consisting of one or more and/or one or more of each of the elements selected from the group: —(CH₂)ₙ₁—, -(C₁-C₆)cycloalkylene-, -(C₁-C₆)heterocycloalkylene-, —O—, —NR¹⁶—, —NH—, —NHC(O)—, —C(O)NR¹⁷—, —CH(OH)—, and —CH(OR¹⁸)—.

Preferably L and L₂ are independently selected from the group consisting of CH₂(CH₂OCH₂)ₙ₁CH₂—, —(CH₂)ₙ₂—, —CH₂(CH₂NHCH₂)ₙ₁CH₂—, —CH₂(CH₂NR¹⁶CH₂)ₙ₁CH₂—, —CH₂(CH₂OCH₂)ₙ₁CH₂C(O)(CH₂)ₙ₅—, —CH₂(CH₂NHCH₂)ₙ₁CH₂C(O)(CH₂)ₙ₅—, —CH₂(CH₂NR¹⁶CH₂)ₙ₁CH₂C(O)(CH₂)ₙ₅—.

n¹ is an integer between 1 to 30, preferably 1 to 20, more preferably 1 to 10.

n² is an integer between 1 to 10, preferably 1 to 6, more preferably 1 to 4.

n⁵ is an integer between 1 to 10, preferably 1 to 6, more preferably 1 to 4.

n¹ is an integer between 1 to 30, preferably 1 to 20, more preferably 1 to 10.

Ld1 is a suitable linking moiety. Preferably, L₁ is selected from the group consisting of,

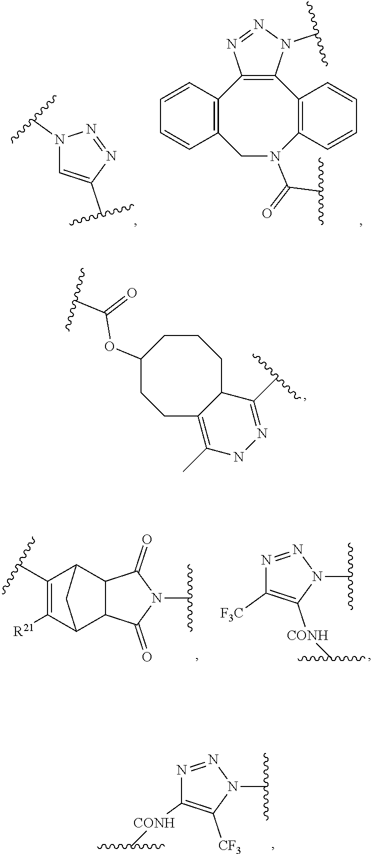

and —CO—, preferably

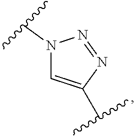 [SMILES: *c1cn(*)nn1]

### —CO—.

R²¹ is H, -(C₁-C₁₀)alkyl.

In a further embodiment, in formula (II)

R¹, R², R⁶, R⁷ are Methyl.

R³ is —SO₃X³.

R⁵ is —SO₃X⁴.

R⁸, R⁹, R¹⁰, R¹¹, R¹² and R¹³ are H.

R¹⁶, R¹⁷, and R¹⁸ are independently selected from the group consisting of H and (C₁-C₆)alkyl, preferably methyl.

R¹⁹ and R²⁰ are H.

X¹, X², X³, and X⁴ are independently selected from a cation, a proton or absent, preferably the cation is a monovalent cation or a divalent cation, more preferably the cation is a monovalent cation most preferably an alkali metal cation or an ammonium cation, particularly preferred the cation is independently selected from the group consisting of Na⁺, K⁺; and NH₄⁺.

Q is —(CH₂)ₙ₂—,
wherein optionally one or more of each H in —(CH₂)ₙ₂— may be replaced by -(C₁-C₁₀)alkyl.

n² is an integer between 1 to 10, preferably 1 to 6, more preferably 1 to 4.

Y is —(CH₂)ₙ₃—,
wherein optionally one or more of each H in —(CH₂)ₙ₃— may be replaced by -(C₁-C₁₀)alkyl.

n³ is an integer between 1 to 10, preferably 1 to 6, more preferably 1 to 4.

L and L₂ are each independently a group consisting of one or more and/or one or more of each of the elements selected from the group: —(CH₂)ₙ₁—, -(C₁-C₆)cycloalkylene-, -(C₁-C₆)heterocycloalkylene-, —O—, —NR¹⁶—, —NH—, —NHC(O)—, —C(O)NR¹⁷—, —CH(OH)—, and —CH(OR¹⁸)—.

Preferably L and L₂ are independently selected from the group consisting of CH₂(CH₂OCH₂)ₙ₁CH₂—, —(CH₂)ₙ₂—, —CH₂(CH₂NHCH₂)ₙ₁CH₂—, —CH₂(CH₂NR¹⁶CH₂)ₙ₁CH₂—, —CH₂(CH₂OCH₂)ₙ₁CH₂C(O)(CH₂)ₙ₅—, —CH₂(CH₂NHCH₂)ₙ₁CH₂C(O)(CH₂)ₙ₅—, —CH₂(CH₂NR¹⁶CH₂)ₙ₁CH₂C(O)(CH₂)ₙ₅—.

n¹ is an integer between 1 to 30, preferably 1 to 20, more preferably 1 to 10.

n⁴ is an integer between 1 to 10, preferably 1 to 6, more preferably 1 to 4.

L₁ is selected from the group consisting of

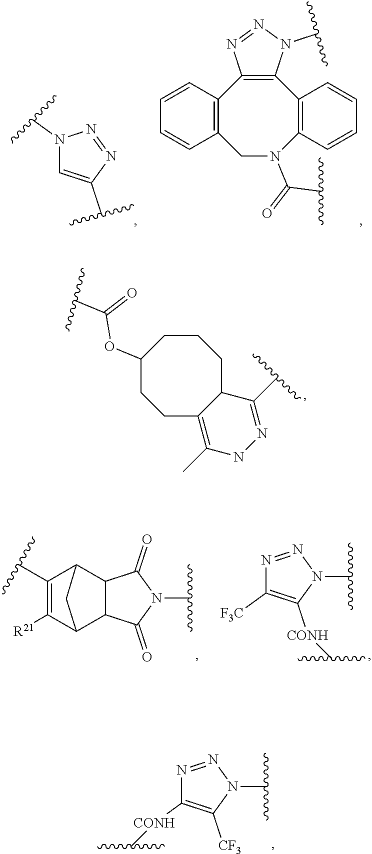

### —CO—,

R²¹ is H, -(C₁-C₁₀)alkyl;

In a further embodiment, the compound according to formula (II) is selected from the group consisting of

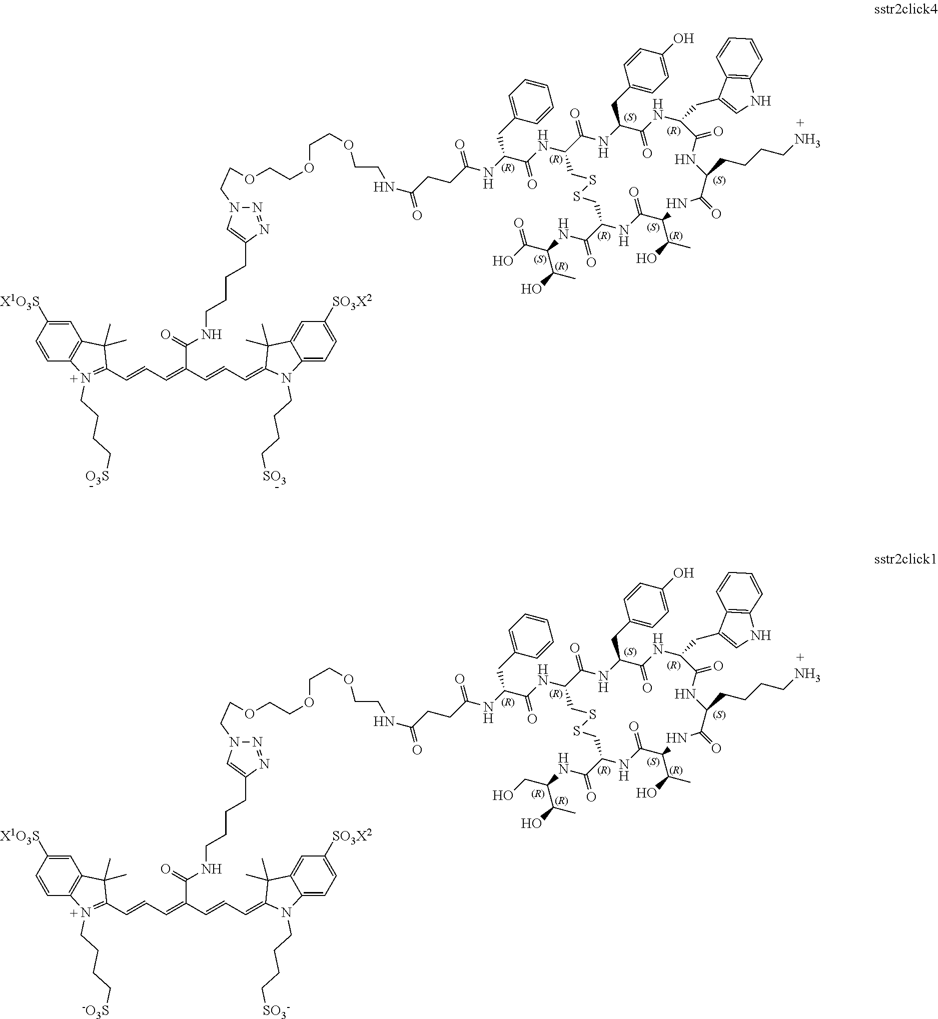

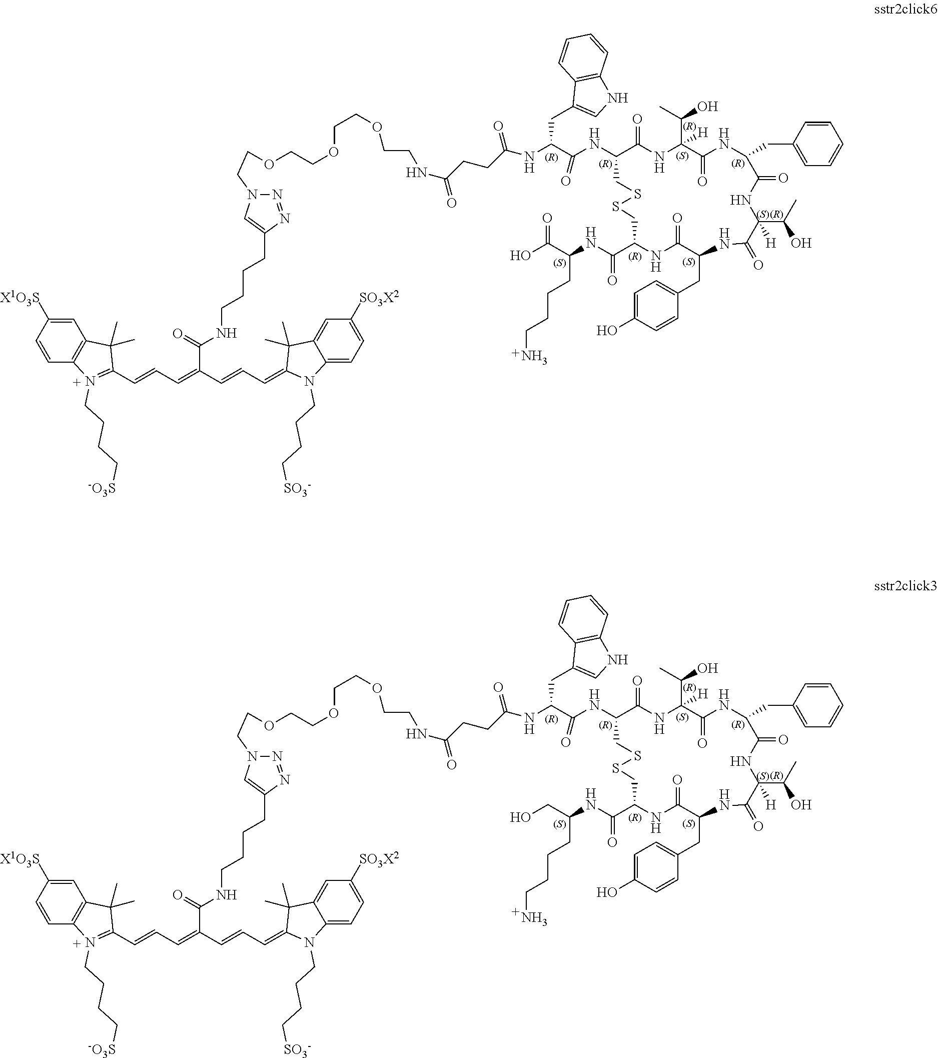

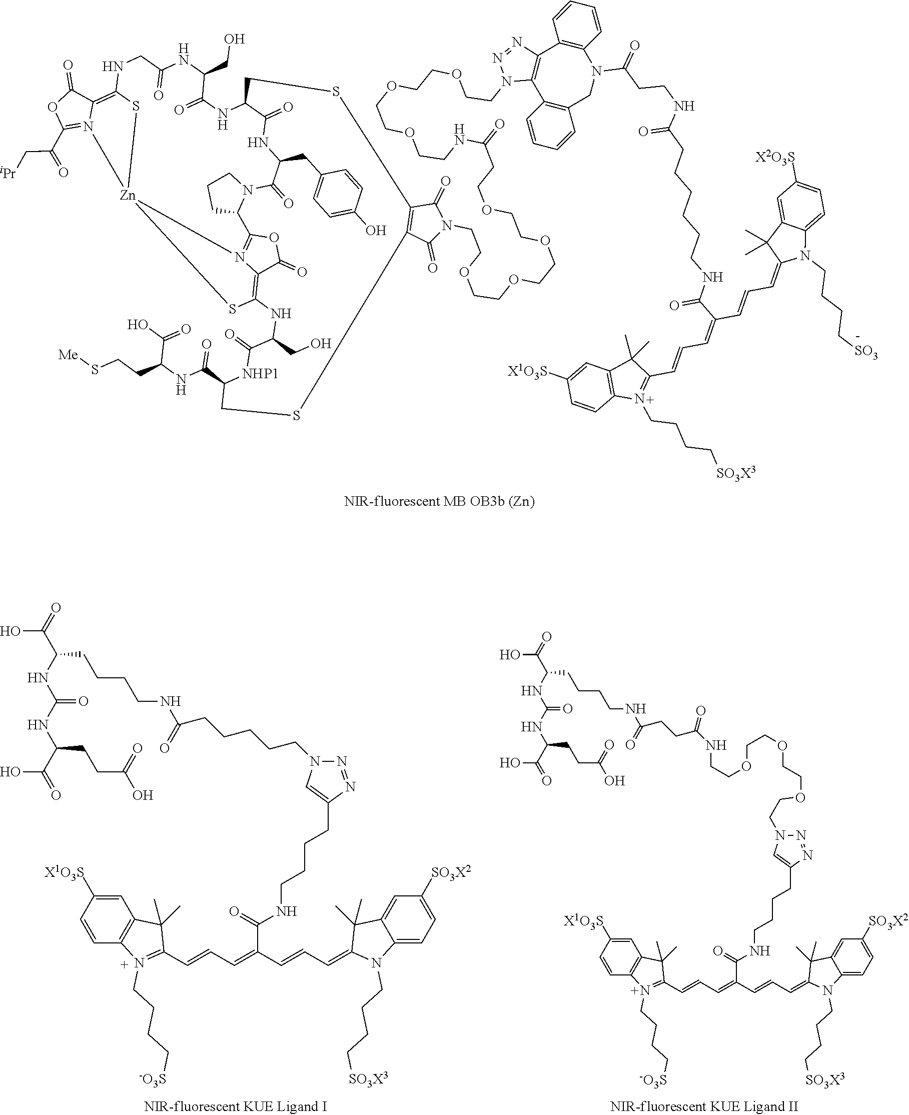

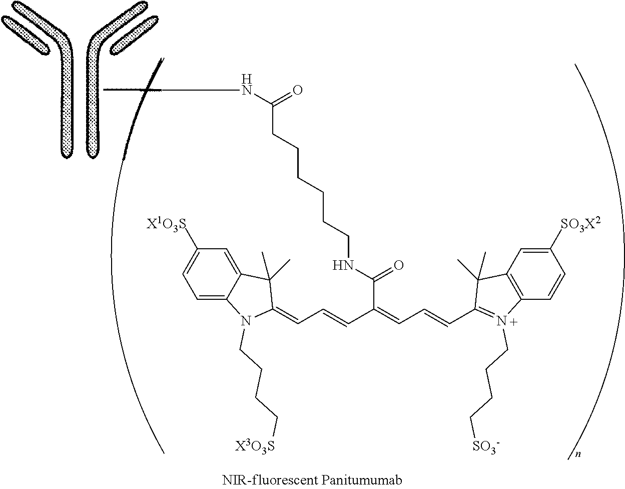

Wherein the antibody shown symbolically in the formula is Panitumumab.

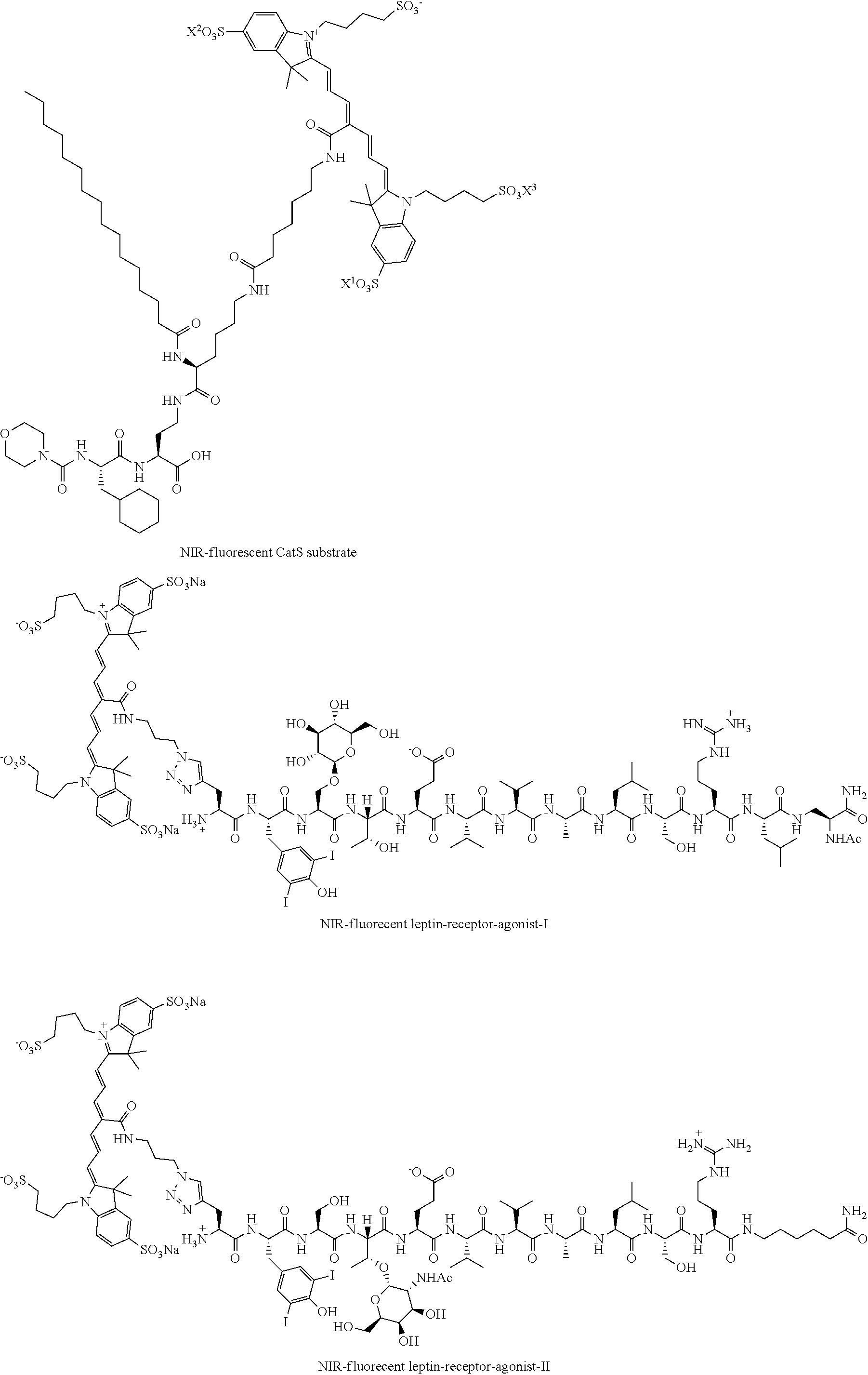

### Applications

The compounds of the present invention a's described above, preferably according to formula (I) may be used a's fluorescent dye.

Preferably, the peak emission in PBS of the compounds a's described above is between 400 nm to 1200 nm, more preferably from 500 nm to 900 nm, most preferably from 790 to 820 nm.

Preferably, the absorption crosssection in PBS of the compounds a's described above is >5,000 1/(M*cm), more preferably >50,000 1/(M*cm), most preferably >200,000 1/(M*cm).

Preferably, the peak absorption in PBS of the compounds a's described above is from 400 nm to 1200 nm, more preferably from 600 nm to 1000 nm, most preferably from 750 nm to 850 nm.

Moreover, the compounds of the present invention a's described above, preferably according to formula (II) may be used in vivo or ex vivo preferably in vivo a's contrast agent. Thus, the invention is also directed to the compounds according to formula (I) or (II) preferably according to formula (II) for use a's contrast agent in vivo.

Further, the compounds a's described above, preferably according to formula (II), are for use in diagnosis or for intraoperative surgery. Preferably, wherein the indication to be diagnosed or treated by intraoperative surgery is selected from the group consisting of inflammatory diseases, infectious diseases and cancer.

Preferably, the inflammatory disease is selected from the group consisting of otitis media, allergic rhinitis and chronic rhinosinusitis, chronic inflammation of tonsils, adenoids, and laryngitis.

Preferably, the infectious disease is selected from the group consisting of implant infection (e.g. hip, dental), secondary infections of the inner ear, e.g. accompanying cholesteatoma. This comprises infections caused by gram positive or gram negative bacteria, responding to antibiotic treatment, resistant or multi-drug resistant, in particular by ESKAPE strains Enterococcus faecium, Staphylococcus aureus, Klebsiella pneumoniae, Acinetobacter baumannii, Pseudomonas aeruginosa, and Enterobacter spp..

Preferably, diagnosing of cancer or treating cancers by intraoperative surgery relates to visualizing the primary tumor and/or metastatic lesions in situ during surgery selected from the group meningioma, glioblastoma, NSCLC, and pancreatic cancer.

A better understanding of the present invention and of its advantages will be had from the following examples, offered for illustrative purposes only. The examples are not intended to limit the scope of the present invention in any way.

### Examples of the Invention

### 1.1 Material und Methods

### General Notes

Solvents and reagents were purchased from ACROS, ALFA AESAR, CARL ROTH, COMBI-BLOCKS, ENAMINE, FISHER SCIENTIFIC, HONEYWELL, MERCK, SIGMA ALDRICH, TCl CHEMICALS and VWR and used a's received. Moisture- and air-sensitive reactions were performed in oven-dried glassware under argon atmosphere with anhydrous solvents. For oxygen-sensitive reactions, the solvent was degassed by passing an argon stream for ten minutes or by freeze-pump-thaw method. Reactions with light-sensitive substances were carried out in the absence of light a's far a's possible. Aqueous solutions used were based on demineralized water and were saturated unless otherwise described.

Solvent was removed under reduced pressure with a BÜCHI Rotavapor with a VACUUBRAND CVC 3000 vacuum pump. Aqueous solvent was removed by lyophilization with a CHRIST Alpha 1-4 LDplus Freeze Dryer with a VACUUBRAND RZ6 vacuum pump. Bath sonication was performed using a BANDELIN Sonorex.

### Thin Layer Chromatography (TLC)

Thin layer chromatography was performed using MERK'S silica gel-coated glass plates with fluorescence indicator DC Kieselgel 60 F₂₅₄. Visualization was performed using UV light (λ=254 or 366 nm) and/or by treating the plate with suitable stain (potassium permanganate, ceric ammonium molybdate, ninhydrin and bromcresol green) and subsequent heating.

### Column Chromatography

Flash column Chromatography was performed on a BÜCHI Reveleris® Prep system with UV and evaporative light scattering detector at variable flow rate. Silica, amino-, C8- and C18-modified silica cartridges manufactured by BÜCHI, AXEL SEMRAU and MACHERY-NAGEL were used. The particle size was 40-63 μm irregular or 20-40 μm spherical. Used solvents and gradients are listed in 1.2 Synthetic Procedures.

### NMR Spectroscopy

¹H and ¹³C NMR spectroscopic measurements were performed on BRUKER'S Ascend-400, Avance-400, Avance-500 and Avance-600 at 25° C. Calibration was performed to the residual proton signal of the deuterated solvent. The chemical shift δ is given in parts per million (ppm) and the coupling constant J in Hz. For the multiplicities the following abbreviations are used: singlet (s), doublet (d), triplet (t), quartet (q), quintet (quint.), broad singlet (bs), and multiplet (m). For a complete characterization, additional ¹H-¹H correlation spectra (COSY) and ¹H—¹³C correlation spectra (HMBC, HSQC) were acquired. The numbering of the carbon atoms corresponds to an arbitrary determination and does not follow the IUPAC rules.

### Infrared Spectroscopy

IR spectra were acquired on a SHIMADZU IRAffinity-1S.

### Mass Spectrometry

Analytical LC-MS measurements of molecules with M<1000 g/mol were performed using a WATERS Acquity UPLC H-Class System utilizing a QDa Mass Detector, a PDA eA Detector and a UPLC BEH C18-column (50×2.1 mm, particle size: 1.7 μm). Runs (05-95_3.5 min) employed a standard gradient a's listed in Table 1 at a flow rate of 0.8 mL/min. Electrospray ionization (ESI) was used and M/z range of 50-1250 was detected. WATERS MassLynx 4.1 software was used for analysis.

TABLE 1
Solvent gradient used for WATERS
Acquity UPLC H-Class System.

| time [min] | H₂O + 0.1% FA | MeCN + 0.1% FA |
|---|---|---|
| 0.00 | 95% | 5% |
| 1.00 | 5% | 95% |
| 2.25 | 5% | 95% |
| 2.27 | 95% | 5% |
| 3.50 | 95% | 5% |

Analytical LC-MS measurements of molecules with M>1000 g/mol were performed using a WATERS 2795 Separation Module connected to a WATERS LCT Premier Mass Spectrometer, a HP 1050 UV multiwavelength detector and a PHENOMENEX Aeris™ Widepore XB-C18 (50×2.1 mm, particle size: 3.6 μm) column. Runs (01-90_5 min) employed a standard gradient a's listed in Table 2 at a flow rate of 0.6 mL/min. Electrospray ionization (ESI) was used for ionization and M/z range of 100-2500 was detected. WATERS MassLynx 4.1 software was used for analysis.

TABLE 2
Solvent gradient used for composite HPLC system.

| time [min] | H₂O + 0.1% FA | MeCN + 0.1% FA |
|---|---|---|
| 0.00 | 99% | 1% |
| 4.00 | 10% | 90% |
| 5.00 | 10% | 90% |

Alternatively, analytical LC-MS measurements of molecules with M>1000 g/mol were performed using a THERMO FISHER SCIENTIFIC UltiMate 3000 UHPLC System utilizing a ISQ EC Mass Spectrometer, a Variable Wavelength Detector and a MACHERY-NAGEL Nucleodur PFP column (50×2.0 mm, particle size: 3.0 μm) with the corresponding precolumn cartridge. Runs (05-95_13 min) followed on an equilibration (5 min) and employed a standard gradient a's listed in Table 3 at a flow rate of 0.3 mL/min. Electrospray ionization (ESI) was used for ionization and M/z range of 100-1250 was detected. THERMO FISHER SCIENTIFIC'S Chromeleon CDS software was used for analysis.

TABLE 3
Solvent gradient used for the THERMO FISHER
SCIENTIFIC UltiMate 30000 UHPLC System.

| time [min] | H₂O + 0.1% FA | MeCN + 0.1% FA |
|---|---|---|
| −5 | 5% | 95% |
| 0 | 5% | 95% |
| 8 | 95% | 5% |
| 13 | 95% | 5% |

### Microwave Assisted Synthesis

Reactions under microwave irradiation were performed using the BIOTAGE Initiator+.

### Solid Phase Peptide Synthesis

Peptide synthesis was either performed automatically or manually. (Amino) acids used are listed in Table 4. L-amino acids are labeled with capital and D-amino acids with small letters.

TABLE 4
Overview of (amino) acids used for solid phase peptide synthesis.

| | Three/single letter | |
|---|---|---|
| (Amino) acid | code | Reagent |
| Alanine | Ala/A | Fmoc-Ala-OH |
| Arginine | Arg/R | Fmoc-Arg(Pbf)-OH |
| Asparagine | Asn/N | Fmoc-Asn(Trt)-OH |
| Aspartic acid | Asp/D | Fmoc-Arg(OtBu)-OH |
| Cysteine | Cys/C | Fmoc-Cys(Trt)-OH |
| Glutamine | Gln/Q | Fmoc-Gln(Trt)-OH |
| Glutamic acid | Glu/E | Fmoc-Glu(OtBu)-OH |
| Glycine | Gly/G | Fmoc-Gly-OH |
| Histidine | His/H | Fmoc-His(Trt)-OH |
| Isoleucine | Ile/I | Fmoc-Ile-OH |
| Leucine | Leu/L | Fmoc-Leu-OH |
| Lysine | Lys/K | Fmoc-Lys(Boc)-OH |
| Methionine | Met/M | Fmoc-Met-OH |
| Phenylalanine | Phe/F | Fmoc-Phe-OH |
| Proline | Pro/P | Fmoc-Pro-OH |
| Serine | Ser/S | Fmoc-Ser(tBu)-OH |
| Threonine | Thr/T | Fmoc-Thr(tBu)-OH |
| Tryptophan | Trp/W | Fmoc-Trp(Boc)-OH |
| Tyrosine | Tyr/Y | Fmoc-Tyr(tBu)-OH |
| Valine | Val/V | Fmoc-Val-OH |
| Propargylglycine | Pra/— | Fmoc-Pra-OH |
| Threonol | — | Fmoc-Thr(tBu)-ol |
| Lysinol | — | Fmoc-Lys(Boc)-ol |
| 6-Azidohexanoic acid | — | — |
| N₃-PEG(3)-CO₂H | — | — |

### Automated SSPS

Automated peptide synthesis was performed on a CEM Liberty Blue™ Automated Mirowave Peptide Synthesizer following a standard Fmoc protocol at 0.1 mmol scale. MERCK'S Rink Amide AM resin LL (100 200 mesh; loading 0.40 mmol/g), BACHEM'S 2-chlorotrityl chloride resin (200-400 mesh, loading 1.5-1.9 mmol/g) and MERCK'S DHP HM resin (100-200 mesh, 1.17 mmol/g) were used a's solid support. In some cases, manually preloaded resin were used. Therefore, the following procedure was followed:
2-chlorotrityl chloride resin
   Swelling (CH₂Cl₂, 0.5 h)
   Activation (dest. AcCl (1 mL/g resin), toluene, 60° C., 3 h, Ar)
   Loading (2.0 eq protected aminoacid, 5.0 eq dest. DIPEA, CH₂Cl₂, rt, 2.0 h)
   2× Capping (5 mL CH₂Cl₂/MeOH/DIPEA (8/1.5/0.5), rt, 0.5 h)
   Drying to constant mass in order to determine the loading efficacy
DHP HM resin
   Swelling (DCE, 1 h)
   Loading (3.5 eq protected aminoalcohol, 1.8 eq PPTS, DCE, 80° C., on, Ar)
   Capping (addition of pyridine, 15 min, rt)
   Drying to constant mass in order to determine the loading efficacy

Stock solutions of (amino) acids (0.2 M in DMF), DIC (0.5 M in DMF), Oxyma® (1 M in DMF), and a deprotection solution (10% (w/v) piperazine in EtOH:NMP (1:9) or 10% (v/v) piperidine in DMF) were prepared and provided to the instrument. Couplings were performed with a fivefold excess (compared to the resin) of (amino) acids a's well a's coupling and deprotection reagents. Instrument settings for deprotection and coupling are summarized in Table 5.

TABLE 5
Overview of the deprotection and coupling settings
used for automated peptide synthesis.

| | temperature | performance | hold time | ΔT |
|---|---|---|---|---|
| name | [° C.] | [W] | [s] | [° C.] |
| standard deprotection | 75 | 155 | 15 | 2 |
| | 85 | 30 | 50 | 1 |
| conventional | 25 | 0 | 900 | 2 |
| deprotection | | | | |
| standard coupling | 75 | 160 | 15 | 2 |
| | 90 | 3 | 110 | 1 |
| conventional coupling | 25 | 2 | 3600 | 2 |
| 50° C. coupling | 25 | 0 | 120 | 2 |
| | 50 | 35 | 240 | 1 |

### Manual SSPS

Manual peptide synthesis was performed following a standard Fmoc protocol at a 0.3 mmol scale. BACHEM'S 2-chlorotrityl chloride resin (200-400 mesh, loading 1.5-1.9 mmol/g) and MERCK'S DHP HM resin (100-200 mesh, 1.17 mmol/g) were used a's solid support. To a suspension of the resin (200 mg) in CH₂Cl₂ (2 mL) was added pyridine (0.1 mL) and SO₂Cl₂ (0.05 mL) at 0° C. The mixture was heated to reflux for 4 h without using a stirring bar, cooled to rt and then filtered using a 20 mL syringe reactor (equipped with a filter). The activated resin was washed with CH₂Cl₂ (3×4 mL) and dried. The resin was transferred into a 10 mL flask and CH₂Cl₂ (4 mL), the C-terminal amino alcohol (0.6 mmol, 2 equiv.) and distilled DIPEA (0.2 mL) were added. The flask was flushed with argon, closed with a glass stopper, placed into a beaker and shaken with 300 rpm on HEIDOLPH'S Rotamax 120 at rt for 18 h. The mixture was transferred into a 20 mL syringe reactor and the resin was washed with DMF (3×4 mL) and CH₂Cl₂ (3×4 mL).
For deprotection 20% piperidine in DMF (4 mL) were drawn into the syringe reactor and the mixture was shaken with 300 rpm on HEIDOLPH'S Rotamax 120 at rt for 15 min. The supernatant was filtered off and the deproctection procedure was performed a second time.
In the meanwhile, coupling was prepared by dissolving the following amino acid (1.2 mmol, 4 equiv.) in DMF/CH₂Cl₂ (1/1, v/v, 4 mL) in a 10 mL flask. HATU (456 mg, 1.2 mmol, 4 equiv.) and DIPEA (0.4 mL, 2.4 mmol, 8 equiv.) were added and the reaction mixture was stirred at rt for 20 min which resulted in a yellow coloration of the solution. The stirring bar was removed and the washed and dried resin was added to the flask. The flask was flushed with argon, closed with a glass stopper, placed into a beaker and shaken with 300 rpm on HEIDOLPH'S Rotamax 120 at rt for 1-18 h. The mixture was transferred into a 20 mL syringe reactor and the resin was washed with DMF (3×4 mL), CH2Cl2 (3×4 mL).

All following (amino) acids were coupled following the iterative sequence of deprotection, and coupling.

After completed (automated or manual) peptide synthesis, the suspension was transferred to a 20 mL syringe reactor. The resin-bound peptide was filtered and washed with DMF (3×5 mL) and CH₂Cl₂ (3×5 mL). For resin-cleavage and removal of the protecting groups, the resin-bound peptide was incubated with 10 mL TFA/H₂O/DODT/TIS (94%/2.5%/2.5%/1%, v/v/v/v) at rt and 300 rpm for a minimum of 4 h. The solution was filtered off and portioned onto two 40 mL Greiner tubes filled with 35 mL of ice cold diethyl ether. The tubes were centrifuged (4000 rpm, 4° C., 4 min), the supernatants discarded and the precipitates washed three times with diethylether. The two precipitates were dissolved in MeCN, combined, diluted with H₂O and lyophilized. Further purification is described in in 1.2 Synthetic Procedures.

### Absorbance and Emission Measurements

Quartz cuvettes (HELLMA 115B-QS, HELLMA 105.202-QS) were used for absorbance and emission measurements. All spectra were obtained at ambient temperature. Normalized spectra are displayed for clarity.

Absorbance spectra were obtained at a PERKIN ELMER Lambda 1050+ with solvent in the reference beam path. Spectrophotometer operated by PERKIN ELMER'S UV WinLab software.

Emission spectra were recorded using custom-built PRINCETON INSTRUMENT'S system. Emission in the visible wavelength range was detected with a Pixis:400BR camera (cooled to −70° C.) attached to a SpectraPro HRS-300-SS spectrograph. Emission in the Near and Shortwave Infrared wavelength range was detected with a Pylon-IR:1024 line-camera (cooled to −100° C.) attached to the same spectrograph. A grating with density 150 g/nm, a blaze of 800 nm was used. PRINCETON INSTRUMENTS LightField software was used to control acquisition.

Excitation wavelength were 635 nm (THORLABS CPS635R with THORLABS'S FL-635-10 clean-up filter) and 785 nm (THORLABS Fiber-Coupled Laser S4FC785 with THORLABS FL780-10 clean-up filter). For the excitation wavelengths, THORLABS FELH0650 and THORLABS FELH0800 were used a's emission filters, respectively.

The exposure times for both detectors were set to 1000 ms. Five frames per detector per spectrum were acquired. The frames were background-, flatfiled-corrected and averaged. The sum of the rows of frames acquired with the Pixis:400BR was taken. The resulting spectra were intensity corrected (THORLABS SLS201L/M). The intensity corrected spectra from both detectors were stitched resulting in the final spectrum.

### Imaging

Imaging was performed on a custom-built setup. LUMICS laser unit LU0785DLU250-S70AN03 (25 W) 785 nm″ was used for excitation. Laser was externally controlled using a LUMICS laser diode control system. Laser output was coupled in a 4×1 fan-out fiber-optic bundle (THORLABS BF46LSO1) of 600 μm core diameter for each optical path. The output from the fiber was fixed in an excitation cube (THORLABS KCB1EC/M), reflected off of a mirror (THORLABS BBE1-E03), and passed through a positive achromat (THORLABS AC254-050-B), SP filter (THORLABS FESH800) and an engineered diffuser (THORLABS ED1-S20-MD or ED1-S50-MD) to provide uniform illumination over the working area. In a typical experiment, the excitation flux in the field of view was adjusted to be close to 100 mWcm⁻². Power density was determined using a THORLABS energy meter console PM100D in combination with a THORLABS S130C sensor (measurement uncertainty of ±3%). The working area was covered by blackout fabric (THORLABS BK5). Emitted light was directed through two filters (THORLABS FELH800) screwed onto a EDMUND OPTICS Acktar Hexa-Black 25 mm noise reduction extension tube, via a C-mount camera lens (NAVITAR SWIR-35) onto an NIR-camera (PCO Edge 4.2 or Prime BSI). The software μManager was used to externally control the camera. The assembly was partially enclosed to avoid excess light while enabling manipulation of the field of view during operation.

### Procedure for Determining Photostability

For each dye (conjugate), solutions in H₂O and FBS with an OD (785 nm)<1.0 in a 1 cm cuvette (HELLMA 115B-QS) were prepared. Solutions were transferred to HIRSCHMANN mini caps (Code-Nr: 9000110, V=10 μL, d=0.0652 cm) and closed with wax. The inclusion of bubbles was avoided. Filled capillaries were placed on a homemade capillary holder made from acryl. Samples were continuously irradiated using the imaging setup described above. Data was acquired with 1 fps until samples were completely bleached. Images were processed using the software Fiji (ImageJ). All images were dark subtracted and corrected with a flatfield, that itself was dark-corrected, Gaussian-blurred and normalized to the maximum. In order to be able to make useful comparison between different samples and experiments, the time axis was converted into a number of absorbance events by multiplication with the “absorptions per molecule per second”.

### Imaging In Vivo

### Procedure for Measuring Tissue and Blood Clearance

The anesthetized mice were injected with 5 nmol of each dye (conjugate) dissolved in PBS under continuous imaging in a custom build imaging setup for one hour. For excitation a 785 nm laser (Fa. Lumics) with a power density of 50 mW/cm², an engineered diffuser (ED1-S20-MD, Fa. Thorlabs) and an 800 nm shortpass filter (FESH 800, Fa. Thorlabs) was used. On the emission site an 825-50 bandpass filter (Fa. Edmund Optics) and two 800 longpass filters (FELH 800, Fa. Thorlabs) were mounted in a zoom lens system (Optem Fusion Zoom lens, Fa. Qioptic) to optimize the signal for the used dyes and images were acquired using MicroManager and a NIR camera (pco edge 4.2, Fa. PCO). To reduce the bleaching effect, the laser was triggered by the camera so that it was turned on only during image acquisition every 10 to 20 s. The vital parameters of the mice were monitored during the continuous imaging, so that the anesthesia and temperature could be adjusted to the mice needs. In the end of the experiment the mice were sacrificed and after necropsy the organs were imaged to estimate biodistribution. Image processing (dark and flatfield correction) was performed in image j, then a ROI (region of interest) was drawn on the tissue of the front limb and the tail vain respectively, by using the “plot Z-axis profile” the mean value of the ROI was measured in all of the images. The blood clearance was corrected for the tissue clearance by subtracting the values of a ROI on the tail next to the tail vein.

### Bleaching In Vivo

Mice were injected with 5For image acquisition the same custom built camera setup was used a's mentioned above. For this experiment a second laser beam with the same 785 nm excitation but without engineered diffusor and shortpass filter was added to the setup. For this bleaching laser beam the power density was increased to 200-450 mW/cm² to cause a quick, continuous bleaching in a small area, here we chose one ear, kidney and leg for bleaching. The bleaching was stopped, when a steady state of the signal in the bleaching area was reached. Whole mouse images were acquired before and after the bleaching under the same conditions mentioned above with the bleaching laser beamed turned off. In the end of the experiment the mice were sacrificed and after necropsy, organs were imaged. Image processing was performed a's already mentioned and the bleaching kinetic was measured using the “plot Z-axis profile” tool for the ROI in the bleached area.

### 1.2 Synthetic Procedures

### 1.2.1 Conjugateable Heptamethine Cyanine Dyes

### 2,4-dinitrophenyltrifluoromethanesulfonate (zi1)

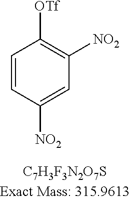 [SMILES: O=[N+]([O-])c1ccc(OS(=O)(=O)C(F)(F)F)c([N+](=O)[O-])c1]

2,4-dinitrophenol (sb1, 70% in H₂O, 800 mg, 3.04 mmol, 1.00 equiv.) and distilled triethylamine (422 μL, 3.04 mmol, 1.00 equiv.) were dissolved in CH₂Cl₂ (12.2 mL, 0.25 M). The solution was cooled to 0° C. and Tf₂O (1.02 mL, 6.08 mmol, 2.00 equiv.) was added. The reaction mixture was stirred at 0° C. for 20 min. The mixture was diluted with CH₂Cl₂ (10 mL), quenched with saturated aqueous NaHCO₃ (10 mL) and extracted with CH₂Cl₂ (3×100 mL). The combined organic layers were dried over Na₂SO₄, filtered and evaporated onto silica gel. Purification by chromatography (silica, 0%-20% Ethyl acetate/PE, main separation step at 2%) afforded triflate zi1 (718 mg, 2.27 mmol, 75%) a's a yellow oil. The analytical data are in agreement with those reported in the literature (15).

R_{f} (50% Ethyl acetate/PE)=0.75; ¹H NMR (400 MHz, CDCl₃): δ=9.03 (d, J=2.7 Hz, 1H), 8.62 (dd, J=9.0, 2.7 Hz, 1H), 7.76-7.69 (m, 1H) ppm; ¹³C NMR (101 MHz, CDCl₃): δ=146.6, 145.1, 129.6, 125.7, 122.5, 120.1, 117.0 ppm.

### N-(hex-5-yn-1-yl)isonicotinamide (zi2)

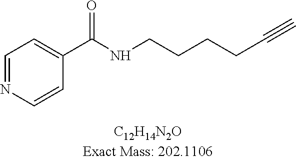

To an oven-dried two-neck flask was added isonicotinic acid (sb2, 750 mg, 6.10 mmol, 1.00 equiv.), a cat. amount of dry DMF (five drops) and dry CH₂Cl₂ (12.2 mL, 0.5 M) under argon. The solution was cooled to 0° C., oxalylchloride (627 μL, 7.32 mmol, 1.20 equiv.) was added dropwise and the reaction mixture was stirred at rt for 1 h. The volatiles were removed at reduced pressure and the residue was dissolved in CH₂Cl₂ (30.5 mL, 0.2 M). The solution was cooled to 0° C. and hexynamine (826 μL, 7.32 mmol, 1.20 equiv.) and distilled triethylamine (2.55 mL, 18.3 mmol, 3.00 equiv.) were added. After 5 min, the ice bath was removed and stirring was continued at rt for 1 h. The reaction mixture was quenched by the addition of saturated aqueous NaHCO₃ (5 mL), washed with saturated aqueous NaHCO₃ (25 mL) and extracted with CH₂Cl₂ (3×20 mL). The combined organic layers were dried over Na₂SO₄, filtered, concentrated under reduced pressure, and the crude was purified by column chromatography (silica, 0%-2% MeOH/CH₂Cl₂, main separation step at 2%). Alkyne zi2 (1.16 g, 5.72 mmol, 93%) was obtained a's a brown oil (16).

R_{f} (10% MeOH/CH₂Cl₂+0.5% TEA)=0.6; ¹H NMR (400 MHz, CDCl₃): δ=8.61 (d, J=5.9 Hz, 2H), 7.58 (dd, J=4.5, 1.6 Hz, 2H), 7.16 (s, 1H), 3.44-3.36 (m, 2H), 2.17 (td, J=6.9, 2.6 Hz, 2H), 1.92 (t, J=2.7 Hz, 1H), 1.69 (ddd, J=14.6, 9.8, 7.1 Hz, 2H), 1.55 (ddd, J=10.6, 7.2, 2.2 Hz, 2H) ppm; ¹³C NMR (101 MHz, CDCl₃): δ=165.8, 150.3, 141.9, 121.1, 83.9, 69.0, 39.7, 28.4, 25.7, 18.1 ppm; LC-MS (05-95_3.5 min): m/z 203 [M+H+]⁺, t_{R}=0.99 min; HRMS (ESI+): calculated for C₁₂H₁₅N₂O⁺[M+H⁺]+ 203.1184, observed 203.1186.

### 1-(2,4-dinitrophenyl)-4-(hex-5-yn-1-ylcarbamoyl)pyridin-1-iumtriflat (zi3)

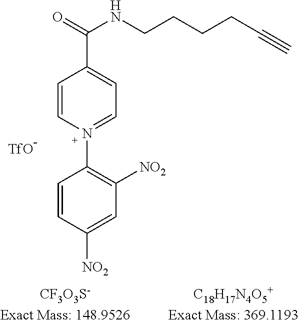

Triflate zi1 (172 mg, 0.54 mmol, 1.10 equiv.) and alkyne zi2 (100 mg, 0.49 mmol, 1.00 equiv.) were dissolved in toluene (3.5 mL, 0.15 M). The reaction mixture was refluxed for 16 h and left to cool to rt. The resulting precipitate was filtered, washed with toluene (5 mL) and Et2O (5 mL) and dissolved in MeOH (10 mL). The solvent was removed under reduced pressure. Zincke salt zi3 (258 mg, 0.49 mmol, quant.) was obtained a's a brown solid and used without any further purification.

R_{f} (20% MeOH/CH₂Cl₂)=0.7; ¹H NMR (400 MHz, CD₃OD): δ=9.44-9.41 (m, 2H), 9.29 (d, J=2.5 Hz, 1H), 8.93 (dd, J=8.7, 2.5 Hz, 1H), 8.67-8.63 (m, 2H), 8.32-8.28 (m, 1H), 3.57-3.49 (m, 2H), 2.31-2.21 (m, 3H), 1.88-1.77 (m, 2H), 1.70-1.60 (m, 2H) ppm; ¹³C NMR (101 MHz, CD3OD): δ=163.6, 153.4, 151.3, 148.4, 144.5, 139.9, 132.5, 131.2, 127.2, 123.2, 84.6, 69.9, 41.2, 29.2, 27.0, 18.7 ppm; LC-MS (05-95_3.5 min): m/z 369 [M-OTf-]⁺, t_{R}=1.01 min; HRMS (ESI+): calculated for C₁₈H₁₇N₄O₅⁺ [M-OTf-]⁺ 369.1187, observed 369.1199.

### 3-azidopropan-1-amine hydrochloride (zi4)

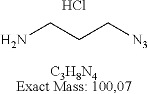

To a solution of 3-azidopropan-1-amine hydro bromide (sb4, 1.00 g, 4.57 mmol, 1.00 equiv.) in water (3.1 mL, 0.15 M) was added aqueous NaN₃ (3.3 M, 6.9 mL, 23.1 mmol, 5.00 equiv.). The reaction mixture was refluxed for 20 h, left to cool to rt, basified with aqueous KOH (10%, 10 mL) and extracted with Et₂O (3×20 mL). The combined organic layers were washed with aqueous HCl (1 M, 3×20 mL). The combined aqueous layers were lyophilized to afford azide zi4 (449 mg, 3.29 mmol, 72%) a's a white solid, which was used without any further purification.

¹H NMR (400 MHz, D₂O) δ=3.52 (t, J=6.5 Hz, 2H), 3.11 (t, J=7.5 Hz, 2H), 2.00-1.92 (m, 2H) ppm; ¹³C NMR (101 MHz, D₂O) b 48.2, 37.3, 26.1 ppm; HRMS (ESI+): calculated for C₃H₁₀N₄⁺ [M+H⁺]+ 101.0822, observed 101.0815; IR: \tilde{v} [cm⁻¹]=2094.69 (azide).

### N-(3-azidopropyl)isonicotinamide (zi5)

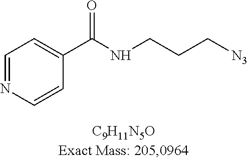 [SMILES: [N-]=[N+]=NCCCNC(=O)c1ccncc1]

To a solution of azide zi4 (449 mg, 3.30 mmol, 1.00 equiv.) in dry CH₂Cl₂/DMF (1/1, 16.5 mL, 0.2 M) were added isonicotinic acid (sb2, 406 mg, 3.30 mmol, 1.00 equiv.), EDC-HCl (696 mg, 3.64 mmol, 1.10 equiv.) and NaHCO₃ (1.38 g, 16.5 mmol, 5.00 equiv.). The suspension was stirred at rt for 16 h. The volatiles were removed at reduced pressure and the residue was diluted with saturated aqueous NaHCO₃ (10 mL) and extracted with Et₂O (3×20 mL). The combined organic layers were washed with brine (30 mL), dried over Na₂SO₄, filtered and evaporated onto silica gel. Purification by chromatography (silica, 0%-10% MeOH/CH₂Cl₂, main separation step at 3%) afforded azide zi5 (620 mg, 3.03 mmol, 92%) a's a brown solid.

R_{f} (10% MeOH/CH₂Cl₂)=0.4, ¹H NMR (400 MHz, CDCl₃) δ=8.70-8.64 (m, 2H), 7.63-7.57 (m, 2H), 3.53 (q, J=6.4 Hz, 2H), 3.43 (t, J=6.4 Hz, 2H), 1.88 (p, J=6.5 Hz, 2H) ppm; ¹³C NMR (101 MHz, CDCl₃) δ=165.9, 150.5, 141.7, 121.1, 49.6, 38.1, 28.6 ppm; LC-MS (05-95_3.5 min): m/z 206 [M+H⁺]⁺, t_{R}=0.70 min; HRMS (ESI+): calculated for C₉H₁₁N₅ONa⁺ [M+Na⁺]⁺ 228.0862, observed 228.0861; IR: \tilde{ν} [cm⁻¹]=2090.84 (azide).

### 4-((3-azidopropyl)carbamoyl)-1-(2,4-dinitrophenyl)pyridin-1-ium triflate (zi6)

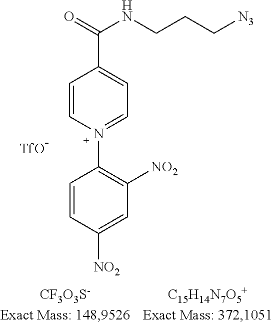

Triflate zi1 (804 mg, 2.54 mmol, 1.10 equiv.) and azide zi5 (475 mg, 2.31 mmol, 1.00 equiv.) were dissolved in toluene (21 mL, 0.15 M). The reaction mixture was refluxed for 18 h and left to cool to rt. The resulting precipitate was filtered, washed with toluene (10 mL) and Et₂O (10 mL) and dissolved in MeOH (20 mL). The solvent was removed under reduced pressure. Zincke salt zi6 (1.14 g, 2.20 mmol, 86%) was Exact Mass: 148,9526 Exact Mass: 372,1051 obtained a's a brown solid and used without any further purification.

¹H NMR (400 MHz, CD₃OD) δ=9.46-9.40 (m, 2H), 9.29 (d, J=2.4 Hz, 1H), 8.93 (dd, J=8.7, 2.5 Hz, 1H), 8.69-8.63 (m, 2H), 8.30 (d, J=8.7 Hz, 1H), 3.59 (t, J=6.8 Hz, 2H), 3.49 (t, J=6.6 Hz, 2H), 1.96 (p, J=6.7 Hz, 2H) ppm; ¹³C NMR (101 MHz, CD₃OD) δ=163.8, 153.3, 151.3, 148.4, 144.5, 139.9, 132.5, 131.2, 127.3, 123.2, 50.2, 39.2, 29.4 ppm; LC-MS (05-95_3.5 min): m/z 372 [M-OTf⁻]⁺, t_{R}=0.71 min; HRMS (ESI+): calculated for C₁₅H₁₄N₇O₅⁺ [M-OTf⁻]⁺ 372.1051, observed 372.1056; IR: \tilde{ν} [cm⁻¹]=2098.55 (azide).

### Methyl 7-(isonicotinamido)heptanoate (zi7)

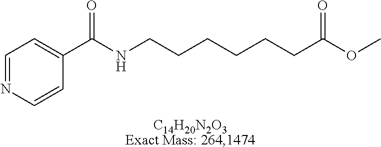 [SMILES: COC(=O)CCCCCCNC(=O)c1ccncc1 ; "C14H20N2O3Exact Mass: 264,1474"]

To an an oven-dried two-neck flask were added isonicotinic acid (sb2, 1.00 g, 8.13 mmol, 1.00 equiv.) a cat. amount of dry DMF (five drops) and dry CH₂Cl₂ (16.3 mL, 0.5 M). The solution was cooled to 0° C., oxalylchloride (0.84 mL, 9.75 mmol, 1.20 equiv.) was added dropwise and the reaction mixture was stirred at rt for 1 h. The volatiles were removed at reduced pressure and the residue was dissolved in CH₂Cl₂ (40.6 mL, 0.2 M). The solution was cooled to 0° C. and amine sb5 (2.22 mL, 9.75 mmol, 1.20 equiv.) and distilled triethylamine (3.39 mL, 18.3 mmol, 3.00 equiv.) were added. After 5 min, the ice bath was removed and stirring was continued at rt for 1 h. The reaction mixture was quenched by the addition of saturated aqueous NaHCO₃ (10 mL), washed with saturated aqueous NaHCO₃ (30 mL) and extracted with CH₂Cl₂ (3×40 mL). The combined organic layers were dried over Na₂SO₄, filtered, concentrated under reduced pressure, and the crude was purified by column chromatography (silica, 0%-2% MeOH/CH₂Cl₂, main separation step at 2%). Ester zi7 (1.88 g, 7.11 mmol, 87%) was obtained a's a brown oil.

R_{f} (10% MeOH/CH₂Cl₂+0.5% TEA)=0.25; ¹H NMR (400 MHz, CDCl₃) δ=8.75-8.68 (m, 2H), 7.64-7.55 (m, 2H), 3.48-3.42 (m, 2H), 2.31 (t, J=7.4 Hz, 2H), 1.68-1.57 (m, 4H), 1.44-1.31 (m, 4H) ppm; ¹³C NMR (101 MHz, CDCl₃) δ=174.3, 165.7, 150.7, 142.0, 121.0, 51.7, 40.2, 34.0, 29.4, 28.8, 26.6, 24.8 ppm; LC-MS (05-95_3.5 min): m/z 265 [M+H⁺]⁺, t_{R}=1.12; HRMS (ESI+): calculated for C₁₄H₂₀N₂O₃Na⁺ [M+Na⁺]⁺ 287.1372, observed 287.1386.

### 1-(2,4-dinitrophenyl)-4-((7-methoxy-7-oxoheptyl)carbamoyl)pyridin-1-ium triflate (zi8)

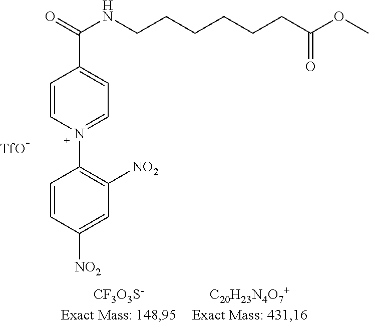

Triflate zi1 (864 mg, 2.73 mmol, 1.10 equiv.) und ester zi7 (656 mg, 2.48 mmol, 1.00 equiv.) were dissolved in toluene (21 mL, 0.15 M). The reaction mixture was refluxed for 18 h and left to cool to rt. The resulting precipitate was filtered, washed with toluene (10 mL) and Et₂O (10 mL) and dissolved in MeOH (20 mL). The solvent was removed under reduced pressure. Zincke salt zi8 (1.42 g, 2.73 mmol, 99%) was obtained a's a brown solid and used without any further purification.

¹H NMR (400 MHz, CD₃OD) δ=9.45-9.41 (m, 2H), 9.29 (d, J=2.4 Hz, 1H), 8.93 (dd, J=8.7, 2.5 Hz, 1H), 8.68-8.62 (m, 2H), 8.30 (d, J=8.7 Hz, 1H), 3.66 (s, 3H), 3.50 (t, J=7.2 Hz, 2H), 2.35 (t, J=7.4 Hz, 2H), 1.76-1.59 (m, 4H), 1.51-1.36 (m, 4H) ppm; ¹³C NMR (101 MHz, CD₃OD) δ=175.9, 163.6, 153.5, 151.3, 148.3, 144.5, 139.9, 132.5, 131.2, 127.2, 123.2, 52.0, 41.6, 34.7, 29.9, 29.8, 27.7, 25.9 ppm; LC-MS (05-95_3.5 min): m/z 431 [M-OTf⁻]⁺, t_{R}=1.08; HRMS (ESI+): calculated for C₂₀H₂₃N₄O₇⁺ [M-OTf⁻]⁺ 431.1567, observed 431.1516.

### 4-(2,3,3-trimethyl-3H-indol-1-ium-1-yl)butan-1-sulfonate (hv2)

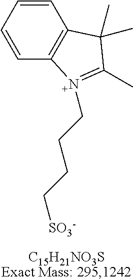

A solution of indole hve1 (1.00 g, 6.29 mmol, 1.00 equiv.) and 1,4-butane sultone (670 μL, 6.61 mmol, 1.05 equiv.) in toluene (10.5 mL, 0.6 M) was heated at 140° C. for 7 h in a microwave reactor. The reaction mixture was left to cool to rt and the supernatant was decanted into another microwave vial. The precipitate was washed with toluene (10 mL), petrolether (10 mL) and aceton (10 mL), dissolved in MeOH (20 mL) and concentrated under reduced pressure. Another portion 1,4-butane sultone (670 μL, 6.61 mmol, 1.05 equiv.) was added to the supernatant and the mixture was heated again at 140° C. for 7 h in a microwave reactor. The resulting precipitate was washed a's previously described, combined with the other precipitate and purified by column chromatography (C₁₈, 5%-95% MeCN+0.05% TFA/H₂O+0.05% TFA, main separation step at 5%). Heterocycle hv2 (931 mg, 3.16 mmol, 51%) was obtained a's an off-white solid. The analytical data are in agreement with those reported in the literature (17).

R_{f} (20% MeOH/CH₂Cl₂)=0.1; ¹H NMR (400 MHz, DMSO-d₆): δ=8.05-8.00 (m, 1H), 7.85-7.80 (m, 1H), 7.64-7.58 (m, 2H), 4.49 (t, J=7.9 Hz, 2H), 2.85 (s, 3H), 2.55-2.51 (m, 1H), 1.96 (q, J=7.7 Hz, 2H), 1.75 (q, J=7.5 Hz, 2H), 1.53 (s, 5H) ppm; ¹³C NMR (101 MHz, DMSO-d₆): δ=196.5, 141.6, 141.2, 129.4, 129.0, 123.4, 115.6, 74.4, 54.1, 50.2, 47.3, 26.0, 22.0, 13.9.ppm; LC-MS (05-95_3.5 min): m/z 296 [M+H⁺]⁺, t_{R}=0.68 min; HRMS (ESI+): calculated for C₁₅H₂₁NO₃SNa⁺ [M+Na⁺]⁺ 318.1140, observed 318.1132.

### 4-hydrazineylbenzenesulfonic acid (hv5)

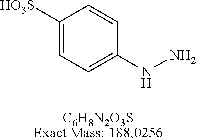 [SMILES: NNc1ccc(S(=O)(=O)O)cc1 ; "C6H8N2O3SExact Mass: 188,0256"]

In a two-neck flask 4-aminobenzenesulfonic acid (hve5, 3.00 g, 17.3 mmol, 1.00 equiv.) was suspended in H₂O (11.5 mL, 1.5 M). The suspension was heated to 85° C., followed by the portion wise addition of Na₂CO₃ (973 mg, 9.18 mmol, 0.53 equiv.). The reaction mixture was cooled to 5° C. and H₂SO₄ (1.15 mL, 1.25 mmol, 1.25 equiv.) was added dropwise. Aqueous NaNO₂ (6 M, 2.89 mL, 17.3 mmol, 1.00 equiv.) was added at 5° C., causing a brownish coloration of the solution and precipitation of a white solid. The diazo-compound was filtered, washed with H₂O, added to a solution of Na₂SO₃ (4.91 g, 39.0 mmol, 2.25 equiv.) in H₂O (14.4 mL, 2.7 M) at 5° C. and stirring at this temperature was continued for 1 h. The reaction mixture was heated to reflux followed by the addition of conc. HCl (11.6 mL, 141 mmol, 8.16 equiv.). The reaction mixture was left to cool to rt and the resulting precipitate was filtered and washed with H₂O (20 mL). Hydrazine hv5 (2.11 g, 11.2 mmol, 65%) was obtained a's a white solid and used without any further purification.

R_{f} (10% MeOH/CH₂Cl₂)=0.4; ¹H NMR (400 MHz, DMSO-d₆): δ=7.51 (d, J=8.3 Hz, 2H), 8.38 (d, J=6.6 Hz, 2H) ppm; ¹³C NMR (101 MHz, DMSO-d₆): δ=145.7, 141.4, 126.2, 113.0 ppm; HRMS (ESI−): calculated for C₆H₇N₂O₃S⁻ [M−H⁺]⁻ 187.0177, observed 187.0172.

### potassium 2,3,3-trimethyl-3H-indole-5-sulfonate (hv6)

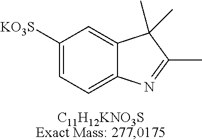

To a suspension of hydrazine hv5 (2.11 g, 11.2 mmol, 1.00 equiv.) in acetic acid (11.2 mL, 1 M) was added 3-methyl-2-butanone (3.6 mL, 33.7 mmol, 3.00 equiv.). The reaction mixture was refluxed for 3 h and then cooled to 5° C. Et₂O (10 mL) was added causing precipitation. The solid was filtered, washed with Et₂O (10 mL), dissolved in MeOH (20 mL) and concentrated under reduced pressure. The ammonium salt was dissolved in MeOH/iPrOH (1/1, 40 mL, 1 M), followed by the addition of grounded KOH (0.71 g, 12.7 mmol, 1.18 equiv.). Sulfonated indole hv6 (1.98 g, 7.2 mmol, 67%) was obtained a's a hygroscopic yellow solid and used without any further purification.

¹H NMR (400 MHz, D₂O): δ=7.91 (d, J=1.6 Hz, 1H), 7.85 (dd, 2H, J=8.7, 1.7 Hz, 1H), 7.57 (d, J=8.3 Hz, 1H), 1.39 (s, 6H) ppm; ¹³C NMR (101 MHz, D₂O): δ=197.1, 151.0, 145.8, 140.8, 125.9, 119.7, 118.3, 54.5, 21.6 ppm; LC-MS (05-95_3.5 min): m/z 240 [M−K⁺+2H⁺]⁺, t_{R}=0.45 min; HRMS (ESI−): calculated for C₁₁H₁₂NO₃⁻ [M−K⁺]⁻ 238.0538, observed 238.0529.

### potassium 2,3,3-trimethyl-1-(4-sulfonatobutyl)-3H-indol-1-ium-5-sulfonate (hv7)

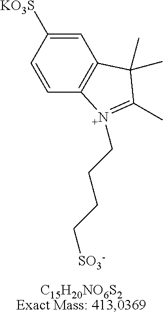

Sulfonated indole hv6 (2.20 g, 7.9 mmol, 1.00 equiv.) was suspended in 1,4-butane sultone (16.1 mL, 158.8 mmol, 20.0 Aq.) and heated to 120° C. The solution was stirred at this temperature for 12 h. The reaction mixture was left to cool to rt and the resulting precipitate was filtered, washed with Ethyl acetate (10 mL), dissolved in MeOH (20 mL) and concentrated under reduced pressure. Purification by column chromatography (C₈, 2%-95% MeCN+0.05% TFA/H₂O+0.05% TFA, main separation step at 2%) afforded heterocycle hv7 (1.14 g, 3.0 mmol, 38%) a's a gray-brown solid.

¹H NMR (400 MHz, DMSO-d₆): δ=8.01 (s, 1H), 7.96 (d, J=8.4 Hz, 1H), 7.81 (dd, J=8.4, 1.6 Hz, 1H), 4.46 (t, J=7.9 Hz, 2H), 2.84 (s, 3H), 2.53 (t, J=7.3 Hz, 2H), 1.95 (p, J=7.8 Hz, 2H), 1.75 (p, J=7.3 Hz, 2H), 1.54 (s, 6H) ppm; ¹³C NMR (101 MHz, DMSO-d₆): δ=197.2, 149.5, 141.5, 141.0, 126.3, 120.7, 115.0, 69.8, 54.2, 50.1, 26.0, 22.1, 21.91, 13.9 ppm; LC-MS (05-95_3.5 min): m/z 376 [M−K⁺+2H⁺]⁺, t_{R}=0.2 min; HRMS (ESI−): calculated for C₁₅H₂₀NO₆S₂—[M−K⁺]⁻ 374.0732, observed 374.0719.

### sodium 4-((E)-2-((2E,4Z,6E)-7-(3,3-dimethyl-1-(4-sulfonatobutyl)-3H-indol-1-ium-2-yl)-4-(hex-5-yn-1-ylcarbamoyl)hepta-2,4,6-trien-1-ylidene)-3,3-dimethylindolin-1-yl)butane-1-sulfonate (dyealkin2)

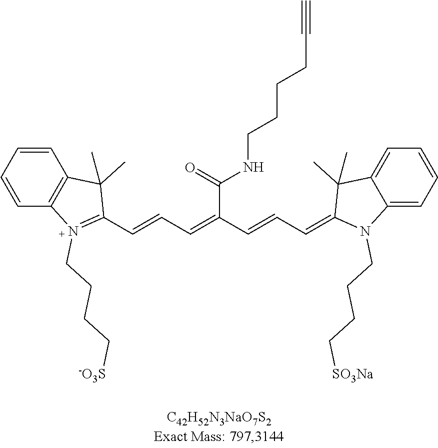

To a solution of Zincke salt zi3 (150 mg, 0.29 mmol, 1.00 equiv.) in MeOH (2.1 mL, 0.14 M) was added 4-bromoaniline (59.4 mg, 0.35 mmol, 1.20 equiv.). The reaction mixture was stirred at rt for 30 min. Heterocycle hv2 (256 mg, 0.87 mmol, 3.00 equiv.) and NaOAc (143 mg, 1.73 mmol, 6.00 equiv.) were added and the reaction mixture was stirred at rt for 1 h under light exclusion. Afterwards, Et₂O (6 mL) was added and the mixture was placed at −20° C. for 16 h. The resulting precipitate was filtered, washed with Et₂O (10 mL) and CH₂Cl₂ (10 mL), dissolved in MeOH (20 mL) and concentrated under reduced pressure. Purification by column chromatography (C18 gold, 5%-95% MeCN+0.05% TFA/H₂O+0.05% TFA, main separation step at 35%) afforded cyanine dyeaklin2 (147 mg, 0.18 mmol, 64%) a's a green solid.

R_{f} (20% MeOH/CH₂Cl₂)=0.08; ¹H NMR (400 MHz, DMSO-d₆): δ=8.92 (bt, J=5.9 Hz, 1H), 7.74-7.62 (m, 2H), 7.57 (d, J=7.4 Hz, 2H), 7.41 (dt, J=15.2, 7.9 Hz, 4H), 7.23 (t, J=7.3 Hz, 2H), 6.53 (d, J=13.6 Hz, 2H), 6.46 (d, J=13.2 Hz, 2H), 4.10 (t, J=7.6 Hz, 4H), 3.34 (q, J=6.8 Hz, 2H), 2.87 (d, J=2.6 Hz, 1H), 2.59 (t, J=7.1 Hz, 4H), 2.25 (td, J=7.0, 2.5 Hz, 2H), 1.85-1.63 (m, 10H), 1.56 (s, 14H) ppm; ¹³C NMR (101 MHz DMSO-d₆): δ=171.3, 166.8, 158.8, 146.4, 142.1, 141.0, 128.6, 124.9, 122.5, 121.8, 111.5, 104.7, 84.3, 71.7, 50.8, 48.7, 43.6, 38.6, 28.9, 27.5, 26.2, 25.6, 22.3, 17.6 ppm; LC-MS (05-95_3.5 min): m/z 777 [M−Na⁺+2H⁺]⁺, t_{R}=1.1 min; HRMS (ESI−): calculated for C₄₂H₅₂N₃O₇S₂— [M−Na⁺]⁻ 774.3247, observed 774.3256.

### sodium 2-((1E,3Z,5E)-7-((E)-3,3-dimethyl-5-sulfonato-1-(4-sulfonatobutyl)indolin-2-ylidene)-4-(hex-5-yn-1-ylcarbamoyl)hepta-1,3,5-trien-1-yl)-3,3-dimethyl-1-(4-sulfonatobutyl)-3H-indol-1-ium-5-sulfonate (dyealkin5)

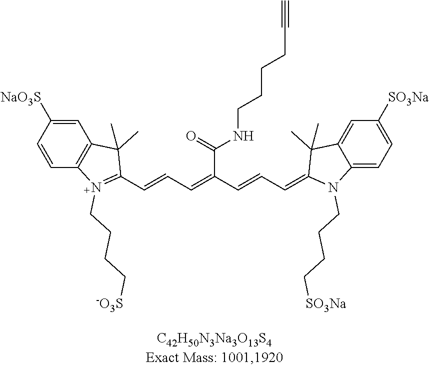

To a solution of Zincke salt zi3 (150 mg, 0.29 mmol, 1.00 equiv.) in MeOH (2.1 mL, 0.14 M) was added 4-bromoaniline (59.4 mg, 0.35 mmol, 1.20 equiv.). The reaction mixture was stirred at rt for 30 min. Heterocycle hv7 (326 mg, 0.87 mmol, 3.00 equiv.) and NaOAc (143 mg, 1.73 mmol, 6.00 equiv.) were added and the reaction mixture was stirred at rt for 1 h under light exclusion. Afterwards, Et₂O (6 mL) was added and the mixture was placed at −20° C. for 16 h. The resulting precipitate was filtered, washed with Et₂O (10 mL) and CH₂Cl₂ (10 mL), dissolved in MeOH (20 mL) and concentrated under reduced pressure. Purification by column chromatography (C18 gold, 5%-95% MeCN+0.05% TFA/H₂O+0.05% TFA, main separation step at 25%) afforded cyanine dyeaklin5 (88 mg, 0.09 mmol, 32%) a's a green solid.

R_{f} (20% MeOH/CH₂Cl₂)=0.00; ¹H NMR (400 MHz, D₂O): δ=7.83-7.70 (m, 4H), 7.63 (bd, J=11.1 Hz, 2H), 7.28 (d, J=8.2 Hz, 2H), 6.38 (bd, J=12.3 Hz, 2H), 3.98 (s, 4H), 3.47 (t, J=7.2 Hz, 2H), 2.89 (t, J=7.3 Hz, 5H), 2.40 (t, J=2.6 Hz, 1H), 2.27 (td, J=6.9, 2.6 Hz, 2H), 1.93-1.70 (m, 10H), 1.65-1.57 (m, 2H), 1.54 (s, 12H) ppm; ¹³C NMR (101 MHz, D₂O): δ=172.7, 169.5, 146.7, 144.2, 141.6, 139.0, 126.6, 122.8, 119.7, 111.1, 85.5, 69.8, 50.3, 49.0, 43.7, 39.5, 28.4, 27.1, 25.5, 25.3, 21.6, 17.4 ppm*; LC-MS (01-95_5 min): m/z 934 [M−2Na⁺]⁻, t_{R}=3.22 min; HRMS (ESI−): calculated for C₄₂H₅₀N₃Na₂O₁₃S₄⁻ [M−Na⁺]− 978.2022, observed 978.2070.
only one of three polyene carbon atoms visible in ¹³C NMR

### Sodium 4-(2-((1E,3Z,5E)-4-((3-Oss SO_{3N}a azidopropyl)carbamoyl)-7-((E)-3,3-dimethyl-1-(4-C₃₉H₄₉N₆NaO₇S₂ sulfonatobutyl)indolin-2-ylidene)hepta-1,3,5-trien-1-yl)-3,3-dimethyl-3H-indol-1-ium-1-yl)butane-1-sulfonate (dyeazid2)

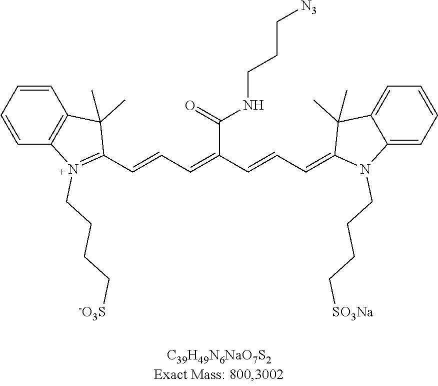

To a solution of Zincke salt zi6 (150 mg, 0.29 mmol, 1.00 equiv.) in MeOH (2.1 mL, 0.14 M) was added 4-bromoaniline (59.4 mg, 0.35 mmol, 1.20 equiv.). The reaction mixture was stirred at rt for 30 min. Heterocycle hv2 (255 mg, 0.87 mmol, 3.00 equiv.) and NaOAc (143 mg, 1.73 mmol, 6.00 equiv.) were added and the reaction mixture was stirred at rt for 1 h under light exclusion. Afterwards, Et₂O (6 mL) was added and the mixture was placed at −20° C. for 16 h. The resulting precipitate was filtered, washed with Et₂O (10 mL) and CH₂Cl₂ (10 mL), dissolved in MeOH (20 mL) and concentrated under reduced pressure. Purification by column chromatography (C18 gold, 5%-95% MeCN+0.05% TFA/H₂O+0.05% TFA, main separation step at 35%) afforded cyanine dyeazid2 (157 mg, 0.20 mmol, 68%) a's a green solid.

R_{f} (20% MeOH/CH₂Cl₂)=0.08; ¹H NMR (400 MHz, DMSO-d₆): δ=8.97 (bt, J=5.7 Hz, 1H), 7.68 (t, J=13.4 Hz, 2H), 7.56 (d, J=7.4 Hz, 2H), 7.41 (dt, J=15.3, 7.9 Hz, 4H), 7.24 (t, J=7.4 Hz, 2H), 6.54 (d, J=13.6 Hz, 2H), 6.48 (d, J=9.8 Hz, 2H), 4.11 (d, J=7.4 Hz, 4H), 3.49 (t, J=6.8 Hz, 2H), 3.41 (q, J=6.7 Hz, 2H), 2.60 (t, J=7.0 Hz, 4H), 1.90-1.67 (m, 11H), 1.55 (s, 14H) ppm; ¹³C NMR (101 MHz, DMSO): δ=171.3, 167.1, 158.3, 146.3, 142.1, 140.9, 128.6, 124.9, 122.4, 121.8, 111.5, 104.7, 50.8, 48.7, 48.5, 43.7, 36.4, 28.9, 27.5, 26.2, 22.2 ppm; LC-MS (05-95_3.5 min): m/z 780 [M−Na⁺+2H⁺]⁺, t_{R}=1.08 min; HRMS (ESI−): calculated for C₃₉H₄₉N₆O₇S₂⁻ [M−Na⁺]⁻ 777.3104, observed 777.3110.

### sodium 2-((1E,3Z,5E)-4-((3-azidopropyl)carbamoyl)-7-((E)-3,3-dimethyl-5-sulfonato-1-(4-sulfonatobutyl)indolin-2-ylidene)hepta-1,3,5-trien-1-yl)-3,3-dimethyl-1-(4-sulfonatobutyl)-3H-indol-1-ium-5-sulfonate (dyeazid3)

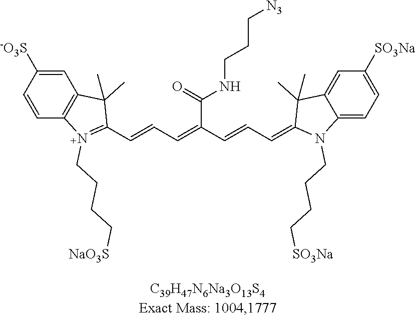

To a solution of Zincke salt zi6 (150 mg, 0.29 mmol, 1.00 equiv.) in MeOH (2.1 mL, 0.14 M) was added 4-bromoaniline (59.4 mg, 0.35 mmol, 1.20 equiv.). The reaction mixture was stirred at rt for 30 min. Heterocycle hv7 (326 mg, 0.87 mmol, 3.00 equiv.) and NaOAc (143 mg, 1.73 mmol, 6.00 equiv.) were added and the reaction mixture was stirred at rt for 1 h under light exclusion. Afterwards, Et₂O (6 mL) was added and the mixture was placed at −20° C. for 16 h. The resulting precipitate was filtered, washed with Et₂O (10 mL) and CH₂Cl₂ (10 mL), dissolved in MeOH (20 mL) and concentrated under reduced pressure. Purification by column chromatography (C18 gold, 5%-95% MeCN+0.05% TFA/H₂O+0.05% TFA, main separation step at 25%) afforded cyanine dyeazid3 (112 mg, 0.12 mmol, 41%) a's a green solid.

R_{f} (20% MeOH/CH₂Cl₂)=0.00; ¹H NMR (400 MHz, D₂O): δ=7.86-7.79 (m, 4H), 7.67 (t, J=13.2 Hz, 2H), 7.33 (d, J=8.4 Hz, 2H), 6.43 (d, J=13.3 Hz, 2H), 6.31 (d, J=13.5 Hz, 2H), 4.03 (s, 4H), 3.59 (t, J=7.0 Hz, 2H), 3.52 (t, J=6.7 Hz, 2H), 2.98-2.91 (m, 4H), 2.03-1.95 (m, 2H), 1.95-1.79 (m, 8H), 1.59 (s, 12H) ppm; ¹³C NMR (101 MHz, D₂O): δ=172.8, 169.6, 146.7, 144.1, 141.7, 139.2, 126.6, 123.1, 119.7, 117.8, 111.1, 105.4, 50.3, 49.0, 48.7, 43.8, 37.3, 28.4, 27.1, 25.6, 21.6 ppm; LC-MS (05-95_3.6 min): m/z 939 [M−3Na⁺+4H⁺]⁺, t_{R}=0.74 min; HRMS (ESI−): calculated for C₃₉H₄₈N₆NaO₁₃S₄²⁻ [M−2Na⁺]²⁻ 479.0991, observed 479.0990.

### sodium 4-((E)-2-((2E,4Z,6E)-7-(3,3-dimethyl-1-(4-sulfonatobutyl)-3H-indol-1-ium-2-yl)-4-((7-methoxy-7-oxoheptyl)carbamoyl)hepta-2,4,6-trien-1-ylidene)-3,3-dimethylindolin-1-yl)butane-1-sulfonate (dyecarb1)

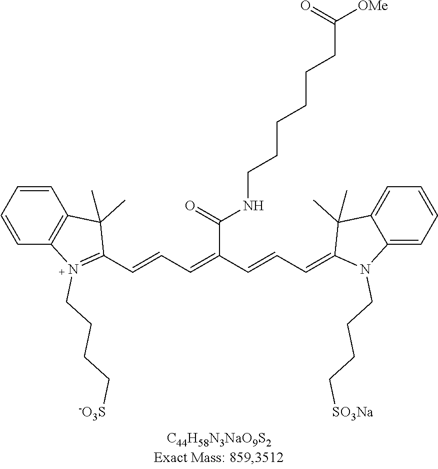

To a solution of Zincke salt zi8 (100 mg, 0.17 mmol, 1.00 equiv.) in MeOH (1.2 mL, 0.14 M) was added 4-bromoaniline (35.5 mg, 0.21 mmol, 1.20 equiv.). The reaction mixture was stirred at rt for 30 min. Heterocycle hv2 (153 mg, 0.52 mmol, 3.00 equiv.) and NaOAc (84.7 mg, 1.03 mmol, 6.00 equiv.) were added and the reaction mixture was stirred at rt for 1 h under light exclusion. Afterwards, Et₂O (4 mL) was added and the mixture was placed at −20° C. for 16 h. The resulting precipitate was filtered, washed with Et₂O (10 mL) and CH₂Cl₂ (10 mL), dissolved in MeOH (20 mL) and concentrated under reduced pressure. Purification by column chromatography (C18 gold, 5%-95% MeCN+0.05% TFA/H₂O+0.05% TFA, main separation step at 40%) afforded cyanine dyecarb1 (83 mg, 0.10 mmol, 56%) a's a green solid.

R_{f} (20% MeOH/CH₂Cl₂)=0.15; ¹H NMR (400 MHz, DMSO-d₆): δ=8.88 (t, J=5.7 Hz, 1H), 7.68 (t, J=13.4 Hz, 2H), 7.58 (dd, J=7.2, 1.0 Hz, 2H), 7.48-7.36 (m, 4H), 7.24 (td, J=7.3, 1.2 Hz, 2H), 6.53 (d, J=13.6 Hz, 2H), 6.46 (d, J=13.2 Hz, 2H), 4.11 (t, J=7.3 Hz, 4H), 3.58 (s, 3H), 3.31 (q, J=6.8 Hz, 2H), 2.53 (t, J=7.2 Hz, 4H), 2.34 (t, J=7.4 Hz, 2H), 1.84-1.66 (m, 8H), 1.55 (s, 14H), 1.41-1.31 (m, 4H) ppm; ¹³C NMR (101 MHz, DMSO-d₆): δ=173.4, 171.3, 166.8, 146.4, 142.1, 140.9, 128.6, 124.8, 122.4, 121.7, 111.4, 104.6, 51.2, 50.8, 48.6, 43.6, 39.0, 33.3, 29.5, 28.2, 27.4, 26.3, 26.2, 24.3, 22.4 ppm; LC-MS (05-95_3.5 min): m/z 838 [M−Na⁺+2H⁺]⁺, t_{R}=1.06 min; HRMS (ESI−): calculated for C₄₄H₅₈N₃O₉S₂⁻ [M−Na⁺]⁻ 837.3693, observed 837.3705.

### sodium 4-(2-((1E,3Z,5E)-4-((6-carboxyhexyl)carbamoyl)-7-((E)-3,3-dimethyl-1-(4-sulfonatobutyl)indolin-2-ylidene)hepta-1,3,5-trien-1-yl)-3,3-dimethyl-3H-indol-1-ium-1-yl)butane-1-sulfonate (dyecarb2)

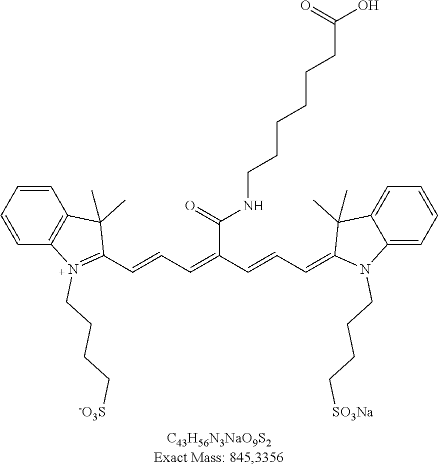

Dyecarb1 (18.5 mg, 21.5 μmol, 1.00 equiv.) was dissolved in THF/H₂O (3/1, v/v, 4.9 mL, 5 mM) under light exclusion. An aqueous solution of LiOH*H₂O (0.6 M, 0.3 mL, 172 μmol, 8.00 equiv.) was added to the reaction mixture, causing an orange coloration of the green solution. The reaction mixture was stirred at rt for 2 h and neutralized by the addition of acetic acid (57 μL, 172 μmol, 8.00 equiv.), which caused a green coloration. The volatiles were removed at reduced pressure, water (2 mL) was added and the solution was lyophilized. Purification by column chromatography (C18 gold, 5%-95% MeCN+0.05% TFA/H2O+0.05% TFA, main separation step at 35%) afforded cyanine dyecarb2 (9.10 mg, 10.8 μmol, 50%) a's a green solid.

LC-MS (05-95_3.5 min): m/z 823 [M−Na⁺+2H⁺]⁺, t_{R}=1.04 min; HRMS (ESI−): calculated for C₄₃H₅₆N₃O₉S₂⁻ [M−Na⁺]⁻ 822.3458, observed 822.3465.

### sodium 4-((E)-2-((2E,4Z,6E)-7-(3,3-dimethyl-1-(4-sulfonatobutyl)-3H-indol-1-ium-2-yl)-4-((7-((2,5-dioxopyrrolidin-1-yl)oxy)-7-oxoheptyl)carbamoyl)hepta-2,4,6-trien-1-ylidene)-3,3-dimethylindolin-1-yl)butane-1-sulfonate (dyecarb3)

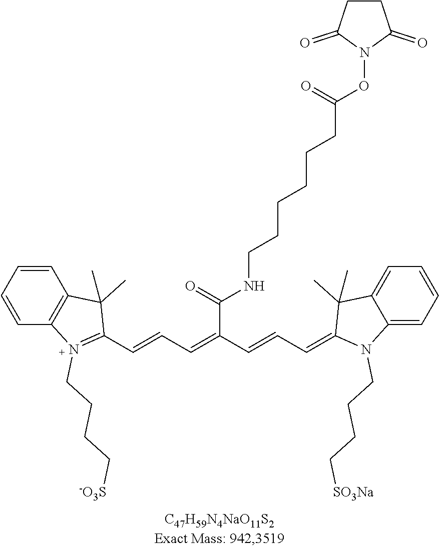

To a solution of dyecarb2 (5.7 mg, 6.74 μmol, 1.00 equiv.) in DMF (2.7 mL, 2.5 mM) were added DIPEA (4.6 μL, 27.0 μmol, 4.00 eqiv) and TSTU (8.1 mg, 27.0 μmol, 4.00 eqiv). The reaction was sonicated and stirred at rt for 1 h under light exclusion. The volatiles were removed at reduced pressure and the residue was diluted with H₂O/MeCN (4/1, v/v, 0.5 mL) and purified by column chromatography (C18 gold, 5%-95% MeCN+0.05% TFA/H2O+0.05% TFA, main separation step at 35%). Dyecarb3 (5.00 mg, 5.31 μmol, 79%) was obtained a's a green solid.

LC-MS (05-95_3.5 min): m/z 921 [M−Na⁺+2H⁺], t_{R}=1.08 min; HRMS (ESI−): calculated for C₄₇H₅₉N₄O₁₁S₂⁻ [M−Na⁺]⁻ 919.3622, observed 919.3660.

### sodium 2-((1E,3Z,5E)-7-((E)-3,3-dimethyl-5-sulfonato-1-(4-sulfonatobutyl)indolin-2-ylidene)-4-((7-methoxy-7-oxoheptyl)carbamoyl)hepta-1,3,5-trien-1-yl)-3,3-dimethyl-1-(4-sulfonatobutyl)-3H-indol-1-ium-5-sulfonate (dyecarb5)

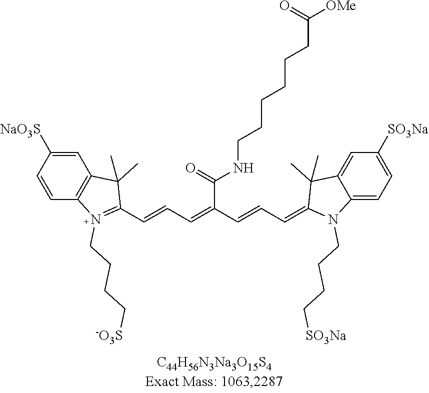

To a solution of Zincke salt zi8 (270 mg, 0.47 mmol, 1.00 equiv.) in MeOH (3.3 mL, 0.14 M) was added 4-bromoaniline (95.9 mg, 0.56 mmol, 1.20 equiv.). The reaction mixture was stirred at rt for 30 min. Heterocycle hv7 (553 mg, 1.39 mmol, 3.00 equiv.) and NaOAc (229 mg, 2.79 mmol, 6.00 equiv.) were added and the reaction mixture was stirred at rt for 1 h under light exclusion. Afterwards, Et₂O (15 mL) was added and the mixture was placed at −20° C. for 16 h. The resulting precipitate was filtered, washed with Et₂O (10 mL) and CH₂Cl₂ (10 mL), dissolved in MeOH (20 mL) and concentrated under reduced pressure. Purification by column chromatography (C18 gold, 1%-95% MeCN+0.05% TFA/H₂O+0.05% TFA, main separation step at 10%) afforded cyanine dyecarb5 (60.5 mg, 0.06 mmol, 13%) a's a green solid.

HRMS (ESI−): calculated for C₄₄H₅₆N₃NaO₁₅S₄²⁻ [M−2Na⁺]²⁻ 508.6246, observed 508.6247.

### sodium 2-((1E,3Z,5E)-4-((6-carboxyhexyl)carbamoyl)-7-((E)-3,3-dimethyl-5-sulfonato-1-(4-sulfonatobutyl)indolin-2-ylidene)hepta-1,3,5-trien-1-yl)-3,3-dimethyl-1-(4-sulfonatobutyl)-3H-indol-1-ium-5-sulfonate (dyecarb6)

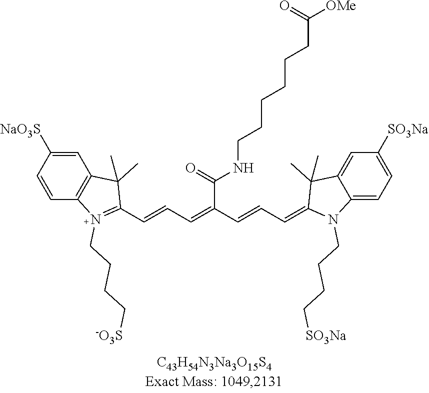

Dyecarb5 (30.0 mg, 28.2 μmol, 1.00 equiv.) was dissolved in THF/H₂O (3/1, v/v, 3.5 mL, 8 mM) under light exclusion. An aqueous solution of LiOH*H₂O (0.6 M, 0.47 mL, 282 μmol, 10.0 equiv.) was added to the reaction mixture, causing an orange coloration of the green solution. The reaction mixture was stirred at rt for 2 h and neutralized by the addition of acetic acid (57 μL, 282 μmol, 8.00 equiv.), which caused a green coloration. The volatiles were removed at reduced pressure, water (2 mL) was added and the solution was lyophilized. Purification by column chromatography (C18 gold, 5%-95% MeCN+0.05% TFA/H2O+0.05% TFA, main separation step at 10%) afforded cyanine dyecarb6 (21.8 mg, 20.8 μmol, 74%) a's a green solid.

LC-MS (05-95_3.5 min): m/z 982 [M−3Na⁺+2H⁺]⁻, t_{R}=1.07 min; HRMS (ESI−): calculated for C₄₄H₅₄N₃NaO₁₅S₄²⁻ [M−2Na⁺]²⁻ 501.6168, observed 501.6172.

### sodium 2-((1E,3Z,5E)-7-((E)-3,3-dimethyl-5-sulfonato-1-(4-sulfonatobutyl)indolin-2-ylidene)-4-((7-((2,5-dioxopyrrolidin-1-yl)oxy)-7-oxoheptyl)carbamoyl)hepta-1,3,5-trien-1-yl)-3,3-dimethyl-1-(4-sulfonatobutyl)-3H-indol-1-ium-5-sulfonate (dyecarb7)

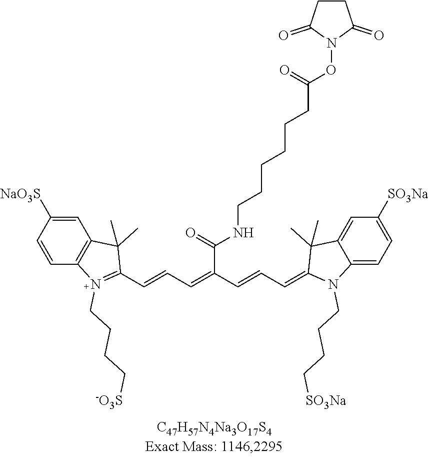

To a solution of dyecarb6 (21.5 mg, 20.0 μmol, 1.00 equiv.) in DMF (8.2 mL, 2.5 mM) were added DIPEA (13.9 μL, 820 μmol, 4.00 eqiv) and TSTU (24.7 mg, 82.0 μmol, 4.00 eqiv). The reaction was sonicated and stirred at rt for 1 h under light exclusion. The reaction mixture was inversely added to ice cold diethyl ether and the precipitate was purified by column chromatography (C18 gold, 5%-95% MeCN+0.05% TFA/H2O+0.05% TFA, main separation step at 15%). Dyecarb7 (19.0 mg, 17.0 μmol, 81%) was obtained a's a green solid.

HRMS (ESI−): calculated for C₄₇H₅₇N₄NaO₁₇S₄²⁻ [M−2Na⁺]²⁻ 550.1250, observed 550.1249.

### Dyecarb8

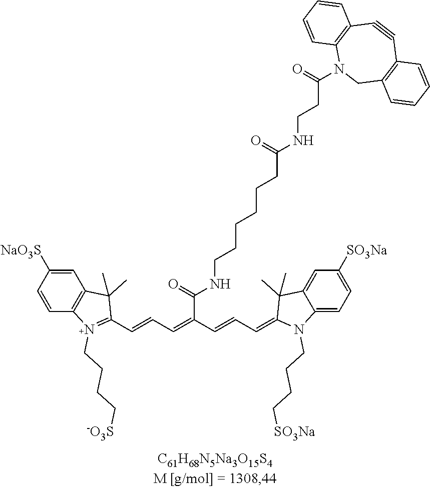

Dyecarb7 (8.00 mg, 6.98 μmol, 1.00 equiv.) was dissolved in DMSO/H₂O (1/1, v/v, 2.0 mL, 3.5 mM). DBCO-Amin (2.1 μL, 7.68 μmol, 1.10 equiv.) and DIPEA (5.9 μL, 34.9 μmol, 5.0 equiv.) were added and the reaction mixture was stirred at rt for 30 min. The reaction mixture was directly purified by column chromatography (C18 gold, 1%-95% MeCN+0.05% TFA/H₂O+0.05% TFA, main separation step at 30%). Cyanine dyecarb8 (3.00 mg, 2.42 μmol, 35%) was obtained a's a green solid.

### sodium 2-((E)-2-((E)-3-(2-((E)-3,3-dimethyl-5-sulfonato-1-(4-sulfonatobutyl)indolin-2-ylidene)ethylidene)-2-(4-sulfonatophenoxy)cyclohex-1-en-1-yl)vinyl)-1-(6-(hex-5-yn-1-ylamino)-6-oxohexyl)-3,3-dimethyl-3H-indol-1-ium-5-sulfonate (dye1)

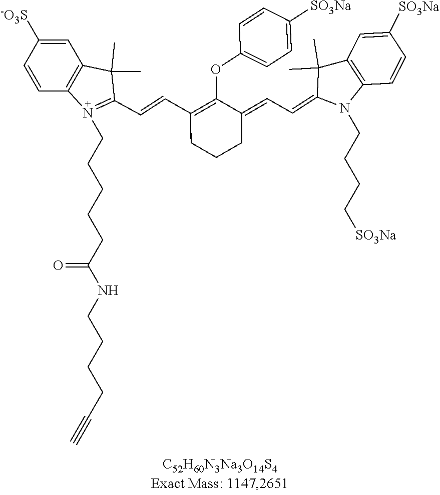

To a solution of IRDye®8000W Carboxylate (dye1, 10 mg, 9.36 μmol, 1.00 equiv.) in DMF (1.9 mL, 5 mM) were added DIPEA (3.2 μL, 18.7 μmol, 2.00 equiv.) and TSTU (5.6 mg, 18.7 μmol, 2.00 equiv.). The solution was stirred under light exclusion at rt for 30 min, until LCMS indicated complete conversion. The reaction mixture was inversely added to ice cold Et2O (2 mL) resulting in a green precipitate. After centrifugation the supernatant was discarded and the pellet was dissolved in PBS (1.9 mL, 5 mM). Hexynamine (1 μL, 10.3 μmol, 1.10 equiv.) was added and the reaction mixture was stirred at rt for 30 min. The reaction mixture was directly purified by column chromatography (C18 gold, 1%-95% MeCN+0.05% TFA/H₂O+0.05% TFA, main separation step at 15%). Cyanine dye2 (11.0 mg, 9.36 μmol, 100%) was obtained a's a green solid.

LC-MS (05-95_3.5 min): m/z 539 [M−3Na⁺+H⁺]²⁻, t_{R}=0.68 min; HRMS (ESI−): calculated for C₅₂H₆₀N₃O₁₄S₄³⁻ [M−3Na⁺]³⁻ 359.4319, observed 359.4319.

### 1.2.2 SSTR2-Targeting Peptides

### 1-azido-13-oxo-3,6,9-trioxa-12-azahexadecan-16-oic acid (linker2)

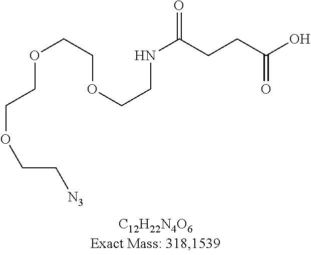 [SMILES: [N-]=[N+]=NCCOCCOCCOCCNC(=O)CCC(=O)O]

2-(2-(2-(2-azidoethoxy)ethoxy)ethoxy)ethan-1-amine (300 mg, 1.375 mmol, 1.00 equiv.) and dihydrofuran-2,5-dione (151 mg, 1.513 mmol, 1.10 equiv.) were dissolved in CH₂Cl₂ (3.8 mL, 0.4 M) and heated to reflux for 1.5 h. The reaction mixture was cooled to rt, concentrated under reduced pressure and the crude was purified by column chromatography (silica, 0%-20% MeOH/CH2Cl2, main separation step at 10%). Azide linker2 (320 mg, 1.01 mmol, 73%) was obtained a's a colorless oil.

R_{f} (10% MeOH/CH₂Cl₂)=0.2; ¹H NMR (400 MHz, CDCl₃): δ=6.81 (t, J=5.5 Hz, 1H), 3.56-3.47 (m, 10H), 3.42 (dd, J=5.7, 4.7 Hz, 2H), 3.32-3.24 (m, 4H), 2.52 (t, J=7.0 Hz, 2H), 2.39 (t, J=6.6 Hz, 2H) ppm; ¹³C NMR (101 MHz, CDCl₃): δ=175.2, 172.4, 70.2, 70.2, 70.1, 69.8, 69.7, 69.3, 50.3, 39.2, 30.4, 29.4 ppm; LC-MS (05-95_3.5 min): m/z 317 [M−H⁺]⁻, t_{R}=0.8 min; HRMS (ESI−): calculated for C₁₂H₂₁N₄O₆⁻ [M−H⁺]⁻ 317.1416, observed 317.1456.

### (9H-Fluoren-9-yl)methyl((2R,3R)-3-(tert-butoxy)-1-hydroxybutan-2-yl)carbamate (toc1)

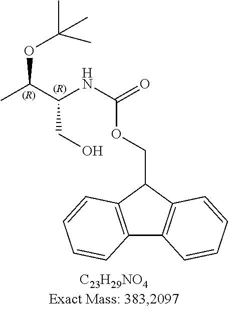

A solution of Fmoc-Thr(tBu)-OH (1.39 g, 3.5 mmol. 1.00 equiv.) in dry THF (30 mL, 0.12 M) was cooled to −10° C. N-Methylmorpholine (389 μL, 3.5 mmol, 1.00 equiv.) and ethyl chloroformate (333 μL, 3.5 mmol, 1.00 equiv.) were added successively. The reaction mixture was stirred at −10° C. for 30 min, followed by the addition of NaBH₄ (398 mg, 10.5 mmol, 3.00 equiv.). Over a period of 30 min, MeOH (60 mL) was slowly added to the reaction mixture, which was then stirred at 0° C. for 3 h. The reaction was ended by the addition of 1 M HCl (90 mL). The volatiles were removed at reduced pressure and the aqueous solution was extracted with CH2Cl2 (3×60 mL). The combined organic layers were dried over Na2SO4, filtered, concentrated under reduced pressure, and the crude was purified by column chromatography (C₁₈, 5%-95% MeCN/H₂O, main separation step at 60%). Fmoc-Thr(tBu)-ol (toc1, 1,2 g, 3.13 mmol, 90%) was obtained a's a white solid. The analytical data are in agreement with those reported in the literature (18).

R_{f} (Ethyl acetate)=0.73; ¹H NMR (400 MHz, CDCl₃): δ=7.77 (d, J=7.5 Hz, 2H), 7.61 (d, J=7.4 Hz, 2H), 7.41 (t, J=7.3 Hz, 2H), 7.3 (t, J=7.3 Hz, 2H), 5.27 (m, J=8.2 Hz, 1H), 4.48-4.37 (m, 2H), 4.24 (t, J=6.8 Hz, 1H), 3.99-3.92 (m, 1H), 3.76-3.67 (m, 2H), 3.67-3.57 (m, 1H), 1.22 (s, 9H), 1.18 (d, J=6.3 Hz, 3H) ppm; ¹³C NMR (101 MHz, CDCl₃): δ=157.2, 144.1, 141.5, 127.8, 127.2, 125.2, 120.1, 67.5, 67.0, 64.1, 57.2, 47.4, 28.8, 20.2 ppm; LC-MS (05-95_3.5 min): m/z 406 [M+Na⁺]⁺, t_{R}=1.34 min; HRMS (ESI+): calculated for C₂₃H₂₉NO₄Na⁺ [M+Na⁺]⁺ 406.1994, observed 406.1990.

### (9H-fluoren-9-yl)methyl tert-butyl (6-hydroxyhexane-1,5-diyl)(S)-dicarbamate (toc2)

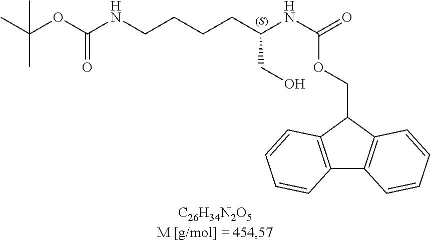

A solution of Fmoc-Lys(Boc)-OH (3.00 g, 6.4 mmol. 1.00 equiv.) in dry THF (32 mL, 0.2 M) was cooled to -10° C. DIPEA (1.11 mL, 6.5 mmol, 1.02 equiv.) and isobutyl chloroformate (831 μL, 6.4 mmol, 1.00 equiv.) were added successively. The reaction mixture was stirred at −10° C. for 30 min and the resulting precipitate was filtered and washed with THF (2×10 mL). The filtrate was cooled to −10° C. After addition of NaBH₄ (484 mg, 12.8 mmol, 2.00 equiv.) the solution was warmed to rt and stirred for 30 min. The volatiles were removed at reduced pressure and Ethyl acetate (100 mL) was added. The organic layer was washed with 1 M HCl (2×50 mL), saturated aqueous NaHCO₃ (2×50 mL) and brine (2×50 mL), concentrated under reduced pressure, and the crude was purified by column chromatography (C18, 30%-95% MeCN/H₂O, main separation step at 60%). %). Fmoc-Lys(Boc)-ol (toc2, 1.50 g, 3.3 mmol, 52%) was obtained a's a white solid. The analytical data are in agreement with those reported in the literature (19).

R_{f} (10% MeOH/CH₂Cl₂)=0.56; ¹H NMR (400 MHz, CDCl₃): δ=7.76 (d, J=7.6 Hz, 2H), 7.60 (d, J=7.5 Hz, 2H), 7.40 (t, J=7.5 Hz, 2H), 7.32 (td, J=7.5, 1.2 Hz, 2H), 5.05 (bs, 1H), 4.58 (bs, 1H), 4.41 (bs, 2H), 4.21 (t, J=6.8 Hz, 1H), 3.65 (d, J=12.8 Hz, 3H), 3.25-3.02 (m, 2H), 1.54-1.31 (m, 15H) ppm; ¹³C NMR (101 MHz, CDCl₃): δ=156.8, 156.5, 144.0, 141.5, 127.8, 127.2, 125.2, 120.1, 79.5, 66.7, 64.8, 53.1, 47.4, 39.7, 30.5, 30.1, 28.54, 22.7 ppm; LC-MS (05-95_3.5 min): m/z 477 [M+Na⁺]⁺, t_{R}=1.31 min; HRMS (ESI+): calculated for C₂₆H₃₄N₂O₅Na⁺ [M+Na⁺]⁺ 477.2365, observed 477.2359.

### (1-azido-13-oxo-3,6,9-trioxa-12-azahexadecan-16-oyl)-phe-cyclo(Cys-Tyr-trp-Lys-Thr-Cys)-Thr(ol) (toc4)

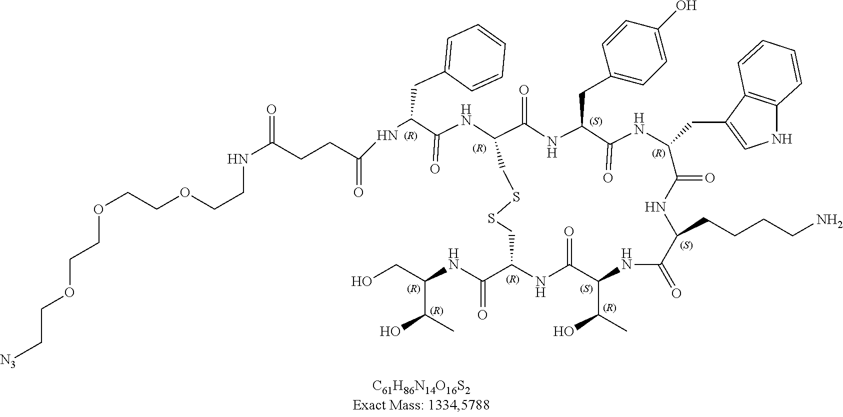

Automated peptide synthesis at 100 μmol scale with preloaded DHP-HM resin, cleavage and precipitation was performed a's described in 1.1 and afforded linear peptide toc3. The crude peptide was purified by column chromatography (C18 gold, 1%-95% MeCN+0.05% TFA/H₂O+0.05% TFA, main separation step at 40%). For disulfide cyclization, linear peptide toc3 (40 mg) was dissolved in 40 mL of THF/5 mM ammonium acetate (4/1). The solution was buffered to pH=8 with aqueous NaHCO₃ and 4 μL 30% H₂O₂ were added. The reaction mixture was stirred 1 h, THF was removed at reduced pressure and the solution was lyophilized. The residue was purified by column chromatography (C18 gold, 1%-95% MeCN+0.05% TFA/H₂O+0.05% TFA, main separation step at 40%). Cyclized peptide toc4 (X) was obtained a's a white solid. Low yield was accepted in favor of purity.

LC-MS (01-95_5 min): m/z 1335 [M+H⁺]⁺, t_{R}=2.62 min, HRMS (ESI+): calculated for C₆₁H₈₇N₁₄O₁₆S₂⁺ [M+H⁺]⁺ 1335.5866, observed 1335.5880.

### (1-azido-13-oxo-3,6,9-trioxa-12-azahexadecan-16-oyl)-trp-cyclo(Cys-Thr-phe-Thr-Tyr-Cys)-Lys(ol) (toc6)

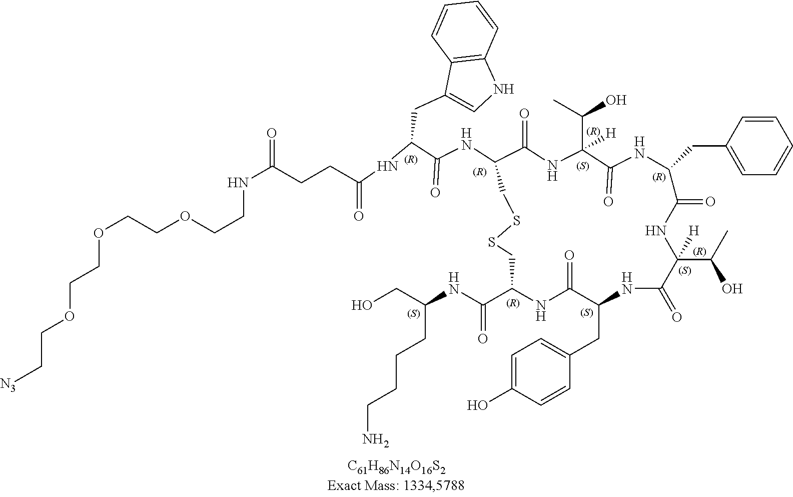

Automated peptide synthesis at 100 μmol scale with preloaded DHP-HM resin, cleavage and precipitation was performed a's described in 1.1 and afforded linear peptide toc5. The crude peptide was purified by column chromatography (C18 gold, 1%-95% MeCN+0.05% TFA/H₂O+0.05% TFA, main separation step at 40%). For disulfide cyclization, linear peptide toc5 (40 mg) was dissolved in 40 mL of THF/5 mM ammonium acetate (4/1). The solution was buffered to pH=8 with aqueous NaHCO₃ and 4 μL 30% H₂O₂ were added. The reaction mixture was stirred 1 h, THF was removed at reduced pressure and the solution was lyophilized. The residue was purified by column chromatography (C18 gold, 1%-95% MeCN+0.05% TFA/H₂O+0.05% TFA, main separation step at 40%). Cyclized peptide toc6 (X) was obtained a's a white solid. Low yield was accepted in favor of purity.

LC-MS (01-95_5 min): m/z 1335 [M+H⁺]⁺, t_{R}=2.62 min, HRMS (ESI+): calculated for C₆₁H₈₆N₁₄NaO₁₆S₂⁺ [M+Na⁺]⁺ 1357.5685, observed 1357.5688.

### (1-azido-13-oxo-3,6,9-trioxa-12-azahexadecan-16-oyl)-phe-cyclo(Cys-Tyr-trp-Lys-Thr-Cys)-Thr-OH (tate2)

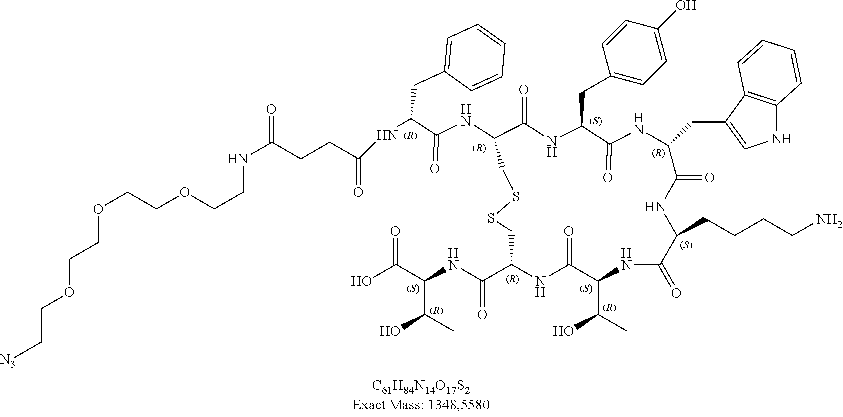

Automated peptide synthesis at 100 μmol scale with preloaded 2-chlorotrityl chloride resin, cleavage and precipitation was performed a's described in 1.1 and afforded linear peptide tate1. The crude peptide was purified by column chromatography (C18 gold, 1%-95% MeCN+0.05% TFA/H₂O+0.05% TFA, main separation step at 35%). For disulfide cyclization, linear peptide tate1 (40 mg) was dissolved in 40 mL of THF/5 mM ammonium acetate (4/1). The solution was buffered to pH=8 with aqueous NaHCO₃ and 4 μL 30% H₂O₂ were added. The reaction mixture was stirred 1 h, THF was removed at reduced pressure and the solution was lyophilized. The residue was purified by column chromatography (C18 gold, 1%-95% MeCN+0.05% TFA/H₂O+0.05% TFA, main separation step at 35%). Cyclized peptide tate2 (X) was obtained a's a white solid. Low yield was accepted in favor of purity.

LC-MS: m/z 1349 [M+H⁺]⁺, t_{R}=2.65 min, HRMS (ESI−): calculated for C₆₁H₈₃N₁₄O₁₇S₂⁻ [M−H⁺]⁻ 1347.5502, observed 1347.5497.

### (1-azido-13-oxo-3,6,9-trioxa-12-azahexadecan-16-oyl)-trp-cyclo(Cys-Thr-phe-Thr-Tyr-Cys)-Lys-OH (tate4)

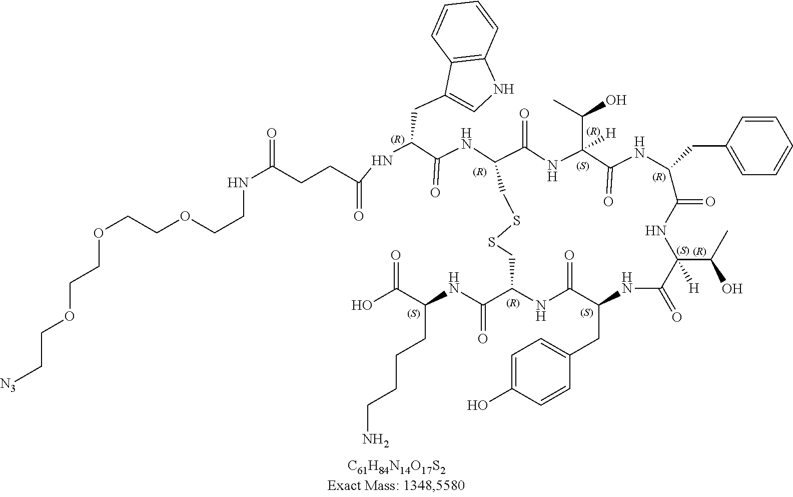

Automated peptide synthesis at 100 μmol scale with preloaded 2-chlorotrityl chloride resin, cleavage and precipitation was performed a's described in 1.1 and afforded linear peptide tate3. The crude peptide was purified by column chromatography (C18 gold, 1%-95% MeCN+0.05% TFA/H₂O+0.05% TFA, main separation step at 35%). For disulfide cyclization, linear peptide tate3 (40 mg) was dissolved in 40 mL of THF/5 mM ammonium acetate (4/1). The solution was buffered to pH=8 with aqueous NaHCO₃ and 4 μL 30% H₂O₂ were added. The reaction mixture was stirred 1 h, THF was removed at reduced pressure and the solution was lyophilized. The residue was purified by column chromatography (C18 gold, 1%-95% MeCN+0.05% TFA/H₂O+0.05% TFA, main separation step at 35%). Cyclized peptide tate4 (X) was obtained a's a white solid. Low yield was accepted in favor of purity.

LC-MS: m/z 1349 [M+H⁺]⁺, t_{R}=2.65 min, HRMS (ESI−): calculated for C₆₁H₈₃N₁₄O₁₇S₂⁻ [M−H⁺]⁻ 1347.5502, observed 1347.5497.

### 1.2.3 SSTR2-Targeting NIR-Fluorescent Probes

sstr2click1

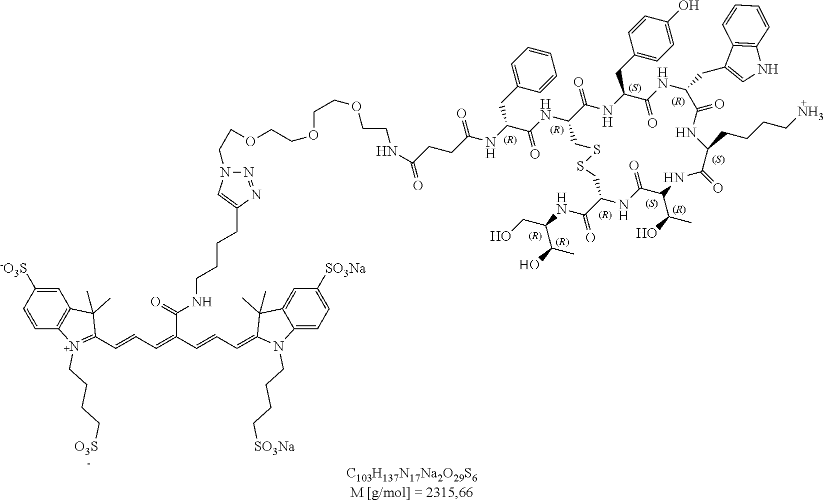

Cyclized peptide toc4 (2.00 mg, 1.50 μmol, 1.00 equiv.) and cyanine dyealkin5 (1.50 mg, 1.50 μmol, 1.00 equiv.) were under light exclusion dissolved in degassed MeOH (250 μL, 6 mM). In parallel, THPTA (2.60 mg, 5.99 μmol, 4.00 equiv.) and sodium ascorbate (0.89 mg, 4.50 μmol, 4.00 equiv.) were added to degassed aqueous CuSO₄ (50 mM, 120 μL, 5.99 μmol, 4.00 equiv.). The copper (I) containing solution was pipetted to the solution of azide and alkyne. The reaction mixture was flushed with argon, sonicated for 1 min, stirred at rt for 2 h and then directly purified by column chromatography (C18 gold, 5%-95% MeCN+0.05% TFA/H₂O+0.05% TFA, main separation step at 25%). Probe sstr2click1 (1.40 mg, 0.61 μmol, 40%) was obtained a's a green solid.

LC-MS (05-95_13 min): m/z 1136 [M−2Na⁺+4H⁺]²⁺, t_{R}=6.33 min; HRMS (ESI−): calculated for C₁₀₃H₁₃₆N₁₇O₂₉S₂³⁻ [M−2Na⁺−H]³⁻ 755.6005, observed 755.6006.

sstr2click2

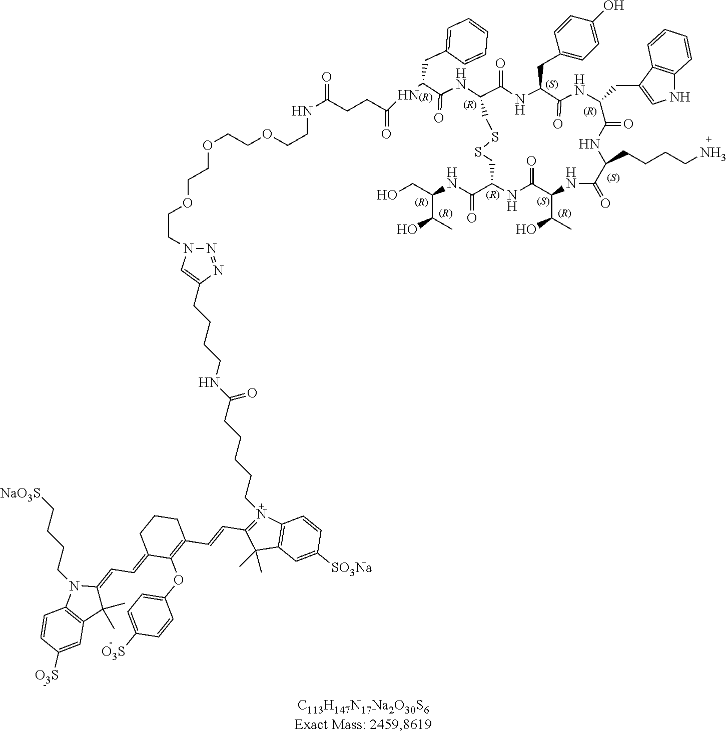

Cyclized peptide toc4 (1.50 mg, 1.12 μmol, 1.00 equiv.) and cyanine dye2 (1.23 mg, 1.50 μmol, 0.95 equiv.) were under light exclusion dissolved in degassed tert-BuOH (180 μL, 6 mM). In parallel, THPTA (1.95 mg, 4.50 μmol, 4.00 equiv.) and sodium ascorbate (0.89 mg, 4.50 μmol, 4.00 equiv.) were added to degassed aqueous CuSO₄ (50 mM, 45 μL, 2.25 μmol, 2.00 equiv.). The copper (I) containing solution was pipetted to the solution of azide and alkyne. The reaction mixture was flushed with argon, sonicated for 1 min, stirred at rt for 2 h and then directly purified by column chromatography (C18 gold, 5%-95% MeCN+0.05% TFA/H₂O+0.05% TFA, main separation step at 25%). Probe sstr2click2 (1.26 mg, 0.52 μmol, 49%) was obtained a's a green solid.

LC-MS (05-95_13 min): m/z 1209 [M−2Na⁺+4H⁺]²⁺, t_{R}=6.62 min; HRMS (ESI−): calculated for C₁₁₃H₁₄₆N₁₇O₃₀S₆³⁻ [M−2Na⁺−H]³⁻ 805.2977, observed 805.6284.

sstr2click3

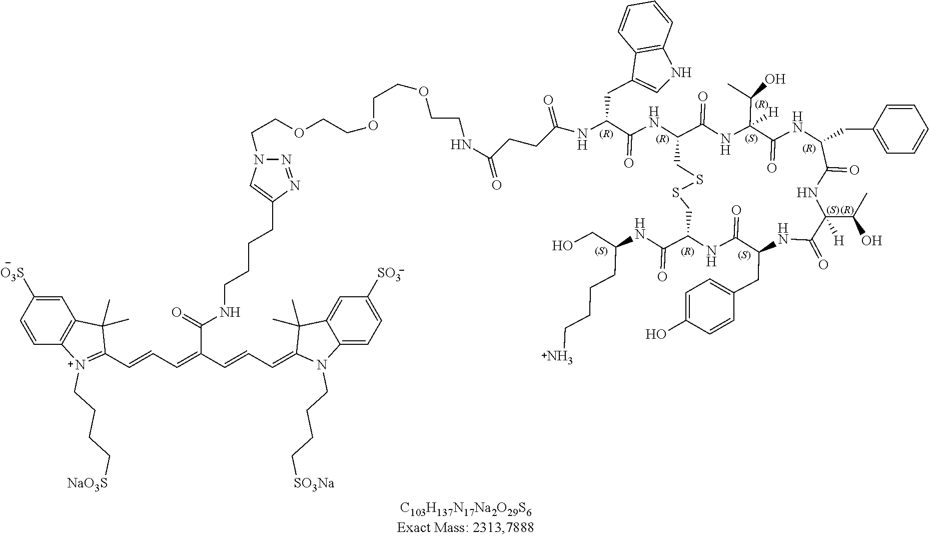

Cyclized peptide toc6 (3.30 mg, 2.47 μmol, 1.00 equiv.) and cyanine dyealkin5 (2.23 mg, 2.22 μmol, 0.90 equiv.) were under light exclusion dissolved in degassed water (370 μL, 6 mM). In parallel, THPTA (4.29 mg, 9.88 μmol, 4.00 equiv.) and sodium ascorbate (1.47 mg, 7.41 μmol, 3.00 equiv.) were added to degassed aqueous CuSO₄ (50 mM, 198 μL, 9.88 μmol, 4.00 equiv.). The copper (I) containing solution was pipetted to the solution of azide and alkyne. The reaction mixture was flushed with argon, sonicated for 1 min, stirred at rt for 2 h and then directly purified by column chromatography (C18 gold, 5%-95% MeCN+0.05% TFA/H₂O+0.05% TFA, main separation step at 25%). Probe sstr2click3 (2.30 mg, 1.01 μmol, 46%) was obtained a's a green solid.

LC-MS (05-95_13 min): m/z 1136 [M−2Na⁺+4H⁺]²⁺ t_{R}=6.56 min, HRMS (ESI−): calculated for C₁₀₃H₁₃₆N₁₇O₂₉S₂³⁻ [M−2Na⁺−H⁺]³⁻ 755.6005, observed 755.6006.

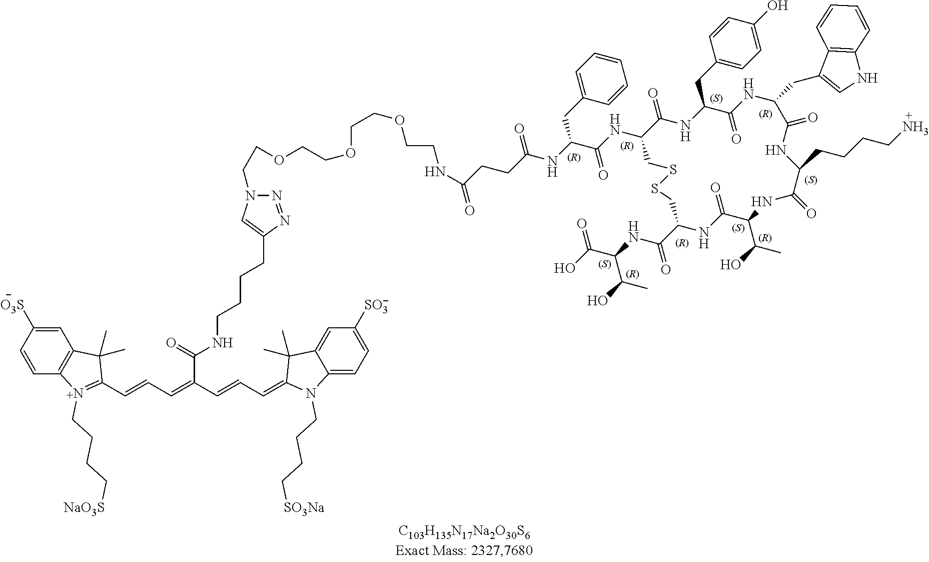

sstr2click4
Cyclized peptide tate2 (5.00 mg, 3.71 μmol, 1.00 equiv.) and cyanine dyealkin5 (3.53 mg, 3.52 μmol, 0.95 equiv.) were under light exclusion dissolved in degassed water (590 μL, 6 mM). In parallel, THPTA (6.44 mg, 14.8 μmol, 4.00 equiv.) and sodium ascorbate (2.20 mg, 11.1 μmol, 3.00 equiv.) were added to degassed aqueous CuSO₄ (50 mM, 297 μL, 14.8 μmol, 4.00 equiv.). The copper (I) containing solution was pipetted to the solution of azide and alkyne. The reaction mixture was flushed with argon, sonicated for 1 min, stirred at rt for 2 h and then directly purified by column chromatography (C18 gold, 5%-95% MeCN+0.05% TFA/H₂O+0.05% TFA, main separation step at 25%). Probe sstr2click4 (1.98 mg, 0.81 μmol, 23%) was obtained a's a green solid.

LC-MS (05-95_13 min): m/z 1143 [M−2Na⁺+4H⁺]²⁺ t_{R}=7.62 min, HRMS (ESI−): calculated for C₁₀₃H₁₃₄N₁₇O₃₀S₂³⁻ [M−2Na⁺−H⁺]³⁻ 760.2602, observed 760.2604.

sstr2click5

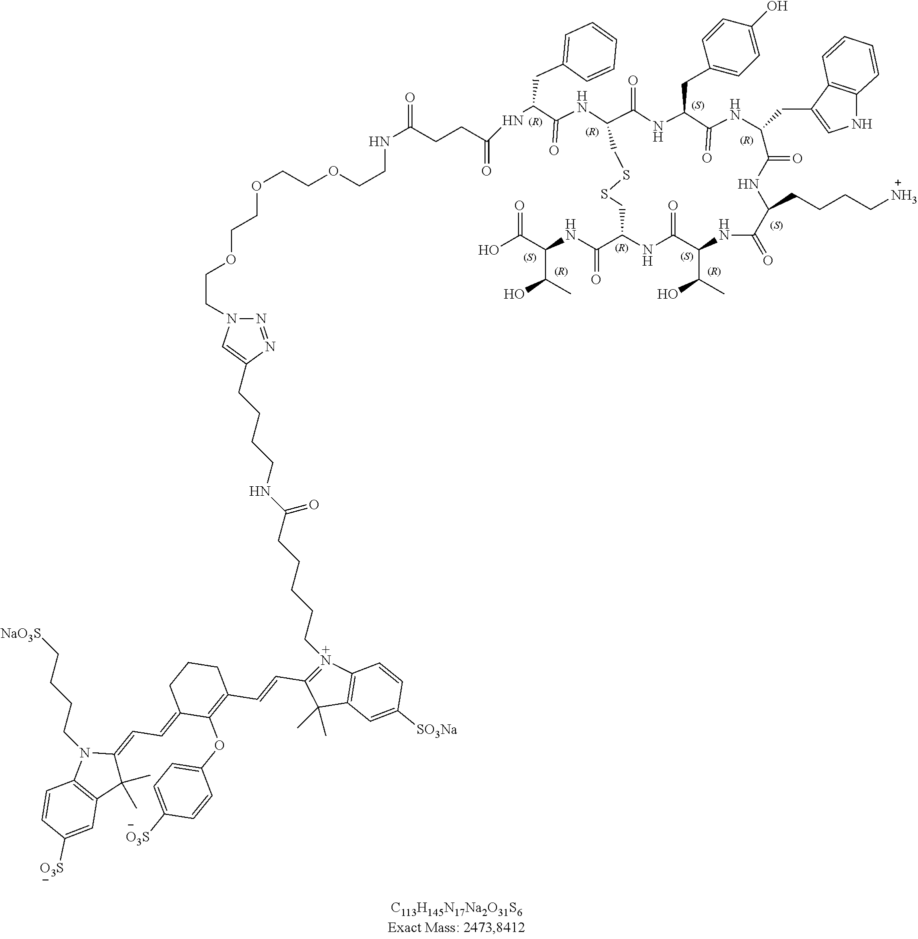

Probe sstr2click5 was synthesized in a similar fashion a's described for sstr2click3.

LC-MS (05-95_13 min): m/z 1216 [M−2Na⁺+4H⁺]²⁺ t_{R}=7.68 min.

sstr2click6

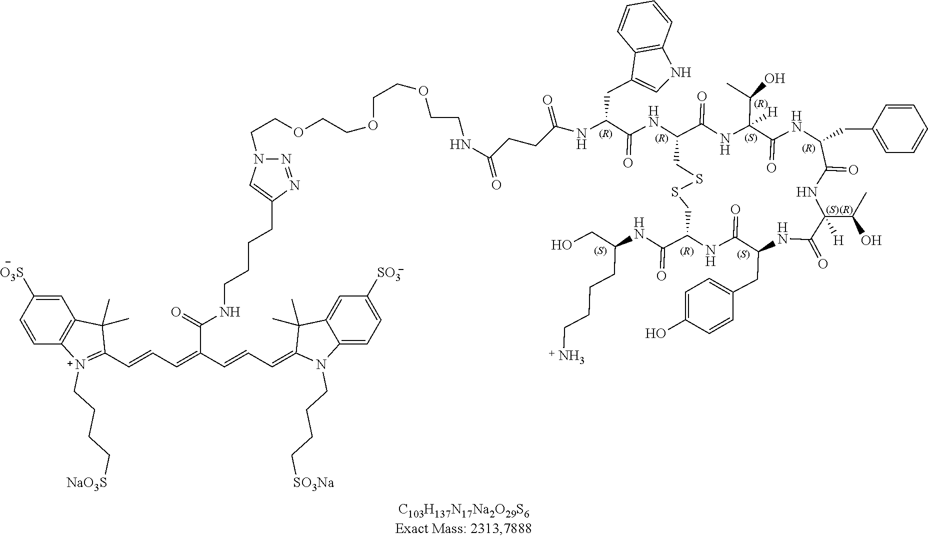

Probe sstr2click6 was synthesized in a similar fashion a's described for sstr2click2.

LC-MS (05-95_13 min): m/z 1143 [M−2Na⁺+4H⁺]²⁺ t_{R}=8.63 min, HRMS (ESI−): calculated for C₁₀₃H₁₃₅N₁₇O₃₀S₂²⁻ [M−2Na⁺]²⁻ 1140.8943, observed 1140.8931.

### PSMA0

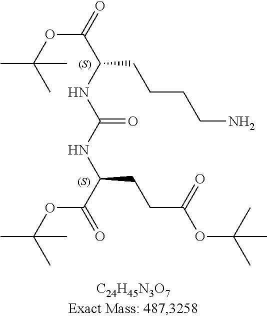

Urea PSMA0 was synthesized according to literature (20, compound 3).

### PSMA1

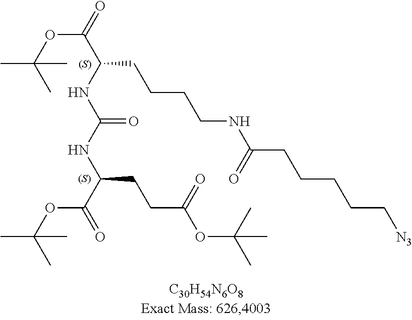

Amine PSMA0 (190 mg, 0.39 mmol, 1.0 equiv.) and N3-(CH₂)₅—CO—OSu (100 mg, 0.39 mmol, 1.0 equiv.) were dissolved in CHCl₃ (2 mL, 0.1 M), stirred at rt for 3 h and concentrated under reduced pressure. After purification by column chromatography (C18 gold, H₂O/MeCN, 5%-95%, main separation step at 80%), urea PSMA1 (223 mg, 0.36 mmol, 91%) was obtained a's a colorless oil.

LC-MS (01-90_5 min): m/z 628 [M+H⁺]⁺, t_{R}=3.80 min; HRMS (ESI+): calculated for C₃₀H₅₄N₆O₈Na⁺ [M+Na⁺]⁺ 810.4589, observed 810.4564; ¹H NMR (400 MHz, CDCl₃): δ=4.34-4.24 (m, 2H), 3.27 (t, J=6.5 Hz, 3H), 2.39-2.26 (m, 2H), 2.20 (t, J=7.7 Hz, 2H), 2.12-2.03 (m, 1H), 1.90-1.73 (m, 2H), 1.69-1.58 (m, 5H), 1.54-1.36 (m, 33H) ppm; ¹³C NMR (400 MHz, CDCl₃): δ=173.2, 172.5, 157.2, 128.6, 82.4, 81.9, 81.9, 80.8, 53.6, 53.3, 51.5, 39.1, 36.5, 32.7, 31.8, 28.9, 28.8, 28.3, 28.8, 28.8, 26.5, 25.7, 25.4, 24.3 ppm.

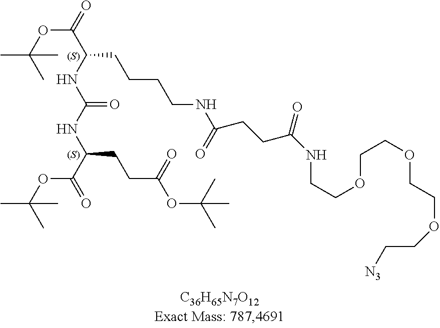

Amine PSMA0 (100 mg, 0.24 mmol, 1.0 equiv.) and N3-PEG(3)-NHCO—(CH₂)₂—CO—OSu (117 mg, 0.24 mmol, 1.0 equiv.) were dissolved in CHCl₃ (2 mL, 0.1 M), stirred at rt for 3 h and concentrated under reduced opressure. After purification by column chromatography (C18 gold, H₂O/MeCN, 5%-95%, main separation step at 75%), urea PSMA2 (91 mg, 0.12 mmol, 48%) was obtained a's a colorless oil.

LC-MS (01-90_5 min): m/z 798 [M+H⁺]⁺, t_{R}=3.64 min; HRMS (ESI+): calculated for C₃₆H₆₅N₇O₁₂Na⁺ [M+Na⁺]⁺ 810.4589, observed 810.4564; ¹H NMR (400 MHz, CDCl₃): δ=7.03 (m, 2H), 6.83 (m, 2H), 6.08 (d, J=8.6 Hz, 2H), 5.86 (d, J=8.9 Hz, 2H), 5.45 (m, 7H), 4.33-4.25 (m, 13H), 3.65-3.56 (m, 25H), 3.52-3.43 (m, 8H), 3.37-3.34 (m, 10H), 2.69-2.59 (m, 11H), 4.33-4.25 (m, 13H), 2.43-2.21 (m, 19H), 2.07-1.97 (m, 12H), 1.83-1.66 (m, 12H) ppm; ¹³C NMR (400 MHz, CDCl₃): δ=173.5, 172.8, 172.4, 172.4, 157.6, 157.1, 82.0, 81.6, 81.1, 80.5, 70.7, 70.3, 70.1, 69.8, 53.5, 53.0, 50.7, 41.8, 39.4, 38.7, 33.0, 31.8, 31.7, 31.5, 31.2, 28.1, 28.1, 22.5, 21.6 ppm.

### PSMA3

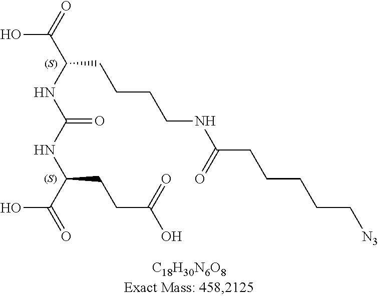

Urea PSMA1 (100 mg, 0.16 mmol, 1.0 equiv.) was dissolved in CH₂Cl₂/TFA (1:1, 4 mL, 0.02 M), stirred over night at rt and concentrated under reduced pressure. After column chromatographic purification (C18 gold, H₂O+0.05% TFA/MeCN+0.05% TFA, 5%-95%, main separation step at 42%) urea PSMA3 (18 mg, 0.04 mmol, 24%) was obtained a's a yellow oil.

LC-MS (01-90_5 min): m/z 459 [M+H⁺]⁺, t_{R}=2.39 min; HRMS (ESI+): calculated for C₁₈H₃₀N₆O₈Na⁺ [M+Na⁺]⁺ 481.2023, observed 481.2018; ¹H NMR (400 MHz, MeOD): δ=4.33 (q, J=4.9 Hz, 1H), 4.28 (q, J=4.9 Hz, 1H), 3.37 (s, 1H), 3.34-3.29 (m, 1H), 3.19 (t, J=6.9 Hz, 2H), 2.46-2.41 (m, 2H), 2.23-2.12 (m, 3H), 1.96-1.82 (m, 2H), 1.73-1.61 (m, 5H), 1.59-1.51 (m, 2H), 1.48-1.39 (m, 4H) ppm; ¹³C NMR (400 MHz, MeOD): δ=176.4, 176.4, 176.0, 175.8, 160.1, 53.9, 53.5, 52.3, 40.1, 36.9, 33.2, 31.1, 29.9, 29.6, 28.9, 27.4, 26.5, 24.0 ppm.

### PSMA4

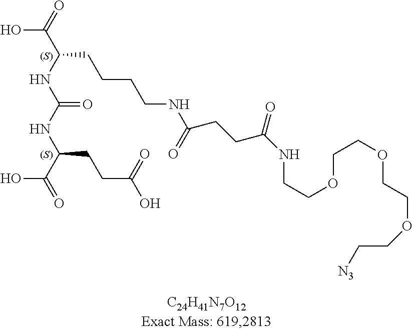

Urea PSMA2 (100 mg, 0.13 mmol, 1.0 equiv.) was dissolved in CH₂Cl₂/TFA (1:1, 4 mL, 0.02 M), stirred over night at rt and concentrated under reduced pressure. After column chromatographic purification (C18 gold, H₂O+0.05% TFA/MeCN+0.05% TFA, 5%-95%, main separation step at 30%), urea PSMA4 (23 mg, 0.04 mmol, 29%) was obtained a's a yellow oil.

LC-MS (01-90_5 min): m/z 620 [M+H⁺]⁺, t_{R}=2.32 min; HRMS (ESI−): calculated for C₂₄H₄₀N₇O₁₂⁺ [M−H⁺]⁻ 618.2735, observed 618.2725; ¹H NMR (400 MHz, MeOD): δ=4.31 (q, J=5.3 Hz, 1H), 4.26 (q, J=4.8 Hz, 1H), 3.68-3.60 (m, 8H), 3.55-3.53 (m, 2H), 3.39-3.35 (m, 3H), 3.23-3.10 (m, 2H), 2.50-2.39 (m, 5H), 2.19-2.10 (m, 1H), 1.94-1.79 (m, 2H), 1.70-1.61 (m, 1H), 1.57-1.39 (m, 4H) ppm; ¹³C NMR (400 MHz, MeOD): δ=176.4, 176.4, 175.9, 174.8, 174.6, 160.1, 71.6, 71.6, 71.5, 71.3, 71.1, 70.5, 53.9, 53.5, 51.8, 40.4, 40.0, 33.1, 32.3, 32.3, 31.1, 29.8, 28.9, 23.8 ppm.

### NIR-Fluorescent KUE

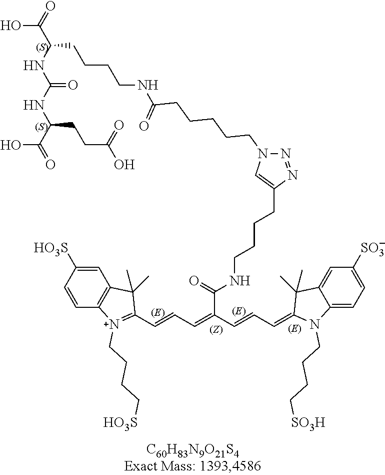

A solution of urea PSMA3 (5.00 mg, 0.01 mmol, 1.0 equiv.) in tBuOH (1 mL, 0.01 M) was added to a solution of dyealkin5 (10.0 mg, 0.01 mmol, 0.95 equiv.) in water (1 mL, 0.01 M). The mixture was combined with an aqueous solution of CuSO₄ (0.05 M, 872 μL, 0.04 mmol, 4.0 equiv.), THPTA (19.0 mg, 0.04 mmol, 4.0 equiv.) and sodium ascorbate (18.0 mg, 0.09 mmol, 8.0 equiv.) and stirred for 2 h at rt. The reaction solution was directly purified by column chromatography (C18 gold, H₂O+0.05% TFA/MeCN+0.05% TFA, 5%-90%, main separation step at 20%), NIR-fluorescent KUE Ligand I (4.2 mg, 3.01 μmol, 29%) was obtained a's a green solid.

LC-MS (05-95_5 min): m/z 696 [M−2H]²⁻, t_{R}=5.86 min.

### NIR-Fluorescent KUE

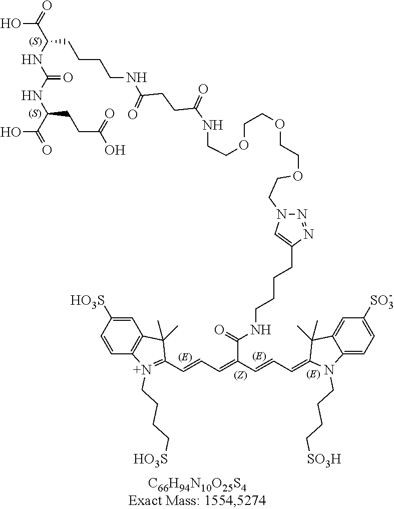

A solution of urea PSMA4 (5.00 mg, 0.01 mmol, 1.0 equiv.) in tBuOH (1 mL, 0.01 M) was added to a solution of dyealkin5 (8.0 mg, 0.01 mmol, 0.95 equiv.) in water (1 mL, 0.01 M). The mixture was combined with an aqueous solution of CuSO₄ (0.05 M, 872 μL, 0.04 mmol, 4.0 equiv.), THPTA (19.0 mg, 0.04 mmol, 4.0 equiv.) and sodium ascorbate (18.0 mg, 0.09 mmol, 8.0 equiv.) and stirred for 2 h at rt. The reaction solution was directly purified by column chromatography (C18 gold, H₂O+0.05% TFA/MeCN+0.05% TFA, 5%-90%, main separation step at 20%), NIR-fluorescent KUE Ligand II (2.7 mg, 1.7 μmol, 17%) was obtained a's a green solid.

LC-MS (05-95_5 min): m/z 777 [M−2H]²⁻, t_{R}=5.93 min.

### NIR-Fluorescent Panitumumab

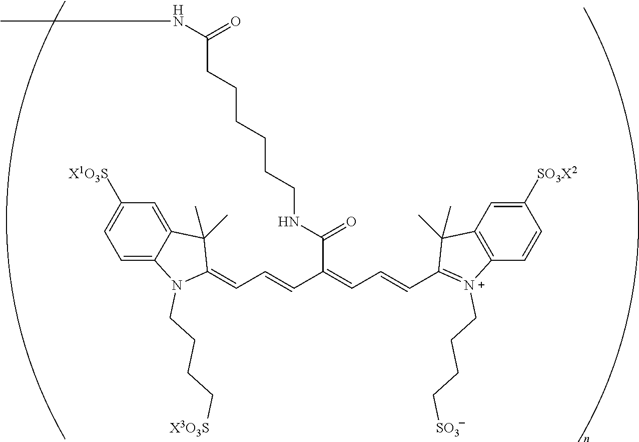

Panitumumab (20 mg/mL commercial stock solution, 50 μL, 7 nmol) was added to 25 μL of PBS (50 mM, pH 7.4) in a 1.5 mL microcentrifuge tube. Dyecarb7 (1 mM stock in DMSO, 50 μL, 7 equiv) was added to the Panitumumab solution, vortexed and incubated at room temperature in the dark for 100 min. The resulting solutions were eluted through a Zeba spin desalting column (7 kDa MWCO, Thermo Fisher Scientific) to remove unreacted free dye. UVNIS of the filtrate dilution was acquired directly and used to calculate a density of labeling (DOL) of 1.6 using the following equation:

$$DOL = \frac{{A}_{785}}{{ε}_{sNIR 785}} \div \frac{{A}_{280} - 0.064 * {A}_{785}}{{ε}_{Protein}}$$

Antibody fluorophore conjugates were used for animal studies within 1 week after labeling. Panitumumab-IRDye800 was prepared in a similar fashion.

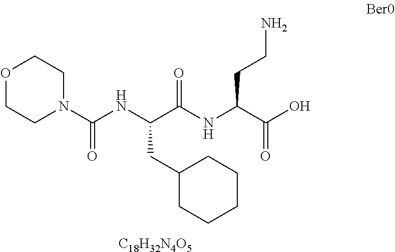 [SMILES: NCC[C@H](NC(=O)[C@H](CC1CCCCC1)NC(=O)N1CCOCC1)C(=O)O ; "C18H32N4O5"]

Peptide ber0 was synthesized according to literature (21, compound 3).

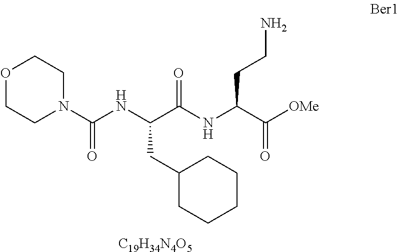 [SMILES: COC(=O)[C@H](CCN)NC(=O)[C@H](CC1CCCCC1)NC(=O)N1CCOCC1 ; "C19H34N4O5"]

A solution of peptide ber0 (130 mg, 0.27 mmol, 1.0 equiv.) in MeOH (270 μL, 1.0 M) was cooled to 0° C. and SOCl₂ (60 μL, 0.81 mmol, 3.0 equiv.) was added. The reaction was stirred for 2 h at room temperature. The solvent was removed in vacuo to afford the desired product ber1 (95 mg, 0.21 mmol, 78%) a's a colorless solid.

LCMS (Agilent, unpol, C8): t_{R}=0.13, m/z=421 [M+Na]⁺; ESI-HRMS: m/z calc. for C₁₉H₃₄N₄O₅Na [M+Na]⁺: 421,2427 observed: 421.2427; ¹H-NMR (400 MHz, MeOD) δ=4.66-4.54 (m, 1H, CH), 4.30-4.14 (m, 1H, CH), 3.76 (s, 3H, CH₃), 3.70-3.61 (m, 4H, H-1,1′), 3.48-3.52 (m, 4H, H-2,2′), 3.07-3.01 (m, 2H, H-13), 2.33-2.21 (m, 1H, H-12), 2.10-1.97 (m, 1H, H-12), 1.81-1.19 (m, 11H, H-5,6,7,8,9,10,4), 1.03-0.92 (m, 2H, H-5,6,7,8,9,10,4) ppm; ¹³C-NMR (100 MHz, MeOD) δ=177.2 (C_{q}), 172.5 (C_{q}), 159.9 (C_{q}), 67.6 (C-1,1′), 54.4 (CH), 53.1 (CH₃), 50.9 (CH), 45.3 (C-2,2′), 40.2 (C-13), 37.7 (C-4), 35.3 (C-6,7,8,9,10), 34.7 (C-6,7,8,9,10), 33.7 (C-12), 30.6 (C-5), 27.6 (C-6,7,8,9,10), 27.4 (C-6,7,8,9,10), 27.3 (C-6,7,8,9,10) ppm.

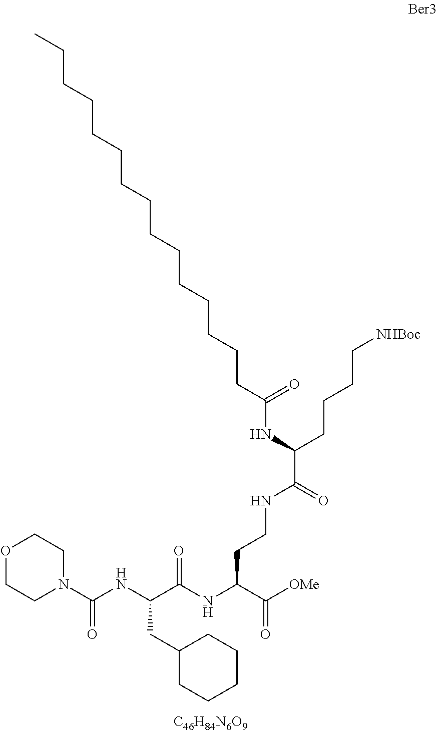

A solution of peptide ber1 (50 mg, 0.125 mmol, 1.0 equiv.) was dissolved in DMF (250 μL, 0.02 M) and mixed with side chain ber2 (synthesized a's described in literature (20, compound 8), 49 mg, 0.100 mmol, 0.8 equiv.), HATU (72 mg, 0.188 mmol, 1.5 equiv.) and DIPEA (110 μL, 0.627 mmol, 5.0 equiv.) and stirred for 20 h at room temperature. Column chromatographic purification (CH₂Cl₂/MeOH=0-10%) afforded the desired Boc-protected amine ber3 (76 mg, 0.003 mmol, 88%) a's a colorless solid.

LCMS (Thermofisher, PFP): t_{R}=10.09, m/z=866 [M+H]⁺; ESI-HRMS: m/z calc. für C₈₁H₁₁₇N₈O₂₀S₄⁺ [M−3K⁺4H]⁺: 1649.7267, gefunden: 1649.7327; ¹H-NMR (600 MHz, CDCl₃) b=6.27-6.03 (m, 5H, NH), 3.76-3.66 (m, 10H, CH, CH₂, O—CH₃), 3.43-3.33 (m, 1H; CH₂), 3.22-3.15 (m, 8H, CH, CH₂), 3.12-3.04 (m, 1H, CH₂), 3.01-2.95 (m, 1H, CH), 1.49-1.39 (m, 48H, CH, CH₂, CH₃), 1.30-1.22 (m, 9H, CH₂), 0.87 (t, 3H, J=7.0 Hz, CH₃) ppm; ¹³C-NMR (150 MHz, CDCl₃) δ=175.7 (C_{q}), 174.0 (C_{q}), 172.6 (C_{q}), 171.6 (C_{q}), 158.0 (C_{q}), 156.2 (C_{q}), 79.3 (C_{q}), 66.6 (CH₂), 55.9 (CH₃), 53.9 (CH), 52.6 (CH), 52.2 (CH), 43.8 (CH₂), 32.1 (CH₂), 29.8 (CH₃), 29.5 (CH₃), 28.6 (CH), 22.8 (CH₂), 18.7 (CH₂), 17.3 (CH₂), 12.7 (CH₃), ppm.

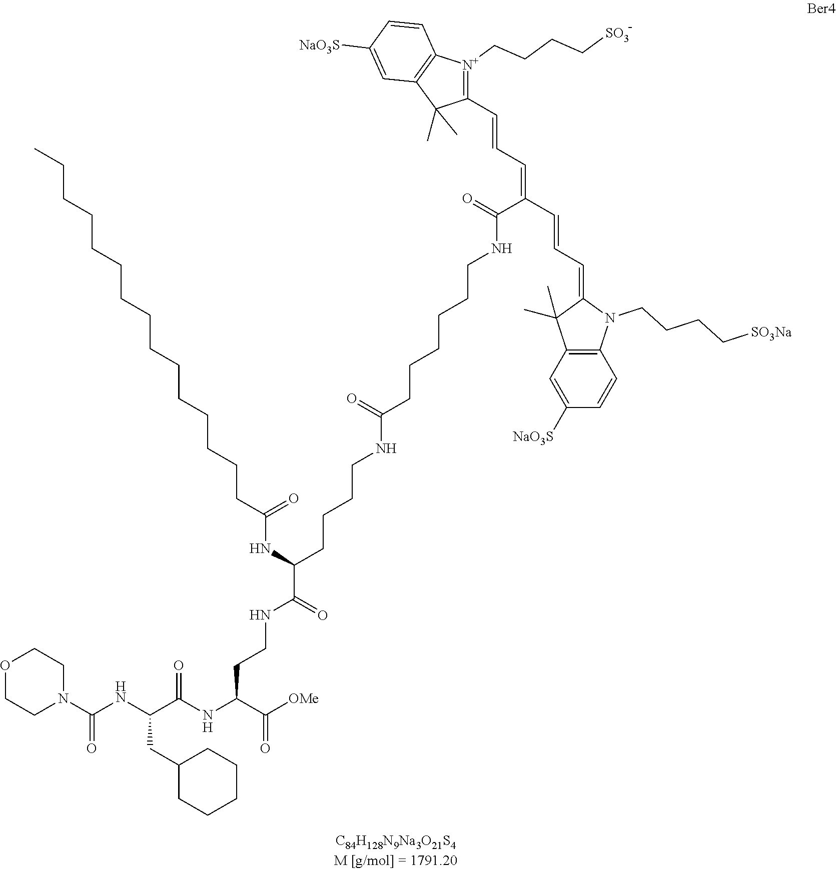

A solution of Boc-protected amine ber3 (3 mg, 0.004 mmol, 1.0 equiv.) in TFA/CH₂Cl₂ (400 μL, 0.01 M) was stirred at rt for 30 min and then concentrated under reduced pressure. The residue was dissolved in DMF (400 μL, 0.01 M) and mixed with dyecarb7 (3 mg, 0.004 mmol, 1.0 equiv.) and DIPEA (3 μL, 0.020 mmol, 5.0 equiv.) and stirred for 1 h at room temperature. Column chromatographic purification (C18 WP, H₂O(TFA)/ACN(TFA)=5-95%) afforded the desired methylester ber4 (2 mg, 0.002 mmol, 42%) a's a green solid.

LCMS (Thermofisher PFP): t_{R}=8.69, m/z=866 [M−3Na⁺5H]²⁺; ESI-HRMS: m/z calc. für C₈₄H₁₃₁N₉O₂₁S₄Na [M−2Na⁺3H]⁺: 1752.8240, gefunden: 1752.8240; ¹H-NMR (600 MHz, MeOD) δ=7.92-7.83 (m, 6H, CH_{aromatisch}), 7.34 (d, 2H, J=8.3 Hz, CH_{aromatisch}), 6.53 (dd, 4H, J=39.9, 13.4 Hz, CH_{aromatisch}), 4.46 (dd, 1H, J=10.5, 4.3 Hz, CH), 4.30 (dd, 1H, J=9.0, 6.3 Hz, CH), 4.21-4.14 (m, 5H, CH, CH₂), 3.71 (s, 3H, O—CH₃), 3.66-3.62 (m, 4H, CH₂), 3.51-3.47 (m, 2H, CH₂), 3.43-3.39 (m, 2H, CH₂), 3.26-3.22 (m, 2H, CH₂), 3.16 (t, 2H, J=7.1 Hz, CH₂), 2.90 (t, 4H, J=6.9 Hz, CH₂), 2.25 (t, 2H, J=7.5 Hz, CH₂), 2.21 (t, 2H, J=7.5 Hz, CH₂), 2.12-2.05 (m, 2H, CH₂), 1.99-1.92 (m, 8H, CH₂), 1.84-1.57 (m, 24H, CH₂, CH₃), 1.52-1.16 (m, 38H, CH, CH₂), 1.00-0.87 (m, 7H, CH₂, CH₃) ppm; ¹³C-NMR (150 MHz, MeOD) δ=176.6 (C_{q}), 176.4 (C_{q}), 175.9 (C_{q}), 174.8 (C_{q}), 174.0 (C_{q}), 173.6 (C_{q}), 169.9 (C_{q}), 159.8 (C_{q}), 148.8 (CH_{aromatisch}), 144.9 (C_{q}), 143.6 (C_{q}), 142.4 (C_{q}), 128.3 (CH_{aromatisch}), 124.5 (CH_{aromatisch}), 121.4 (CH_{aromatisch}), 111.9 (CH_{aromatisch}), 106.7 (CH_{aromatisch}), 67.7 (CH₂), 55.2 (CH), 54.2 (CH), 52.8 (O—CH₃), 51.7 (CH, CH₂), 50.4 (C_{q}), 45.4 (CH₂), 45.3 (CH₂), 41.0 (CH₂), 40.5 (CH₂), 40.1 (CH₂), 37.3 (CH₂), 37.1 (CH₂), 36.8 (CH₂), 35.4 (CH), 34.8 (CH₂), 33.7 (CH₂), 33.1 (CH₂), 32.5 (CH₂), 31.7 (CH₂), 31.2 (CH₂), 30.8 (CH₂), 30.7 (CH₂), 30.5 (CH₂), 30.4 (CH₂), 30.2 (CH₂), 30.1 (CH₂), 28.3 (CH₃), 28.1 (CH₂), 27.7 (CH₂), 27.4 (CH₂), 27.3 (CH₂), 26.9 (CH₂), 24.5 (CH₂), 23.7 (CH₂), 23.5 (CH₂), 14.5 (CH₃) ppm.

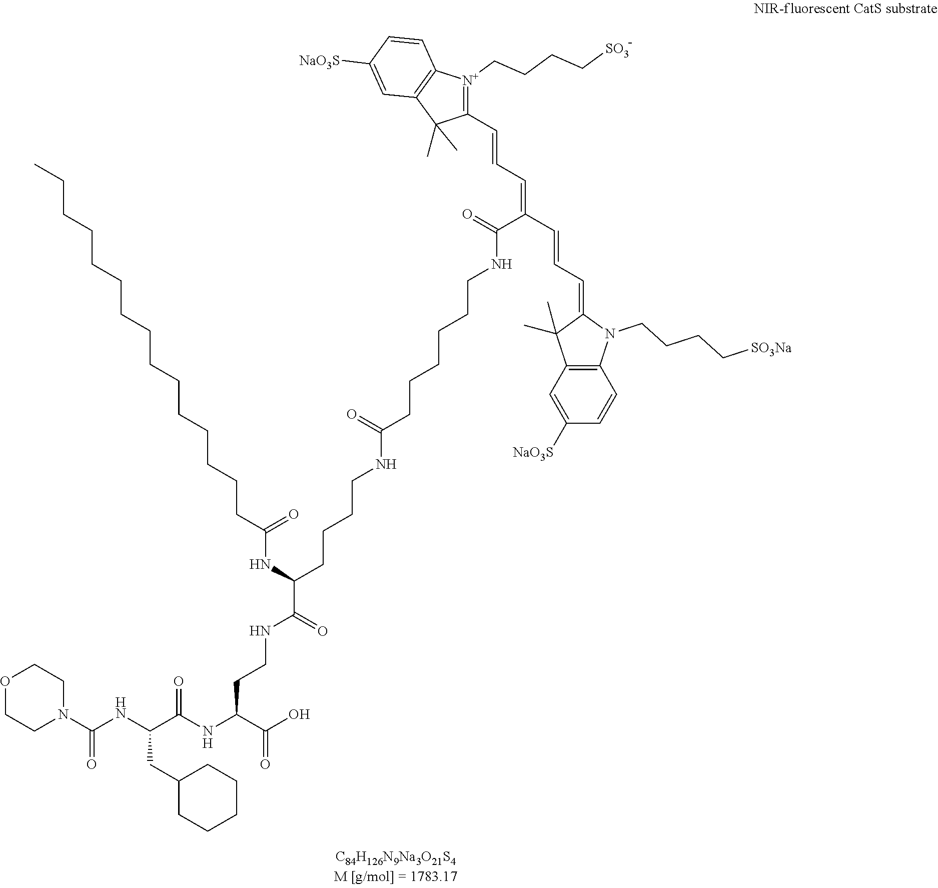

Methylester ber4 was solved in THF/H₂O (3:1, 800 μL, 0.003 M) and mixed with a 0.6 M solution of LiOH*H₂O in H₂O (24 μL, 0.014 mmol, 8.0 equiv.) and stirred for 2 h at room temperature and neutralized by the addition of acetic acid (814 μL, 0.014 mmol, 8.0 equiv.). The reaction solution was diluted with H₂O and lyophilised. Column chromatographic purification (C18-WP, H₂O(TFA)/ACN(TFA)=5-95%) afforded NIR-fluorescent CatS substrate (1.7 mg, 0.002 mmol, 86%) a's a green solid.

LCMS (Thermofisher PFP): t_{R}=8.11, m/z=859 [M−3Na+5H]²⁺; ESI−HRMS: m/z calc. für C₈₃H₁₂₉N₉O₂₁S₄Na [M−2Na+3H]⁺: 1738.8084, gefunden: 1738.8113; ¹H-NMR (500 MHz, MeOD) δ=7.93-7.82 (m, 6H, CH_{aromatisch}), 7.40 (d, 2H, J=8.3 Hz, CH_{aromatisch}), 6.53 (dd, 4H, J=33.0, 13.4 Hz, CH_{aromatisch}), 4.43 (dd, 1H, J=10.3, 4.3 Hz, CH), 4.31 (dd, 1H, J=9.1, 6.2 Hz, CH), 4.23-4.11 (m, 5H, CH, CH₂), 3.67-3.60 (m, 4H, CH₂), 3.46-3.39 (m, 4H, CH₂), 3.19-3.14 (m, 2H, CH₂), 2.90 (t, 4H, J=6.6 Hz, CH₂), 2.25-2.08 (m, 6H, CH₂), 2.01-1.92 (m, 8H, CH₂), 1.84-1.57 (m, 30H, CH₂, CH₃), 1.53-1.17 (m, 37H, CH, CH₂), 0.99-0.87 (m, 7H, CH₂, CH₃) ppm; ¹³C-NMR (150 MHz, MeOD) δ=176.5 (C_{q}), 176.4 (C_{q}), 176.0 (C_{q}), 174.7 (C_{q}), 174.0 (C_{q}), 169.9 (C_{q}), 159.8 (C_{q}), 148.8 (CH_{aromatisch}), 144.9 (C_{q}), 143.5 (C_{q}), 142.4 (C_{q}), 130.8 (C_{q}), 130.2 (C_{q}), 128.3 (CH_{aromatisch}), 127.9 (CH_{aromatisch}), 124.4 (CH_{aromatisch}), 111.9 (CH_{aromatisch}), 106.7 (CH_{aromatisch}), 67.7 (CH₂), 55.2 (CH), 54.2 (CH), 51.7 (CH₂), 51.6 (CH), 50.5 (C_{q}), 45.4 (CH₂), 45.3 (CH₂), 41.0 (CH₂), 40.4 (CH₂), 40.1 (CH₂), 37.3 (CH₂), 37.1 (CH₂), 36.8 (CH₂), 35.3 (CH), 34.9 (CH₂), 33.6 (CH₂), 33.1 (CH₂), 32.6 (CH₂), 31.7 (CH₂), 30.8 (CH₂), 30.7 (CH₂), 30.5 (CH₂), 30.2 (CH₂), 30.1 (CH₂), 28.3 (CH₃), 28.1 (CH₂), 27.7 (CH₂), 27.4 (CH₂), 27.3 (CH₂), 27.0 (CH₂), 26.9 (CH₂), 24.5 (CH₂), 23.7 (CH₂), 23.5 (CH₂), 14.5 (CH₃) ppm.

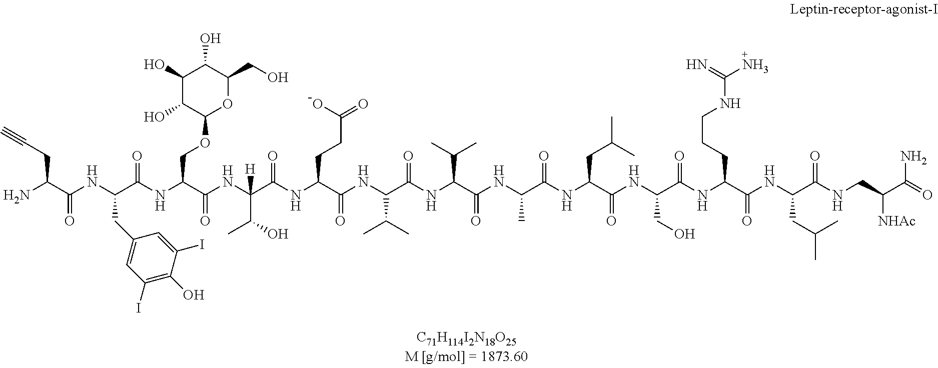

Leptin-receptor-agonist-I was synthesized according to literature (22), with the difference that an additional propargyl glycine was added at the N-terminus. Synthesis was performed using Fmoc solid phase synthesis.

| | | | t_{R} | |
|---|---|---|---|---|
| Sequence | modified residues | yield | [min] | m/z |
| XYSTEVVALSRLQ | X = propargylglycine | 16% | 8.28 | 937.87 (2+) |
| 06-009 | Y = Tyr(3,5-I₂) | | | calc: |
| | S = O- -Glucopyranosyl-serine | | | 937.32 (2+) |
| | Q = 2-acetamido-3-amino propionic | | | |
| | acid | | | |

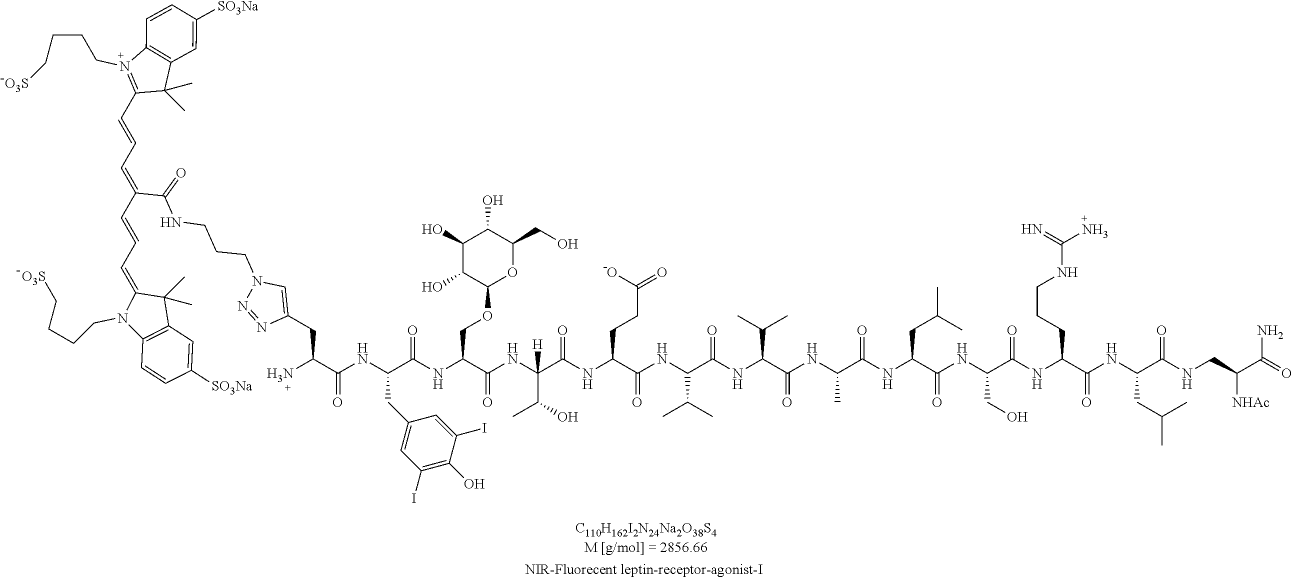

Leptin-receptor-agonist-I (4.4 μmol) and dyeazid3 (4.6 mg, 4.6 μmol, 1.1 eq) were dissolved in water (1.0 mL). Aminoguanidin-HCl (50 mM in water, 527 μL, 26.3 μmol, 6.0 eq), THPTA (50 mM in water, 263 μL, 13.2 μmol, 3.0 eq) was added, then CuSO₄ (20 mM in water, 658 μL, 13.2 μmol, 3.0 eq). Sodium ascorbate (500 mM in water, freshly dissolved, 53 μL, 26.3 μmol, 6.0 eq) was added and it was stirred until LCMS indicated completeness of reaction (1-2 h). The reaction mixture was loaded directly on a C₁₈-Flash-Cartridge (4 g) and chromatographed (Eluent: water/acetonitril, 0.05% TFA). NIR-fluorescent leptin-receptor-agonist-I was obtained a's dark green solid.

| | | | t_{R} | |
|---|---|---|---|---|
| Sequence | modified residues | yield | [min] | m/z |
| XYSTEVVALSRLQ | X = propargylglycine, clicked to | 57% | 9.3 | 1406.4362 (2+) |
| 06-011 | dyeazid3 | | | calc: |
| | Y = Tyr(3,5-I2) | | | 1406.4384 (2+) |
| | S = O- -Glucopyranosyl-serine | | | |
| | Q = 2-acetamido-3-amino propionic | | | |
| | acid | | | |

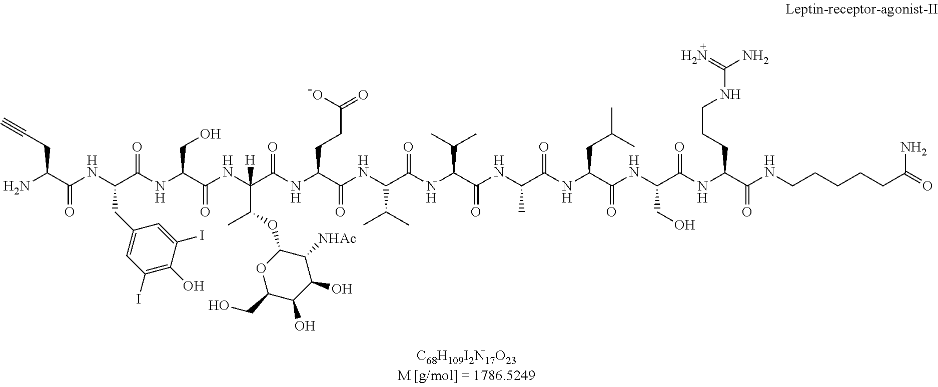

Leptin-receptor-agonist-II was obtained in a similar manner. Therefore N-terminally propargyl glycine elongated derivative was synthesized according to literature (23).

| | | | t_{R} | |
|---|---|---|---|---|
| Sequence | modified residues | yield | [min] | m/z |
| XYSTEVVALSRLQ | X = propargylglycine | 38% | 6.38 | 893.82 (2+), |
| 06-077 | Y = Tyr(3,5-I₂) | | | 596.58 (3+) |
| | T = O- -(2-acetamido-2-deoxy- | | | calc: |
| | Galactopyranosyl-threonine | | | 893.81 (2+), |
| | Q = 6-aminocaproic acid | | | 596.21 (3+) |

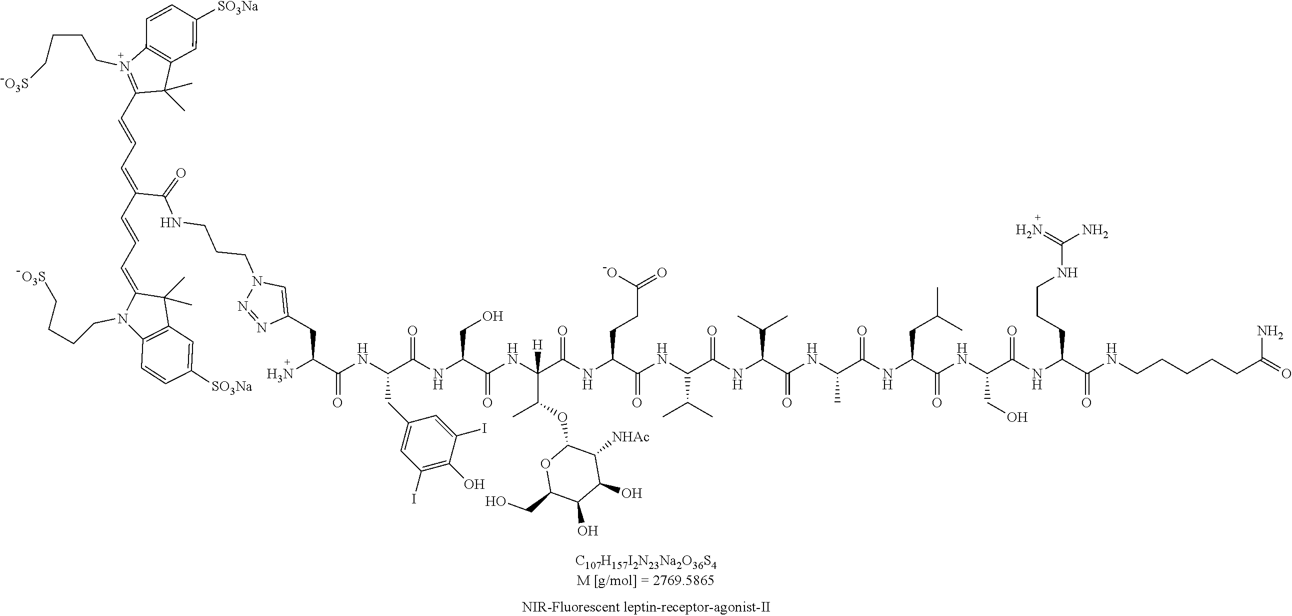

Conjugation of dyeazid3 was performed in a similar manner a's described above to yield NIR-fluorescent leptin-receptor-agonist-II.

| | | | t_{R} | |
|---|---|---|---|---|
| Sequence | modified residues | yield | [min] | m/z |
| XYSTEVVALSRLQ | X = propargylglycine, clicked to | 60% | 6.44 | 908.77 (3+) |
| 06-080 | dyeazid3 | | | calc: |
| | Y = Tyr(3,5-I2) | | | 908.95 (3+) |
| | T = O- -(2-acetamido-2-deoxy- | | | |
| | Galactopyranosyl-threonine | | | |
| | Q = 6-aminocaproic acid | | | |

### REFERENCES

1. ACS Nano. 2016 May 24; 10(5): 5015-5026, FIG. 10
2. Clin Cancer Res; 18(20) Oct. 15, 2012
3. S̆tacková L. et al.; J. Am. Chem. Soc. 2019, 141, 17, 7155-7162
4. https://www.licor.com/bio/targeted-therapeutics-development/clinical-applications
5. US0221512A1
6. Roger R. et al; Org. Lett. 2015, 17, 302-305
7. Li et al.; Angew. Chem. Int. Ed. 2020, 59, 12154-12161
8. Luciano et al.; ACS Chem. Biol. 2019, 14, 934-940
9. WO 2011/116142A1
10. WO 2020/020905 A1
11. WO 00/71162 A2
12. US 2008/0233050 A1
13. Vargas, S. H. et al.; Clin Cancer Res; 25(14) Jul. 15, 201
14. U.S. Pat. No. 10,441,607 B1
15. Dommerholt, J., Rutjes, F. P. J. T. & van Delft, F. L. Strain-Promoted 1,3-Dipolar Cycloaddition of Cycloalkynes and Organic Azides. Top. Curr. Chem. 374, 1-20 (2016).
16. Liu, Z., Derosa, J. & Engle, K. M. Palladium(II)-Catalyzed Regioselective syn-Hydroarylation of Disubstituted Alkynes Using a Removable Directing Group. J. Am. Chem. Soc. 138, 13076-13081 (2016).
17. Cao, H. et al. A red Cy3-based biarsenical fluorescent probe targeted to a complementary binding peptide. J. Am. Chem. Soc. 129, 8672-8673 (2007).
18. Schottelius, M., Wester, H. J., Reubi, J. C., Senekowitsch-Schmidtke, R. & Schwaiger, M. Improvement of pharmacokinetics of radioiodinated Tyr3-octreotide by conjugation with carbohydrates. Bioconjug. Chem. 13, 1021-1030 (2002).
19. Jadhav, S. V., Bandyopadhyay, A., Benke, S. N., Mali, S. M. & Gopi, H. N. A facile synthesis and crystallographic analysis of N-protected β-amino alcohols and short peptaibols. Org. Biomol. Chem. 9, 4182-4187 (2011).
20. Weineisen et al. EJNMMI Research 2014, 4:63
21. Angew. Chem. Int. Ed. 2014, 53, 7669-7673.
22. Biochimica et Biophysica Acta 1783 (2008) 1745-1754.
23. Diabetes, Obesity and Metabolism 12: 393-402, 2010.

1. A compound according to formula (I), 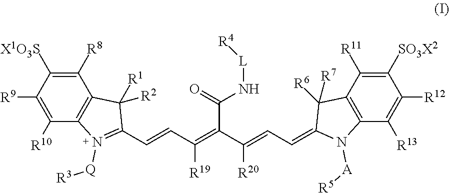
wherein
R¹, R², R⁶, R⁷, R¹⁴, R¹⁵, R¹⁶, R¹⁷, and R¹⁸ are independently selected from the group consisting of H and (C₁-C₆)alkyl;
R³ is —SO₃X³⁻ or COOH;
R⁴ is selected from the group consisting of -ethinyl, -azide, 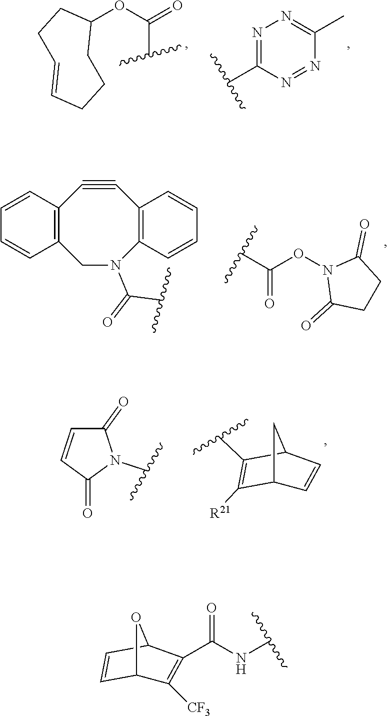
—COOH, NH₂, Cl, Br, I, —OSO₂CF₃, —COSO₂CH₃, —OH, vinyl, —SH;
R⁵ is —SO₃X⁴;
R⁸, R⁹, R¹⁰, R¹¹, R¹² and R¹³ are independently selected from the group consisting of H, CN, —COO(C₁-C₆)alkyl, —CONH(C₁-C₆)alkyl, Cl, Br, I, F, —CF₃, —O(C₁-C₆), —NR¹⁴R¹⁵, —NHC(O)(C₁-C₆)alkyl, —NHC(O)NH(C₁-C₆)alkyl), —SO₃H, —SO₂NH₂, -(C₁-C₆)alkyl;
R¹⁹, R²⁰ are independently selected from the group consisting of H, -(C₁-C₆)alkyl, —O(C₁-C₆)alkyl, Cl, F, Br, CN; C(O)NH₂;
R²¹ is H, -(C₁-C₁₀)alkyl;
L is selected from the group: —(CH₂)ₙ₁—, -(C₁-C₆)cycloalkylene-, -(C₁-C₆)heterocycloalkylene-, —O—, —NR¹⁶—, —N—, —NHC(O)—, —C(O)NR¹⁷—, —CH(OH)—, —CH(OR¹⁸)—, and combinations thereof;
n¹ is an integer between 1 to 30;
X¹, X², X³, and X⁴ are independently selected from a cation, a proton, or absent;
Q is —(CH₂)ₙ₂—,
wherein optionally one or more of each H in —(CH₂)ₙ₂— may be replaced by -(C₁-C₁₀)alkyl;
n² is an integer between 1 to 10; preferably 1 to 1;
A is —(CH₂)ₙ₃—,
wherein optionally one or more of each H in —(CH₂)ₙ₃— may be replaced by -(C₁-C₁₀)alkyl;
n³ is an integer between 1 to 10.

2. The compound of claim 1, wherein
R¹, R², R⁶, R⁷ are each methyl;
R³ is —SO₃X³;
R⁴ is selected from the group consisting of -ethinyl, -azide, 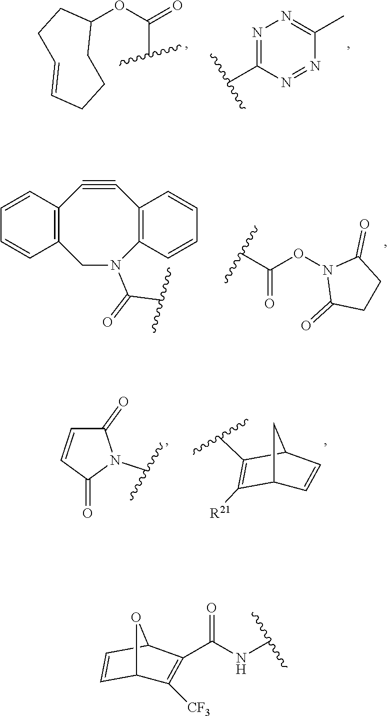
—COOH, and NH₂;
R⁵ is —SO₃X⁴;
R⁸, R⁹, R¹⁰, R¹¹, R¹², and R¹³ are H;
R¹⁶, R¹⁷, and R¹⁸ are independently selected from the group consisting of H and (C₁-C₆)alkyl;
R¹⁹ and R²⁰ are H;
R²¹ is H, -(C₁-C₁₀)alkyl;
L is selected from the group: —(CH₂)ₙ₁—, —O—, —NR¹⁶—, —NHC(O)—, —C(O)NR¹⁷—, —CH(OH)—, —CH(OR¹⁸)—, and combinations thereof;
n¹ is an integer between 1 to 30;
X¹, X², X³, and X⁴ are independently selected from a cation, a proton or absent;
Q is —(CH₂)ₙ₂—,
wherein optionally one or more of each H in —(CH₂)ₙ₂— may be replaced by -(C₁-C₁₀)alkyl;
n² is an integer between 1 to 10;
A is —(CH₂)ₙ₃—,
wherein optionally one or more of each H in —(CH₂)ₙ₃— may be replaced by -(C₁-C₁₀)alkyl;
n³ is an integer between 1 to 10.

3. The compound of claim 1, wherein the compound is selected from

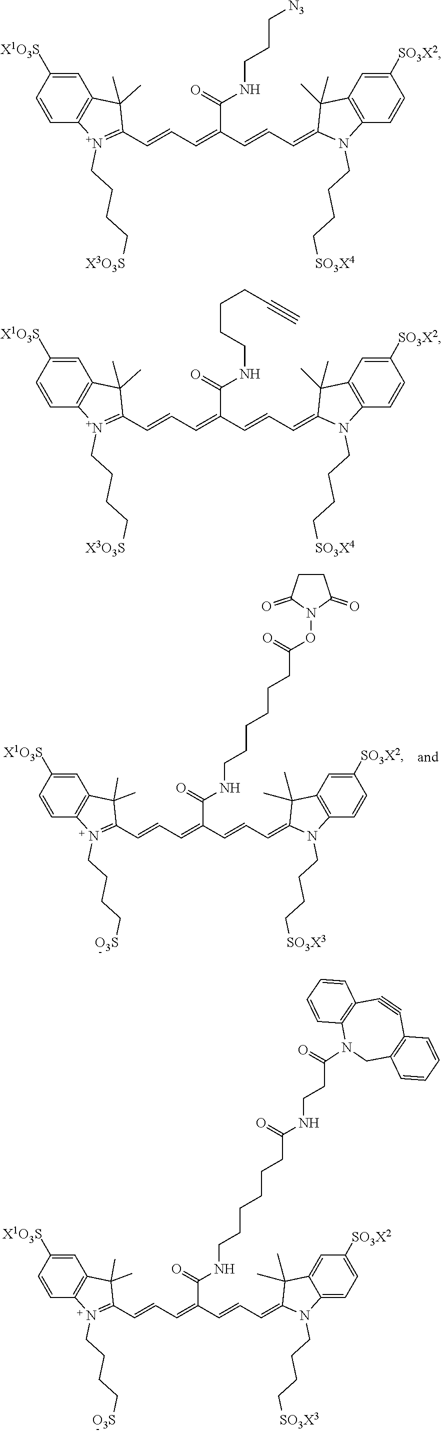

4. A compound according to formula (II) 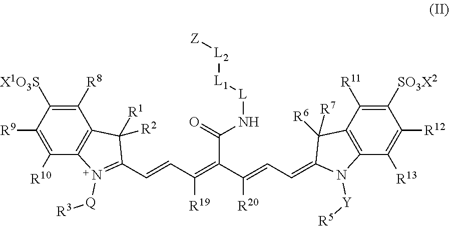
wherein
R¹, R², R⁶, R⁷, R¹⁴, R¹⁵, R¹⁶, R¹⁷, and R¹⁸ are independently selected from the group consisting of H and (C₁-C₆)alkyl;
R³ is —SO₃X³, or COOH;
Z is a protein, mono- or polyvalent saccharide, peptide, small molecule, antibody, oligonucleotide, nanobody, or polymer;
R⁵ is —SO₃X⁴;
R⁸, R⁹, R¹⁰, R¹¹, R¹² and R¹³ are independently selected from the group consisting of H, CN, —COO(C₁-C₆)alkyl, —CONH(C₁-C₆)alkyl, Cl, Br, I, F, —CF₃, —O(C₁-C₆), —NR¹⁴R¹⁵, —NHC(O)(C₁-C₆)alkyl, —NHC(O)NH(C₁-C₆)alkyl), —SO₃H, —SO₂NH₂, -(C₁-C₆)alkyl;
R¹⁹, R²⁰ are independently selected from the group consisting of H, -(C₁-C₆)alkyl, —O(C₁-C₆)alkyl, Cl, F, Br, CN; C(O)NH₂;
R²¹ is H, -(C₁-C₁₀)alkyl;
X¹, X², X³, and X⁴ are independently selected from a cation, a proton or absent;
Q is —(CH₂)ₙ₂—,
wherein optionally one or more of each H in —(CH₂)ₙ₂— may be replaced by -(C₁-C₁₀)alkyl;
n² is an integer between 1 to 10; preferably 1 to 1;
Y is —(CH₂)ₙ₃—,
wherein optionally one or more of each H in —(CH₂)ₙ₃— may be replaced by -(C₁-C₁₀)alkyl;
n³ is an integer between 1 to 10;
L is selected from the group: —(CH₂)ₙ₁—, -(C₁-C₆)cycloalkylene-, -(C₁-C₆)heterocycloalkylene-, —O—, —NR¹⁶−, —NH—, —NHC(O)—, —C(O)NR¹⁷—, —CH(OH)—, —CH(OR¹⁸)—, and combinations thereof;
n¹ is an integer between 1 to 30;
L₁ is selected from the group consisting of 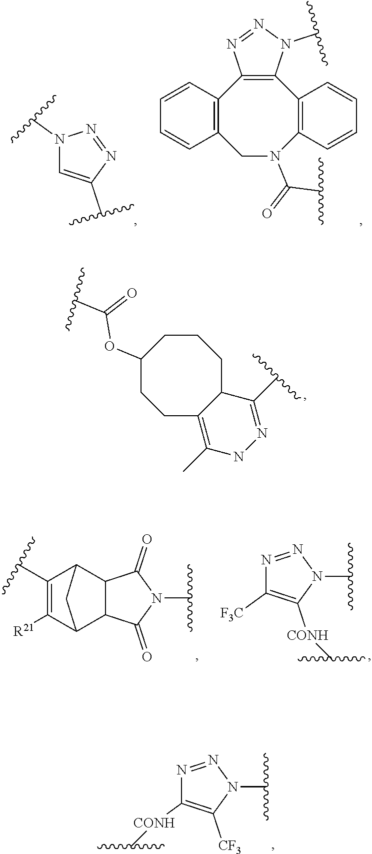
—CO—;
L₂ is a selected from the group: —(CH₂)ₙ₁—, -(C₁-C₆)cycloalkylene-, -(C₁-C₆)heterocycloalkylene-, —O—, —NR¹⁶—, —NH—, —NHC(O)—, —C(O)NR¹⁷—, —CH(OH)—, —CH(OR¹⁸)—, and combinations thereof.

5. The compound of claim 4, wherein
R¹, R², R⁶, R⁷ are each methyl;
R³ is —SO₃X³;
R⁵ is —SO₃X⁴;
R⁸, R⁹, R¹⁰, R¹¹, R¹² and R¹³ are H;
R¹⁶, R¹⁷, and R¹⁸ are independently selected from the group consisting of H and (C₁-C₆)alkyl;
R¹⁹ and R²⁰ are H;
X¹, X², X³, and X⁴ are independently selected from a cation, a proton or absent;
Q is —(CH₂)ₙ₂—,
wherein optionally one or more of each H in —(CH₂)ₙ₂— may be replaced by -(C₁-C₁₀)alkyl;
n² is an integer between 1 to 10;
Y is —(CH₂)ₙ₃—,
wherein optionally one or more of each H in —(CH₂)ₙ₃— may be replaced by -(C₁-C₁₀)alkyl;
n³ is an integer between 1 to 10;
L is selected from the group: —(CH₂)ₙ₁—, —O—, —NR¹⁶—, —NHC(O)—, —C(O)NR¹⁷—, —CH(OH)—, —CH(OR¹⁸)—, and combinations thereof;
n¹ is an integer between 1 to 30;
n⁴ is an integer between 1 to 10;
L₁ is selected from the group consisting of 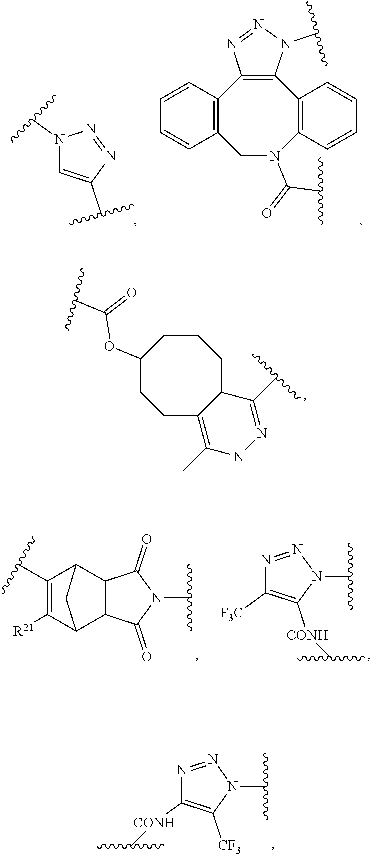
—CO—,
R²¹ is H, -(C₁-C₁₀)alkyl.

6. The compound of claim 4, wherein Z is selected from the group consisting of Octreotide, Octreotate, TOC, TATE, NP41, Leptin, Insulin, Glucagon, GLP-1, methanobactin OB3b, PSMA (Prostate-Specific Membrane Antigen), PSMA binding ligands like KUE, EGFR targeting antibodies, cetuximab, panitutumab, HER2 targeting antibodies, trastuzumab, CEA targeting antibodies, VEGFR targeting antibodies, bevatuzumab, and αvβ6-integrin binders.

7. The compound of claim 4, wherein L₁ is selected from the group consisting of 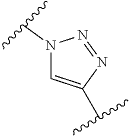 [SMILES: *c1cn(*)nn1]
and —CO—, and
n⁴ is an integer between 1 to 10.

8. The compound of claim 1, wherein L is selected from the group consisting of —CH₂(CH₂OCH₂)ₙ₁CH₂—, —(CH₂)ₙ₂—, —CH₂(CH₂NHCH₂)ₙ₁CH₂—, —CH₂(CH₂NR¹⁶CH₂)ₙ₁CH₂—, —CH₂(CH₂OCH₂)ₙ₁CH₂C(O)(CH₂)ₙ₅—, —CH₂(CH₂NHCH₂)ₙ₁CH₂C(O)(CH₂)ₙ₅—, —CH₂(CH₂NR¹⁶CH₂)ₙ₁CH₂C(O)(CH₂)ₙ₅—;
n¹ is an integer between 1 to 30;
n² is an integer between 1 to 10;
n⁵ is an integer between 1 to 10.

9. The compound of claim 4, wherein the compound is selected from the group consisting of:

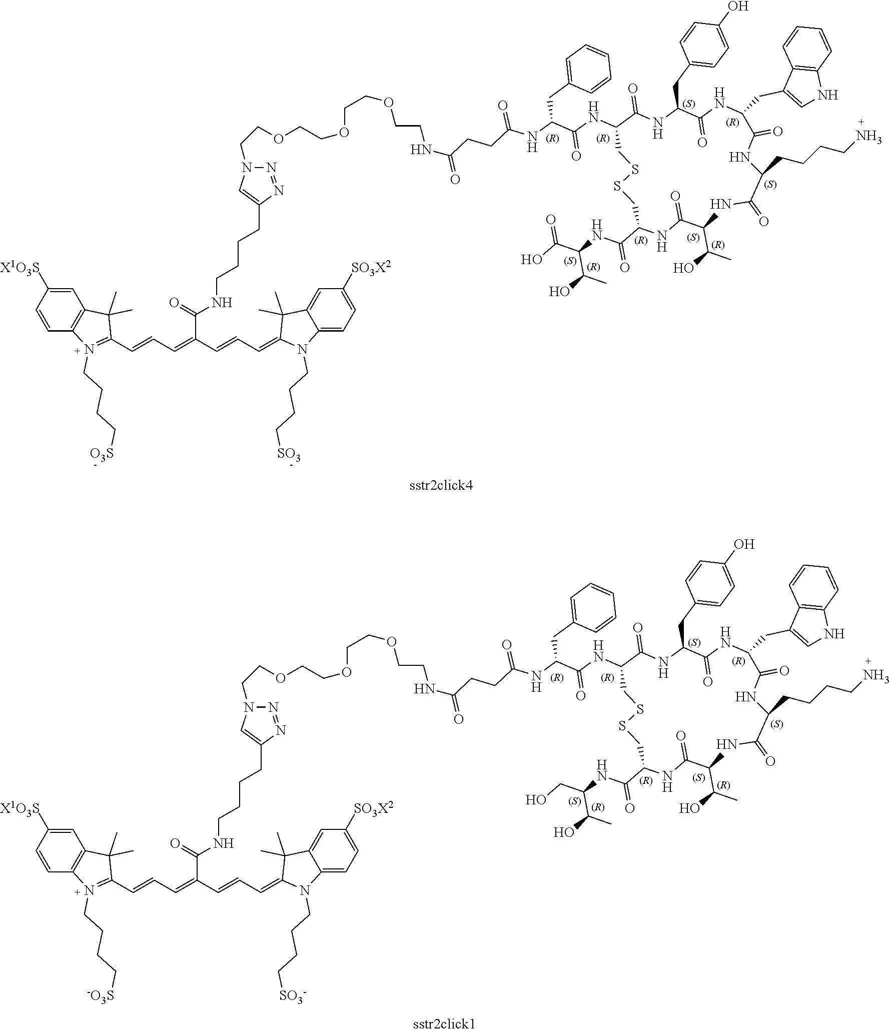

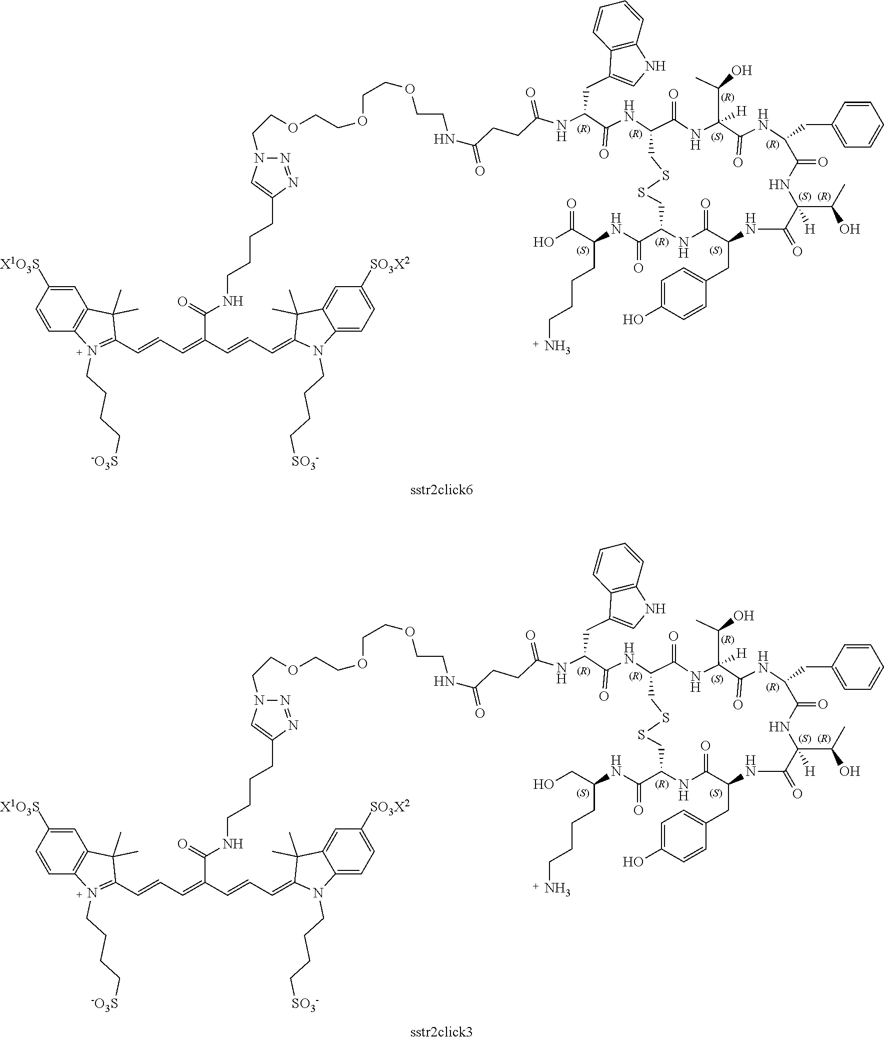

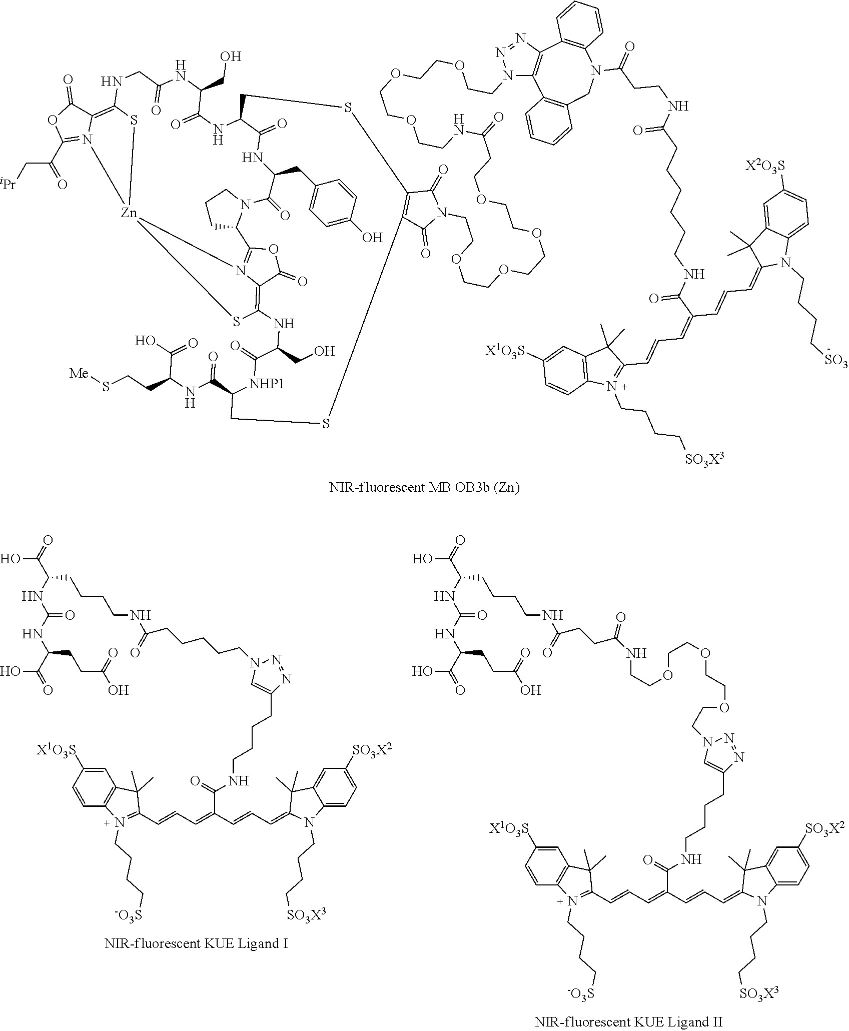

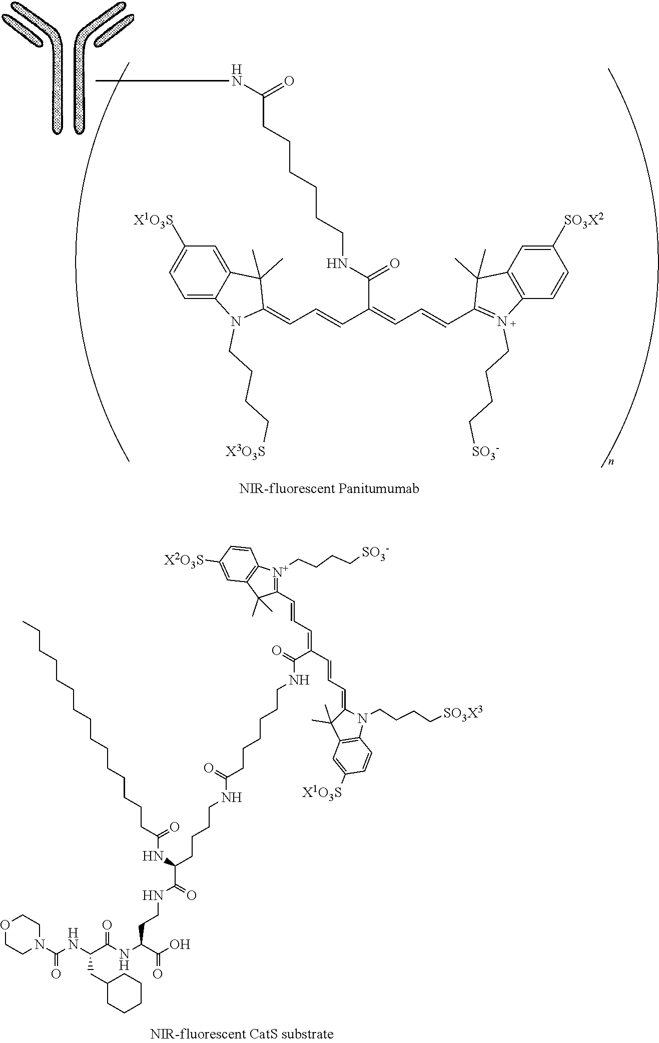

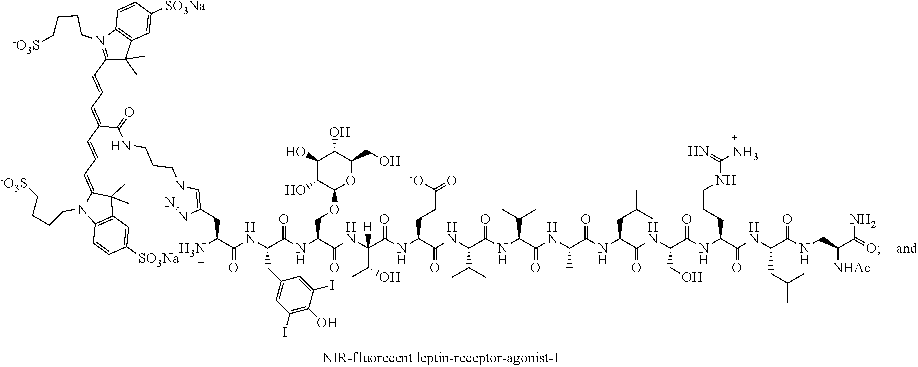

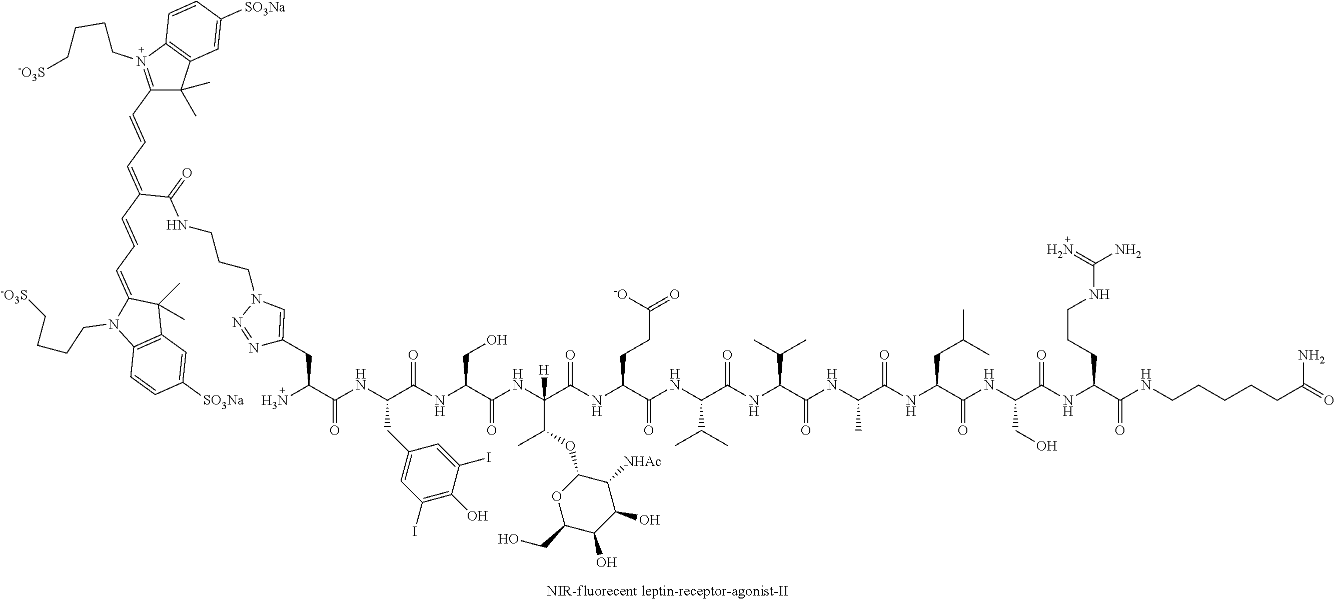

10. The compound of claim 1, wherein
i) the peak emission in PBS is between 400 nm to 1200 nm, and/or
ii) the absorption crosssection in PBS is greater than 5,000 1/(M*cm), and/or
iii) the peak absorption in PBS is from 400 nm to 1200 nm.

11. The compound of claim 1, wherein the compound is a fluorescent dye.

12. The compound of claim 1, wherein the compound is a contrast agent.

13-15. (canceled)

16. The compound of claim 1, wherein at least one of R¹, R², R⁶, R⁷, R¹⁴, R¹⁵, R¹⁶, R¹⁷, and R¹⁸ is methyl.

17. A method of diagnosing an indication in a subject in need thereof, comprising the step of administering a compound according to formula (II) 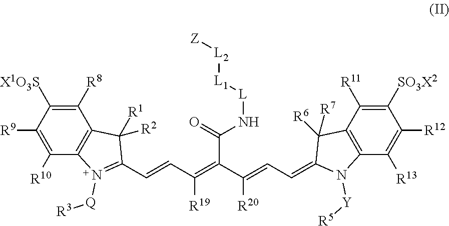
wherein
R¹, R², R⁶, R⁷, R¹⁴, R¹⁵, R¹⁶, R¹⁷, and R⁸ are independently selected from the group consisting of H and (C₁-C₆)alkyl;
R³ is —SO₃X³, or COOH;
Z is a protein, mono- or polyvalent saccharide, peptide, small molecule, antibody, oligonucleotide, nanobody, or polymer;
R⁵ is —SO₃X⁴;
R⁸, R⁹, R¹⁰, R¹¹, R¹² and R¹³ are independently selected from the group consisting of H, CN, —COO(C₁-C₆)alkyl, —CONH(C₁-C₆)alkyl, Cl, Br, I, F, —CF₃, —O(C₁-C₆), —NR¹⁴R¹⁵, —NHC(O)(C₁-C₆)alkyl, —NHC(O)NH(C₁-C₆)alkyl), —SO₃H, —SO₂NH₂, -(C₁-C₆)alkyl;
R¹⁹, R²⁰ are independently selected from the group consisting of H, -(C₁-C₆)alkyl, —O(C₁-C₆)alkyl, Cl, F, Br, CN; C(O)NH₂;
R²¹ is H, -(C₁-C₁₀)alkyl;
X¹, X², X³, and X⁴ are independently selected from a cation, a proton or absent;
Q is —(CH₂)ₙ₂—,
wherein optionally one or more of each H in —(CH₂)ₙ₂— may be replaced by -(C₁-C₁₀)alkyl;
n² is an integer between 1 to 10;
Y is —(CH₂)ₙ₃—,
wherein optionally one or more of each H in —(CH₂)ₙ₃— may be replaced by -(C₁-C₁₀)alkyl;
n³ is an integer between 1 to 10;
L is selected from the group: —(CH₂)ₙ₁—, -(C₁-C₆)cycloalkylene-, -(C₁-C₆)heterocycloalkylene-, —O—, —NR¹⁶—, —NH—, —NHC(O)—, —C(O)NR¹⁷—, —CH(OH)—, —CH(OR¹⁸)—, and combinations thereof;
n is an integer between 1 to 30;
L₁ is selected from the group consisting of 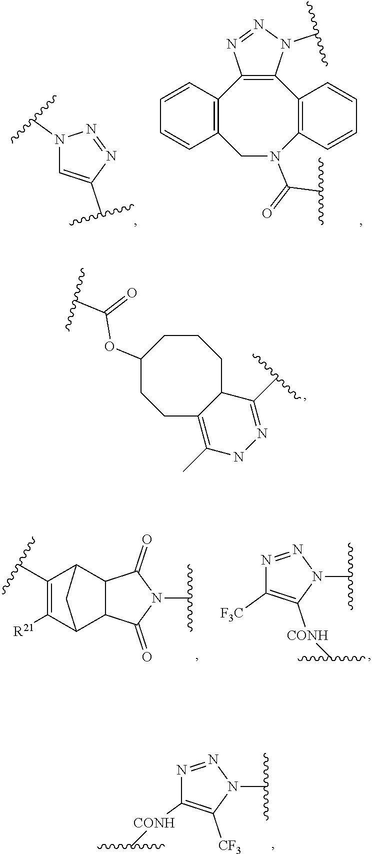
—CO—;
L₂ is selected from the group: —(CH₂)ₙ₁—, -(C₁-C₆)cycloalkylene-, -(C₁-C₆)heterocycloalkylene-, —O—, —NR¹⁶—, —NH—, —NHC(O)—, —C(O)NR¹⁷—, —CH(OH)—, —CH(OR¹⁸)—, and combinations thereof; and
irradiating the compound with light having a wavelength between 400 nm and 1200 nm.

18. The method of claim 17, wherein the indication is selected from the group consisting of an inflammatory disease, an infectious disease, and a cancer.

19. The method of claim 18, wherein the inflammatory disease is selected from the group consisting of otitis media, allergic rhinitis and chronic rhinosinusitis, chronic inflammation of tonsils, adenoids, and laryngitis.

20. The method of claim 18, wherein the infectious disease is selected from the group consisting of implant infection, hip implant infection, dental implant infection, a secondary infection of the inner ear, accompanying cholesteatoma, an infection caused by gram positive bacteria, an infection caused by gram negative bacteria, an infection responding to antibiotic treatment, resistant infection, multi-drug resistant infection, and an infection caused by a bacteria selected from Enterococcus faecium, Staphylococcus aureus, Klebsiella pneumoniae, Acinetobacter baumannii, Pseudomonas aeruginosa, and Enterobacter spp..

21. The method of claim 18, wherein the cancer is a primary tumor or a metastatic lesion selected from the group meningioma, glioblastoma, NSCLC, pancreatic cancer, neuroendocrine tumors, lung cancer, breast cancer, and meningiomas.

22. The method of claim 17, wherein the method diagnoses an indication for intraoperative surgery.

23. The method of claim 22, wherein the intraoperative surgery comprises visualizing a primary tumor or a metastatic lesion in situ.
